(19) United States
(12) Patent Application Publication
(10) Pub. No.: US 2025/0312336 A1
(43) Pub. Date: Oct. 9, 2025

(54) Compositions And Methods For Treating Cancer With Calcium Channel Inhibitors

(71) Applicant: Institute for Cancer Research d/b/a The Research Institute of Fox Chase Cancer Center, Philadelphia, PA (US)

(72) Inventors: Igor Astsaturov, Philadelphia, PA (US); Edna Cukierman, Philadelphia, PA (US); Jonathan Soboloff, Philadelphia, PA (US)

(21) Appl. No.: 19/174,410

(22) Filed: Apr. 9, 2025

Related U.S. Application Data
(60) Provisional application No. 63/631,921, filed on Apr. 9, 2024.

(51) Int. Cl. A61K 31/497; A61K 31/167; A61K 31/415; A61K 31/433; A61K 31/44; A61K 45/06

(52) U.S. Cl. CPC A61K 31/497; A61K 31/167; A61K 31/415; A61K 31/433; A61K 31/44; A61K 45/06

(57) ABSTRACT

Disclosed are methods of treating cancer in a subject comprising administering to the subject a calcium channel inhibitor. Disclosed are methods of decreasing ANO6 activity in cancer-associated fibroblasts (CAFs) comprising contacting a CAF with a calcium channel inhibitor. Disclosed are pharmaceutical compositions comprising an Orai channel inhibitor; an anti-cancer agent; and a pharmaceutically acceptable carrier.

### CROSS-REFERENCE TO RELATED APPLICATIONS

This application claims the benefit of U.S. Provisional Patent Application No. 63/631,921, filed Apr. 9, 2024, which is incorporated by reference herein in its entirety.

### STATEMENT REGARDING FEDERALLY SPONSORED RESEARCH

This invention was made with government support under CA006927 awarded by National Institutes of Health. The government has certain rights in the invention.

### BACKGROUND

Cancer cells are characterized by increased requirement for lipids to sustain their growth and to promote membrane-based receptor signaling. As de novo lipid synthesis consumes energy and oxygen, most aggressive human cancers resort to metabolic parasitism, i.e., uptake of cholesterol and other lipids from exogenous sources. While tumors normally acquire lipids and other nutrients from the bloodstream, the PDAC tumor mass is remarkable in its lack of functional intratumoral blood vessels, consisting predominantly of dense connective tissue populated by cancer associated fibroblasts. How the pancreatic cancer cells obtain their essential nutrients has thus been mysterious but of high interest, as determining how PDAC cells acquire exogenous lipids could facilitate a potential therapeutic strategy based on blockade of this process. In the past, some have proposed a direct uptake of lipids from the interstitial fluid, given high levels of larger lipid particles and soluble nutrients can be found in this compartment in PDACs. Lack of tangible targets and incomplete understanding of the delivery mechanism has been the major obstacle for efficacious restriction of lipids in blood or interstitial fluid experimentally or clinically. As the result, a continuous stream of exogenous lipids including cholesterol, from blood to the interstitial fluid, overrides the dietary lipid restriction and abrogates the activity of the de novo cholesterol biosynthesis inhibitors such as statins.

### BRIEF SUMMARY

Disclosed are methods of treating cancer, comprising administering to a subject in need thereof a calcium channel inhibitor.

Disclosed are methods of decreasing ANO6 activity in a cancer-associated fibroblast (CAF) comprising contacting a CAF with a calcium channel inhibitor.

Disclosed are compositions comprising an Orai channel inhibitor and an anti-cancer agent.

Disclosed are compositions comprising: an Orai channel inhibitor; an Ano6 inhibitor; and a pharmaceutically acceptable carrier.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1H show delivery of low-density lipoprotein particles to pancreatic cancer cells is mediated by cancer-associated fibroblasts. (FIG. 1A) Cryosections of KPC autochthonous tumors 15 minutes after intravenous injection of 100 μl of LDL particles labeled with BODIPY-conjugated cholesterol. BV, blood vessel; arrowheads, LDL (green) in the interstitial spaces of the tumor. (B, C) Cryosection of KPC tumors 90 minutes (FIG. 1B) and 20 hours (FIG. 1C) after intravenous injection of LDL particles labeled with BODIPY-conjugated cholesterol. (FIG. 1D) Integrated intensity of BODIPY-cholesterol at indicated times in epithelial (cytokeratin positive) and fibroblastic (podoplanin positive) compartments. Right Y-axis, concentration of labeled LDL in serum represented as mean±SEM. Boxes represent upper and lower quartile limits, horizontal line-median, and individual measurements are shown as circles. **, p<0.01 comparing podoplanin and cytokeratin-positive areas (unpaired two-tailed t-test). (FIG. 1E) Percent co-localization of BODIPY-cholesterol in DsRed-tagged carcinoma or stromal cells over time. Graph represents pooled images of pancreatic tumors from 2-3 anesthetized animals imaged at 1-2-hour intervals by multiphoton microscopy. (FIG. 1F) Intravital microscopy images of orthotopic DsRed-tagged (red) pancreatic tumors at 15 minutes, 90 minutes, and 17 hours after intravenous injection of donor LDL labeled with BODIPY-conjugated cholesterol (green). AT, acinar tissue; BV, blood vessel; T, tumor. (FIG. 1G) Flow cytometry enumeration of LDL label in subsets of cells from disintegrated tumors 90 minutes post intravenous administration of fluorescent LDL. Data are represented as mean±SEM. **, p<0.01, ***, p<0.001 as compared with CAFs (unpaired two-tailed t-test). (FIG. 1H) Enhanced intravital images of endosomal aggregates containing BODIPY-cholesterol and cancer cells in pancreatic tumors. Red, KPCN349 murine pancreatic carcinoma cells expressing DsRed; green, BODIPY-conjugated cholesterol; blue, second harmonic generation collagen. Sec also FIG. 9.

FIGS. 2A-2O show the transfer of membrane cholesterol from cancer-associated fibroblasts to cancer cells via trogocytosis. (FIG. 2A) Flow cytometry measurement of uptake of CAFs membranes by KPCN349 cells in co-culture. CAFs were labeled with BODIPY-cholesterol and KPCN349 cells with CPD670 and plated at 1:1 ratio for the indicated times. (FIG. 2B) Trogocytosis of CAF membranes by both murine and human PDAC cells co-cultured with BODIPY-cholesterol labeled CAFs over time. The percentage of BODIPY-positive carcinoma cells was enumerated by flow cytometry. Data are represented as mean±SEM. Transwell, PDAC cells and CAFs were cultured for 12 hours separated by 4-micron semi-permeable membrane, 4 min, results assessed 4 minutes after cell mixing CM, 24-hour CAF conditioned media. (FIG. 2C) Trogocytosis of PKH67-labeled CAF membranes in co-cultures with murine PDAC cells. (FIG. 2D) Trogocytosis of CAF membranes is suppressed in the absence of extracellular calcium. Data are represented as mean±SEM. **, p<0.01 (unpaired two-tailed t-test). (FIG. 2E) Representative image of contact-dependent trogocytosis of CAF membranes by murine PDAC cells (the white-colored asterisk indicates CPD670-labeled KPCN349 cell (red) in direct contact with the CAF cell (green, BODIPY-cholesterol). (FIG. 2F) Enumeration of KPCN349 PDAC cells acquiring BODIPY-cholesterol from pre-labeled CAFs in co-cultures. Data acquired from a representative experiment as shown in E. (FIG. 2G) Trogocytic uptake and expression of human HLA on the surface of murine DsRed-tagged KPCN349 cells co-cultured with human CAFs at 1:1 ratio for 8 hours. Flow cytometry enumeration was gated on DsRed-expressing KPCN349 cells. (FIG. 2H) Trogocytosis of CAF membranes by Panc-1 carcinoma cells. Shown, enhanced Z-stacked image of human CAFs expressing actin-RFP. (FIG. 2I) Trogocytosis of CAF membranes labeled with PHK67 by Panc-1 cell is inhibited at 4° C. or in the presence of 3 mM EGTA but not in the presence of 10 ΣM Dynasore, a dynamin inhibitor. Data represent mean fluorescence intensity normalized to vehicle. (FIG. 2J) Masking cell surface phosphatidylserine with recombinant annexin V or bis-Zn-dipicolylamine (Zn-DPA) antagonizes trogocytosis of CAF membranes by KPCN349 cells. CAFs and KPCN349 cells were labeled as in (A). Annexin V and Zn-DPA were added to co-culture media. (FIG. 2K) Apoptosis in LDS of Nsdhl-deficient KPCN349 cells is rescued by co-culture with CAFs, or by addition of 50 μM LDL cholesterol, but not in Transwell co-cultures (CAF/TW), or by in CAF-conditioned media (5% LDS/DMEM for 72 hours, CM). Apoptosis of carcinoma cells was detected by percentage of Annexin-V/FITC positive by flow cytometry. (FIG. 2L) CAFs support long-term growth of pancreatic carcinoma cells. Shown are averaged colony areas in the indicated conditions. (FIG. 2M) Representative PDAC colonies stained with methylene blue. (FIG. 2N, O) Knockdown of LDLR in Panc-1 cells (N) or HDL receptor inhibitor BTL1 (O) cause loss of Panc-1 viability. Co-cultures with human CAFs rescue Panc-1ΔNSDHL cells (CRISPRi-depleted of NSDHL) viability in LDS despite LDLR or HDLR blockade. In all graphs, data are represented as mean±SEM. p-values: *, p<0.05, **<0.01, ***<0.001, ****<0.0001 using unpaired two-tailed t-test, or Mann Whitney test in K. See also FIG. 10.

FIGS. 3A-3F shows high expression of ANO6 in pancreatic adenocarcinoma confers poor survival. (FIG. 3A) Ano gene family transcripts expression visualization by single-cell RNA sequencing of 17,205 single cells isolated from four advanced mouse KPC pancreatic tumors. (FIG. 3B) ANO gene family transcripts expression visualization by single-cell RNA sequencing of 20,929 single cells isolated from five human pancreatic tumors. In A and B, the circle size represents percentage of indicated cells and color intensity represents Z-score normalized expression of the genes. (FIG. 3C) Comparison of overall survival of stage 1-3 PDAC patients from the top and the bottom quartiles of ANO6 protein expression obtained from the NCI Clinical Proteomic Tumor Analysis Consortium (CPTAC) dataset. (FIG. 3D) Comparison of overall survival of stage 1-3 PDAC patients by ANO6 mRNA expression above or below the fragments per kilobase of exon per million mapped fragments (FPKM) cut off 15.36. Data was obtained from the NCI TCGA program. In C and D, p-values by Mantel-Cox log-rank test. (FIG. 3E) ANO6 expression by western blot in protein lysates from fibroblastic cell lines obtained from PDAC tumor (T) or adjacent non-malignant pancreatic tissues (N). (FIG. 3F) Stromal signatures associated with positive (IHC score 2+ and 1+, left) or high (IHC score 2+, right) staining of ANO6 in n=101 pancreatic adenocarcinoma tissue sections. See also FIG. 11.

FIGS. 4A-4J show ANO6 regulates tumor-promoting function of CAFs. (FIG. 4A) Viability of cholesterol auxotroph MIAPaCa-2ΔNSDHL cells (CRISPRi-depleted of NSDHL) cultured for 4 days in 10% LDS alone or in the presence of control CAFs or ANO6-deficient fibroblasts (ANO6-KD, CRISPRi-depleted of ANO6). (FIG. 4B) Viability of cholesterol auxotroph Panc-1ΔNSDHL cells (CRISPRi-depleted of NSDHL) cultured for 4 days in 10% LDS alone or in the presence of control CAFs, ANO6-KD, or ANO6-KD modified with lentiviral vectors to express a full-length murine ANO6-mCherry, or ANO6 with indicated mutations: D409G (constitutively active), D703R (inactive due to calcium-domain mutation), and Y563A (constitutively active). In A,B, data represent fluorescence of cancer cells pre-labeled with Hoechst-33342 relative to co-cultures with control CAFs. Each clement represents individual technical replicate. *, p<0.05, **, p<0.001 (unpaired two-sided t-test). (FIG. 4C) Tumor weights at 5 weeks following orthotopic implantations of 5×105 Panc-1ΔNSDHL cells alone or with ANO6-modified CAFs at 1:3 ratio. (FIG. 4D) Viability of cholesterol auxotroph Panc-1ΔNSDHL cells cultured for 4 days in 10% FBS, 10% LDS alone, or in LDS with CAFs modified with doxycycline-inducible shRNA targeting ANO6 or control non-targeting shRNA. Data represent cumulative results of 3 repeats normalized to control shRNA in the absence of doxycycline. (FIG. 4E) ANO6-deficient fibroblasts show reduced alignment of extracellular matrix fibers in vitro labeled with anti-fibronectin. Color of the fibers represents normalized values of fibers angles. The fiber angle distribution was determined by ‘Orientation J’ plugin in Image J. (FIG. 4F) Percentages of fibronectin fibers aligned at less or equal 15° angles in matrices produced by control CAFs as compared to ANO6-KD CAFs. Graph represents mean±SD. ***, p<0.001 (unpaired two-tailed t-test). (FIG. 4G) Representative Images of CRISPRi-control and ANO6-KD fibroblasts in phase contrast (top) or Nile red (bottom) labeling of lipid droplets. (FIG. 4H) Increased lipid droplets fluorescence (Nile red) in ANO6-null fibroblasts compared to controls CAFs. (FIG. 4I) Expression of indicated protein epitopes in total cellular lysates of CRISPRi-control and ANO6-KD fibroblasts. Bands density relative to control CAF lysates are shown under each lane. (FIG. 4J) Normalized enrichment scores for select (FDR<0.25) Hallmark signatures in ANO6-KD fibroblasts relative to the CRIPSRi-controls. In all graphs, data are represented as mean±SEM. p-values: *, p<0.05, **<0.01, ***<0.001, ****<0.0001 using unpaired two-tailed t-test. Sec also FIG. 12.

FIGS. 5A-5L show synapse-like contacts between cancer cells and CAFs activate Orai calcium channels. (FIG. 5A) Representative images of the fluorescent cytosolic Ca2+ indicator Fluo4 (green) in co-cultures of DsRed-tagged Panc-1 cells (red) and CAFs. Arrow, absence of Fluo4 signal in a lower calcium content in CAFs not contacting cancer cells. (FIG. 5B,C) Quantification of Fluo4 intensity in individual CAFs (B) and Panc-1 cells (C) from 3 independent experiments. TNC, total number of cells; bar, mean±SEM; ns, not significant; **, p<0.01; ****, p<0.0001 as compared with Fluo4 intensity in CAFs or Panc-1 monocultures (one-way ANOVA). (FIG. 5D) Quantification of fluorescence intensity of GCaMP cytosolic Ca2+ reporter in individual CAFs in co-cultures with Panc-1 cells. Data shown are representative of 3 independent experiments. (FIG. 5E) Polarized GCaMP fluorescence at the CAF and Panc-1 synapse-like contact. (FIG. 5F) Quantification of GCaMP mean fluorescence intensity (MFI) at the CAF-Panc-1 interface as compared to the interior of CAFs. TNC, total number of cells; bar, mean±SEM; ****, p<0.0001, one-way ANOVA. (FIG. 5G) GCaMP fluorescence intensity tracing in individual CAFs over time. (FIG. 5H) Orai channel inhibitors BTP2 and zegocractin abrogated Fluo4 fluorescent signal induced by CAF and Panc-1 contact in co-cultures as in (A). Fluo4 intensity in CAFs was measured at 5 minutes after addition of indicated concentrations of inhibitors. Line colors: black, CAFs alone; cyan, CAF cells in co-culture not making contacts with Panc 1 cells; blue, CAFs making membrane contacts with Panc-1 cells. Data are represented as mean±SD. ****, p<0.0001 as compared with CAFs making membrane contacts with Panc-1 cells (unpaired two-sided t-test). (FIG. 5I) Reconstituted (maximum intensity) confocal Z-stack images of CAFs transfected with GFP-MAPPER and ANO6-mCherry prior to co-cultured with Panc-1 cells. Right, graph represents fluorescence intensity distribution within indicated colored boxes. Data are represented as mean±SEM. **, p<0.01, ***, p<0.001 comparing the cell contact box (white) with the cell body (blue) (one-way ANOVA). (FIG. 5J, K) STIM1 (J) and ANO6 (K) fluorescent signal intensity distribution comparing the cell contacts (white box) with the cell body (blue box). CAFs were transfected with YFP-STIM1 (J) or ANO6-mCherry (K) before co-culture with Panc-1 cell. Data are represented as mean±SEM. ***, p<0.001 ****, p<0.0001. (FIG. 5L) Immunofluorescent labeling of endogenous STIM1(green) in CAF and DsRed-tagged Panc-1 cells (red). Graph represents fluorescent signal intensity of STIM1 (mean±SEM) within indicated colored boxes. *, p<0.05; ***, p<0.001 comparing the cell contact box (white) with the cell body (blue) (one-way ANOVA). See also related FIG. 13.

FIGS. 6A-6I show inhibitors of ANO6 block phosphatidylserine externalization in vivo and in vitro. (FIG. 6A) Model of clofazimine bound to mouse ANO6 protein based on cryo-EM structure (PDB 8TAG). ANO6 transmembrane (TM) helices are shown as ribbons. Clofazimine is docked in the cleft between TM1 and TM6. Below the drug binding cleft, the large green spheres represent Ca2+ ions bound within the ion conduction channel. Insert, detailed docking model of clofazimine bound in the ANO6 drug binding cleft. Amino acid residues (stick representation) that are within 4 Angstroms of clofazimine atoms (ball-and-stick representation) are labeled by 1-letter amino acid code and number and colored by clement with carbons shown in light gray. (FIG. 6B) Clofazimine treatment of tumor-bearing KPC mice blocks PtdSer externalization in cytokeratin-positive cancer cells and podoplanin-positive CAFs. PtdSer was detected by 50 μg of KL5c betabody givendelivered IV 5 hours prior to sacrifice. (FIG. 6C) Clofazimine treatment blocks PtdSer externalization in KPC tumors as detected by a PSVuc-794 infrared probe given delivered IV 24 hours prior to sacrifice. In B and C, mice were treated with two daily doses of clofazimine 50 mg/kg or corn oil as vehicle. Data represent mean±SD PtdSer probe intensity; **, p<0.01, ***, p<0.001 (unpaired two-tailed t-test). (FIG. 6D) CRISPRi-knockdown of ANO6, or ANO6 inhibitor niclosamide prevents PtdSer externalization induced by calcium ionophore ionomycin (5 minutes at 10 μM) as assessed by intravital annexin XII staining of CAF membranes. (FIG. 6E) Quantification of total annexin XII fluorescence intensity relative to control CAFs; bar, mean±SD. *, p<0.05, **, p<0.01, ****, p<0.0001 as compared with control CAFs treated with ionomycin (unpaired two-tailed t-test). (FIG. 6F) DsRed-tagged Panc-1 cells (red) induce PtdSer externalization in unlabeled CAFs detected by intravital annexin XII staining (green). Note absence of annexin XII labeling in outlined CAFs in the presence of niclosamide (Niclo), or in CAFs not making membrane contacts with Panc-1 cells (no contact). (FIG. 6G) Quantification of total annexin XII fluorescence intensity in CAFs in co-cultures with Panc-1 cells relative to control CAFs; bar, mean±SD. *, p<0.05, **, p<0.01, ****, p<0.0001 (unpaired two-tailed t-test). (FIG. 6H) Annexin XII fluorescence (green) measuring PtdSer externalization in unlabeled CAFs in co-cultures with DsRed-tagged Panc-1 cells (red) is blocked by the SOCE channel inhibitor BTP2 (10 μM) or the ANO6 inhibitor clofazimine (1 μM). (FIG. 6I) Quantification of total annexin XII fluorescence intensity in CAFs in co-cultureds with Panc-1cells relative to vehicle-treated; bar, mean±SD. *, p<0.05, **, p<0.01 (unpaired two-tailed t-test). See FIG. 14.

FIGS. 7A-7F shows blockade of ANO6 restrains growth and blocks delivery of exogenous lipids to pancreatic tumors. (FIG. 7A) Treatment schemca of mice carrying KPC3 syngencic pancreatic tumor grafts. Animals were given 3 weekly cycles of niclosamide 150 mg/kg/day, clofazimine 50 mg/kg/day, or vehicle followed by intravital imaging and tumor collection. (FIG. 7B, C) Weights of orthotopic syngeneic pancreatic tumors following treatment with oral ANO6 inhibitors niclosamide (B) or clofazimine (C) at 3 weeks post implantation. Data are represented as mean±SD. ***, p<0.001 compared with vehicle-treated mice (unpaired two-tailed t-test). (FIG. 7D) Intravital imaging quantification of intratumoral and peri-tumoral BODIPY-cholesterol fluorescence in DsRed-tagged KPC3 tumors at 10, 16 and 19 hours following intravenous administration of labeled LDL. Data are represented as mean±SD of ratios of intratumoral to peri-tumoral BODIPY-cholesterol fluorescence. *, p<0.05 (FIG. 7E) Representative intravital images of vesicles containing BODIPY-cholesterol within orthotopic DsRed-tagged KPC3 tumors treated with niclosamide or vehicle. Scale bars, 30 μm. (FIG. 7F) Quantification of intratumoral vesicles as in (E). Cumulative data from 2 independent imaging experiments are represented as mean±SD of vesicle volumes enumerated from multiple tumor areas in 2-3 mice at each time point, each clement represents vesicles volume in a tumor area. *, p<0.05 (unpaired two-tailed t-test). See also FIG. 14.**

FIGS. 8A-8L shows CAF-expressed ANO6 induces the dysfunction of the cytotoxic T lymphocytes in PDAC. (FIG. 8A) Flow cytometry reveals the percentage of pancreatic intratumoral CD3+CD8+ CTLs, NK cells, conventional type I dendritic cells, and CD4+FoxP3+ regulatory T cells from orthotopic KPC3 pancreatic tumors in mice treated with clofazimine or vehicle as described in FIG. 6C. (FIG. 8B) Flow cytometry profiles of Filipin III staining corresponding to cholesterol levels in the intratumoral CTLs from mice treated with clofazimine or vehicle as described in FIG. 6C. (FIG. 8C) Quantification of Filipin III mean fluorescence intensity (MFI) related to FIG. 6B. (FIG. 8D) Expression of activity (CD69) and exhaustion (PD-1 and TIM3) markers on the intratumoral CD3+CD8+ CTLs isolated from mice treated with clofazimine or vehicle as described in FIG. 6C. (FIG. 8E) Effect of clofazimine on intracellular interferon-γ and granzyme B expression in intratumoral CTLs. (FIG. 8F) CAFs membrane trogocytosis by OT-1 CTLs. DiD-labeled CAFs were co-cultured with OT-1 CTLs at a 1:5 ratio, respectively, in the presence or absence of niclosamide, clofazimine, annexin V, anti-TIM3 and 25HC for 12 h. (FIG. 8G) Enumeration of membrane cholesterol in OT-1 CTLs by Filipin III following co-cultures with CAFs pre-treated as in (F). (FIG. 8H) Intracellular interferon-γ in OT-1 CTLs following co-culture with CAFs treatments as in (E). (FIG. 8I) CAFs membrane trogocytosis by OT-1 CTLs. DiD-labeled CAF Ctrl or ANO6-KD were co-cultured with OT-1 CTLs at a 1:5 ratio for 12 hours. (FIG. 8J) Enumeration of membrane cholesterol in OT-1 CTLs by Filipin III following co-cultures with Ctrl or ANO6-KD CAFs Ctrl or ANO6-KD. (FIG. 8K) Intracellular interferon-γ in OT-1 CTLs following co-culture with CAFs. (FIG. 8L) Activation of OT-1 CTLs measured by MC38OVA-luc cells lysis following co-culture with Ctrl or ANO6-KD CAFs. In all graphs, data are presented as mean±SEM from n=5 tumors per treatment or n=4 replicates for each in vitro condition; p-values were calculated by unpaired two-tailed t test: *, p<0.05; **, p<0.01; ***, p<0.001; ****, p<0.0001. See also FIGS. 15 and 16.

FIGS. 9A-9C show the delivery of low-density lipoprotein particles to pancreatic cancer cells is mediated by cancer-associated fibroblasts. (FIG. 9A) Representative images of BODIPY-cholesterol fluorescence (green) in pancreatic tumor and liver cryosections. Tumor-bearing KPC mice were injected intravenously with 100 μL of BODIPY-cholesterol labeled human LDL and sacrificed at indicated times post injection. (FIG. 9B) Representative Oil Red staining of lipid droplets in murine pancreatic tumors from 8-week-old KPC mice maintained on regular diet. Note accumulation of lipid droplets around vessels (top) and in the stroma (bottom). (FIG. 9C) Summary table of 3 independent experiments enumerating the percentage of cells in pancreatic orthotopic tumors positive for LDL signal. GFP-tagged KPCN349 cells implanted to the pancreas were allowed to grow for 2 weeks. Tumor-bearing mice were injected with 100 μL of DiI-labeled human LDL (Invitrogen) and sacrificed at 90 minutes post injection. Single-cell suspensions were analyzed by flow cytometry using indicated surface markers. Percentage of DiI-positive cells was enumerated relative to the total number of lineage maker-positive cells.

FIGS. 10A-10D show the transfer of membrane cholesterol from cancer-associated fibroblasts to cancer cells via trogocytosis. (FIG. 10A) Trogocytosis of CAF membranes by Panc-1 cells is inhibited at low temperatures (+4° C.) and in the absence of Ca2+ and Mg2+ in the media. CAFs labeled with PKH67 and Panc-1 cells labeled with CMCMR were co-cultured for indicated duration of time and conditions; 5% FBS/DMEM media conditioned for 2-days with PKH67-labeled CAF was used as a negative control. Trogocytosis of CAF membranes by Panc-1 cells in co-culture was measured by flow cytometry. (FIG. 10B) Electron microscopy of CAF membrane trogocytosis by PDAC cells. CAFs were surface biotinylated with sulfo-NHS-Biotin followed by surface labeling with colloidal gold-conjugated streptavidin and incubated for 4 hours with unlabeled KPCN349 cells. Note gold-positive CAF membrane projections (center and right insert) engulfed in cancer cell endosomes and electron-dense lysosomes (left insert). (FIG. 10C) Trogocytosis of CAF membranes is partially suppressed in the presence of cytoskeleton inhibitors lantrinculin A (LanA) and cytochalasin D (CytoD). (FIG. 10D) Representative flow cytometry histograms of BODIPY-cholesterol fluorescence of KPCN349 cells labeled with CDP670 as in FIG. 2A. To mask cell surface phosphatidylserine, recombinant annexin V or bis-Zn-dipicolylamine (Zn-DPA) were added to co-culture media. Quantified data are presented in FIG. 2J.

FIGS. 11A-11C show analyses of ANO6 expression in human cancers from TCGA and in PDAC surgical samples. (FIG. 11A) Kaplan-Meier analyses of overall survival of TCGA Pan-Cancer (left), stage 1-3 breast cancer (middle), and cervical squamous cell carcinoma by ANO6 mRNA expression above or below the median value of fragments per kilobase of exon per million mapped fragments (FPKM). Data was obtained from the NCI TCGA program; p-values by Mantel-Cox log-rank test. (FIG. 11B) Unsupervised clustering of differentially expressed genes across stroma comparing ANO6 high (IHC score 2+) versus ANO6 low (IHC score 1+/0) in human PDAC (n=101). HThe heat map represents Z-score normalized expression values. ANO6 IHC scores in stroma and tumor are color coded. Batch, to account for the IHC staining batch effect; PurIST and Molecular gradient are PDAC classes based on bulk RNA expression. (FIG. 11C) Representative images of IHC labeling of ANO6 in FFPE tissue sections of PDAC surgical samples

FIGS. 12A-12M show ANO6 regulates tumor-promoting function of CAFs. (FIG. 12A) Western blot demonstrating loss of ANO6 in CRISPRi-modified CAF1 cells. (FIG. 12B) Viability of cholesterol auxotroph Panc-1ΔNSDHL cancer cells in lipid-depleted serum (5% LDS) co-cultured with control CAF1 cells or ANO6-KD fibroblasts. Data are presented as mean±SEM of ratio of dead (SYTOX Blue-positive) and live (DsRed-positive) Panc-1 cells. Fluorescent images were acquired on ImageXpress (Molecular Devices) microscope and analyzed using MetaXpress software. (FIG. 12C) Total annexin XII fluorescence intensity measured by microscopy of viable CAFs pulsed for 5 minutes with 1 μM ionomycin in DMEM containing Ca2+/Mg2+. CAF1 carrying non-targeting sgRNA were used as controls. ANO6-KD are CRIPSRi-depleted of ANO6. The ANO6-KD were modified with lentiviral vectors to express a full-length murine ANO6-mCherry (calcium-responsive), or ANO6 with indicated mutations: D409G (constitutively active), D703R (inactive due to calcium-domain mutation), and Y563A (constitutively active). (FIG. 12D) Total annexin XII fluorescence intensity in CAF1 cells as in C, except without addition of ionomycin. Data of individual images obtained in two independent experiments are represented as mean±SD. (FIG. 12E) Images of dissected pancreatic tumors at 5 weeks following orthotopic implantations of 5×105 of Panc-1sgControl or Panc-1ΔNSDHL cells. (FIG. 12F) Weights of cholesterol auxotroph NSDHL-depleted tumors as compared to sgControl-modified Panc-1 cells. (FIG. 12G) Western blot demonstrating ANO6 depletion in CAF1 cells by Doxycycline-inducible shRNA (pLKO-Dox) following 72 hours of treatment with 1 μg/ml doxycycline. (FIG. 12H) Growth rates of CAF1 sgControl and sgANO6 CRISPRi-depleted CAF1 cells using xCELLigence. (FIG. 12I) Growth rates of CAF1 cells carrying Dox-inducible shControl and shANO6 using xCELLigence. (FIG. 12J) Quantification of cell surface areas of CAF1 sgControl and sgANO6 CRISPRi-depleted CAF1 cells. (FIG. 12K) Lipid droplets fluorescence intensity following Nile Red labeling of CAF1 carrying non-targeting sgRNA as control, CAF1 modified to express sgANO6 (ANO6-KD), or ANO6-KD reconstituted with lentiviral expression vectors of full-length murine ANO6-mCherry, or ANO6-Cherry with indicated mutations: D703R (inactive due to calcium-domain mutation), and Y563A (constitutively active). (FIG. 12L) Blockade of ANO6 with Niclosamide suppresses signaling. Expression of indicated protein epitopes in total cellular lysates of unmodified CAF1 fibroblasts. Bands densitiesy relative to vehicle control in CAF lysates are shown under each lane. (FIG. 12M) Depletion of ANO6 with doxycycline-inducible Tet-pLKO-puro shRNA suppresses signaling as compared to non-targeting control shRNA. Expression of indicated protein epitopes in total cellular lysates of modified CAF1 fibroblasts. Bands densitiesy relative to control non-targeting shRNA in CAF lysates are shown under each lane. In all graphs, data are represented as mean±SD. **, p<0.01; **, p<0.001; ****, p<0.0001 (unpaired two-tailed t-test).

FIGS. 13A-13K show an influx of extracellular calcium via Orai channels activates ANO6 in CAFs. (FIG. 13A) Measurements of Ca2+ influx in CAFs using Fura2 fluorescent calcium indicator. CAFs were loaded with Fura2-AM for 30 mins and treated with vehicle, zegocractin 5 μM, or BTP2 1 μM for 10 mins. Thapsigargin 2 μM (Tg) was added to deplete ER Ca2+ stores in the absence of extracellular Ca2+. Once Fura2 signal returned to the baseline, extracellular Ca2+ was elevated to 1 mM. (FIG. 13B) Estimated SOCE in CAFs. The magnitude of SOCE was assessed by subtracting basal F340/F380 from the maximum F340/F380 in CAF cells treated with vehicle, CM4620, or BTP2. (FIG. 13C) Total annexin XII fluorescence intensity measured by microscopy of viable CAFs pulsed for 5 minutes with 1 μM ionomycin in DMEM containing Ca2+/Mg2+ or with added cell-impermeable 2 mM EGTA. Right, representative images of annexin XII labeling. (FIG. 13D) Representative images of MAPPER expression at the plasma membranes of single Panc-1 cells (left) or at the interface of Panc-1 cells making a synapse-like contact (right). Images are reconstituted (maximum intensity) confocal Z-stacks of DsRed-tagged Panc-1 cells transfected with MAPPER-GFP. (FIG. 13F) Mean fluorescence intensity (MFI) of MAPPER-GFP. (FIG. 13G) Absence of polarized GCaMP fluorescence (green) at the CAF-CAF contacts. Graph represents quantification of GCaMP mean fluorescence intensity (MFI) at the CAF-CAF interface as compared to the interior of CAFs. (FIG. 13H) Representative images of absence of polarization of STIM1-YFP at the CAF-CAF contact sites. Graph represents quantification of STIM1-YFP mean fluorescence intensity (MFI) at the CAF-CAF interface as compared to the interior of CAFs. (FIG. 13I) Representative images of absence of polarization of the endogenous STIM1 at the CAF-CAF contact sites. Graph represents quantification of STIM1-YFP mean fluorescence intensity (MFI) at the CAF-CAF interface as compared to the interior of CAFs. (FIG. 13J) Representative images of the endogenous STIM1 polarization at the Panc-1/Panc-1 contact sites. Graph represents quantification of anti-STIM1 mean fluorescence intensity (MFI) at the interface as compared to the interior of Panc-1 cells. (FIG. 13K) Representative images of the absence of polarization of the transfected ANO6-mCherry at the CAF-CAF contact sites. Graph represents mean fluorescence intensity (MFI) of ANO6 at the interface as compared to the interior of CAFs. In D, E, G, H and K, CAFs were transfected with MAPPER-GFP, GCaMP, STIM1-YFP, or ANO6-mCherry as indicated. Fluorescence distribution near the cell contact site (white) or away from the contact site (blue) is shown by boxes. TNC, total number of cells. Data of individual images obtained in at least two independent experiments are represented as mean±SEM; ns, not significant; ****, p<0.0001, one-way ANOVA.

FIGS. 14A-14D shows inhibitors of ANO6 block phosphatidylserine externalization and antagonize tumor growth. (FIG. 14A, B) Representative images of KPC PDAC tumors following 2 daily oral doses of 50 mg/kg of clofazimine. Externalized phosphatidylserine in tumors was detected by KL5c betabody (50 μg in saline) injected IV 5 hours prior to sacrifice. Cryosections were counterstained with cytokeratin antibody to highlight cancer cells (A) or podoplanin antibody for CAFs (B). (FIG. 14C, D) Images of individual pancreatic tumors collected at 3 weeks post implantation of 105 of KPC3 PDAC cells to pancreatic tails. Animals were treated with niclosamide (C) or clofazimine (D) as indicated in FIG. 7.

FIGS. 15A-15F show an assessment of CAF-expressed ANO6 on the status of the cytotoxic T lymphocytes. (FIG. 15A) Gating strategy for assessment of intratumoral and splenic immune cells (equally applied to all samples). Single cells in dissociated suspensions of pancreatic tumors were identified using combined SSC and FSC filtering to exclude debris, apoptotic cells and cell doublets as outlined with gates P1-P3. Both CD45− and CD45+ cells were present in the single cell populations. The analyses of immune cells were done exclusively on CD45+ population. “Backgating” was used to confirm that the identified immune cell subtypes were associated with non-apoptotic single-cell populations. (FIG. 15B) Clofazimine has no effect on preponderance of immune subsets in the spleens of tumor-bearing mice. (FIG. 15C) Clofazimine does not change the percentage of intratumoral CD11b+F4/80+ macrophages, LY6G+ granulocytes and LY6C+ MDSCs. (FIG. 15D) Flow cytometry profiles of Filipin III staining corresponding to cholesterol levels in the splenic CD3+CD8+ CTLs from mice treated with clofazimine or vehicle as described in FIG. 6C. (FIG. 15E) Clofazimine has no effect on expression of activity (CD69) and exhaustion (PD-1 and TIM3) markers, or intracellular interferon-γ in splenic CD3+CD8+ CTLs isolated from mice treated with clofazimine or vehicle as described in FIG. 6C. (FIG. 15F) Flow cytometry profiles of activity (CD69) and exhaustion (PD-1 and TIM3) markers on the intratumoral CTLs from mice treated with clofazimine or vehicle as described in FIG. 7A. ***

FIGS. 16A-16H show an assessment of CAF-expressed ANO6 on the status of the cytotoxic T lymphocytes in vitro. (FIG. 16A) Representative images of fluorescent cytosolic Ca2+ indicator Fluo4 (green) in monocultures of OT-I CTLs (left), CAF1 cells (middle) or co-cultures of OT-I CTLs with CAF1 cells (right). (FIG. 16B) Quantification of Fluo4 intensity in individual cells from 2 independent experiments. TNC, total number of cells; bar, mean±SEM; ns, not significant; ****, p<0.0001 as compared with Fluo4 intensity in CAFs monoculture (one-way ANOVA). (FIG. 16C) Flow cytometry dot plots of DiD uptake by OT-1 CTLs cultured for 12 hours either alone or with DiD-labeled CAFs at a 1:5 ratio in the presence of niclosamide, clofazimine, annexin V, anti-TIM3 and 25HC, respectively. (FIG. 16D) Flow cytometry profiles of Filipin III of OT-1 CTLs cultured for 12 hours either alone or with DiD-labeled CAFs at a 1:5 ratio in the presence of niclosamide, clofazimine, annexin V, anti-TIM3 and 25HC, respectively. (FIG. 16E) Flow cytometry dot plots of intracellular IFN-γ in OT-1 CTLs co-cultured for 12 hours either alone or with DiD-labeled CAFs at a 1:5 ratio in the presence of niclosamide, clofazimine, annexin V, anti-TIM3 and 25HC, respectively. (FIG. 16F) Flow cytometry dot plots of DiD uptake by OT-1 CTLs cultured for 12 hours either alone or with DiD-labeled control CAFs or ANO6-KD CAFs. (FIG. 16G) Flow cytometry profiles of Filipin III in OT-1 CTLs cultured for 12 hours either alone or with control CAFs or ANO6-KD CAFs. (FIG. 16H) Flow cytometry contour density plots of intracellular IFN-γ in OT-1 CTLs cultured for 12 hours either alone or with control CAFs or ANO6-KD CAFs. In all graphs, data are presented as mean±SEM from n=5 tumors per treatment or n=4 replicates for each in vitro condition; p-values were calculated by unpaired two-tailed t test: *, p<0.05; **, p<0.01; ***, p<0.001; ****, p<0.0001.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and discussed and a number of modifications that can be made to a number of molecules within the composition are discussed, each and every combination and permutation of the composition and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an OOrai channel inhibitor” includes a plurality of such inhibitors, reference to “a calcium channel inhibitor” is a reference to one or more calcium channel inhibitors and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of the cancer. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient”. In some aspects, a “subject in need thereof” has been diagnosed with cancer prior to administering the calcium channel inhibitor to the subject. In some aspects, a subject in need thereof has been diagnosed with lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma prior to administering the calcium channel inhibitor to the subject.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

Disclosed are compositions comprising a calcium channel inhibitor. In some aspects, the calcium channel inhibitor can be an Orai channel inhibitor. Thus, disclosed are compositions comprising an Orai channel inhibitor. Also disclosed are compositions comprising an Orai channel inhibitor and an anti-cancer agent.

In some instances, the compositions can further comprise a pharmaceutically acceptable carrier. Thus, also disclosed are pharmaceutical compositions comprising an Orai channel inhibitor; an anti-cancer agent; and a pharmaceutically acceptable carrier.

In some aspects, the Orai channel inhibitor can be an Orai1, Orai2, or Orai3 channel inhibitor. In some aspects, the Orai channel inhibitor is CM4620, BTP2, Synta-66, RO2959, GSK-7975A, JPIII, N-MeDCPA, or any combination thereof.

In some aspects, the anti-cancer agent can be an antimetabolite, a microtubule targeting agent, an alkylating agent, a topoisomerase inhibitor, immune checkpoint blockade inhibitors, DNA targeted chemotherapeutic agent, or a combination thereof. In some aspects, an antimetabolite can be, but is not limited to, methotrexate, mercaptopurine, cytarabine, hydroxyurea, thioguanine, mitomycin. In some aspects, a microtubule targeting agent can be, but is not limited to, taxol, vinblastine, vincristine, vinorelbine, paclitaxel, or docetaxel. In some aspects, an alkylating agent can be, but is not limited to, cyclophosphamide, nitrosoureas, ifosfamide, cisplatin, carboplatin, dacarbazine, or procarbizine. In some aspects, a topoisomerase inhibitor can be, but is not limited to, ctoposides, camptothecins, doxorubicin, idarubicin, or daunorubicin. In some aspects, a DNA targeted chemotherapeutic agent can be, but is not limited to, bleomycin, dactinomycin, plicamycin, mitoxantrone, asparaginase. In some aspects, an immune checkpoint blockade inhibitor can be, but is not limited to, antibodies blocking PD1 and PD-IL, TIM3, LAG3.

In some aspects, the disclosed compositions can further comprise an ANO6 inhibitor. In some aspects, an ANO6 inhibitor can be, but is not limited to, niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof. Disclosed are compositions comprising an Orai channel inhibitor and an ANO6 inhibitor. Also disclosed are compositions comprising an Orai channel inhibitor, an anti-cancer agent, and an ANO6 inhibitor.

By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed calcium chloride inhibitors can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. Orai inhibitor) in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising an Orai channel inhibitor and a cationic liposome can be administered to the blood, to a target organ, or inhaled into the respiratory tract to target cells of the respiratory tract. For example, a composition comprising an Orai channel inhibitor described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising a combination of an OOrai channel inhibitor, an anti-cancer agent, and a pharmaceutically acceptable carrier, buffer, or diluent. In various aspects, the Orai channel inhibitor and/or anti-cancer agent of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the Orai channel inhibitors and/or anti-cancer agents described herein and can also include a carrier such as a pharmaceutically acceptable carrier. For example, disclosed are pharmaceutical compositions, comprising the Orai channel inhibitor and/or anti-cancer agent disclosed herein, and a pharmaceutically acceptable carrier. In one aspect, disclosed are pharmaceutical compositions comprising calcium channel inhibitors. That is, a pharmaceutical composition can be provided comprising a therapeutically effective amount of at least one Orai channel inhibitor, an anti-cancer agent, and a pharmaceutically acceptable carrier.

In certain aspects, the disclosed pharmaceutical compositions comprise the disclosed Orai channel inhibitors and anti-cancer agents as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compositions described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The compositions described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed compositions in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the case in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their case of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed Orai channel inhibitor and/or anti-cancer agent as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed Orai channel inhibitor, and/or pharmaceutically acceptable salts thereof, can also be prepared in powder or liquid concentrate form.

The exact dosage and frequency of administration depends on the particular disclosed Orai channel inhibitor, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### C. Methods

Disclosed are methods of using one or more of the disclosed compositions

comprising a calcium channel inhibitor, for example, an Orai channel inhibitor.

### 1. Methods of Treating Cancer

Disclosed are methods of treating cancer in a subject comprising administering to the subject a calcium channel inhibitor. Disclosed are methods of treating cancer, comprising administering to a subject in need thereof a calcium channel inhibitor. In some aspects, the subject is human. In some aspects, a subject in need thereof has been diagnosed with cancer prior to administering the calcium channel inhibitor to the subject. In some aspects, a subject in need thereof has been diagnosed with lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma prior to administering the calcium channel inhibitor to the subject.

In some aspects, the calcium channel inhibitor targets cancer-associated fibroblasts (CAFs). Thus, in some aspects, the calcium channel inhibitor does not target a cancer cell.

In some aspects, the calcium channel inhibitor can be an Orai channel inhibitor. In some aspects, the Orai channel inhibitor can be an Orai1, Orai2, or Orai3 channel inhibitor. In some aspects, the Orai channel inhibitor is CM4620, BTP2, Synta-66, RO2959, GSK-7975A, JPIII, N-MeDCPA, or any combination thereof.

In some aspects, disclose are methods of treating cancer, comprising administering to a subject in need thereof a calcium channel inhibitor wherein one or more biochemical pathways, proteins, and/or enzymes are altered in the subject after administration of a calcium channel inhibitor. Disclosed are methods of treating cancer, comprising administering to a subject in need thereof a calcium channel inhibitor, wherein Ca+ levels in the subject are decreased after administration of a calcium channel inhibitor. Disclosed are methods of treating cancer, comprising administering to a subject in need thereof a calcium channel inhibitor, wherein phospholipid scramblase anoctamin 6 (ANO6) activity in the subject is decreased after administration of a calcium channel inhibitor. Disclosed are methods of treating cancer, comprising administering to a subject in need thereof a calcium channel inhibitor, wherein ANO6 expression is not altered, just activity after administration of a calcium channel inhibitor. Disclosed are methods of treating cancer, comprising administering to a subject in need thereof a calcium channel inhibitor, wherein phosphatidylserine (PtdSer) externalization in the subject is decreased after administration of a calcium channel inhibitor. In some aspects, each of Ca+ levels, ANO6 activity, and PtdSer externalization can be measured using techniques well known in the art.

In some aspects, CAFs transfer exogenous cholesterol and other nutrients via direct transfer of membranous structures via trogocytosis. In some aspects, trogocytosis is the dominant means by which some cancer cells, such as PDAC cells, derive their lipids. In some aspects, decreased lipids can result in cancer cell death. Thus, in some aspects, administering a calcium channel inhibitor to a subject results in a decrease in trogocytosis between CAFs and cancer cells in the subject. In some aspects, the decrease in Trogocytosis prevents transfer of lipids from CAFs to cancer cells which can lead to cell death of the cancer cells.

In some aspects, trogocytosis is regulated by calcium, ANO6, and PtdSer, which can all be decreased in response to administering a calcium channel inhibitor. Therefore, a decrease in calcium, ANO6, and PtdSer externalization can result in a decrease in trogocytosis.

In some aspects, cholesterol can be transferred by CAFs into cytotoxic T cells (CTLs) which causes their exhaustion (e.g. immune suppression). In some aspects, the immunosuppressive effect of CAFs against cytotoxic T cells is attenuated in a subject administered a calcium channel inhibitor. Thus, in some aspects, inhibition of ANO6 not only decreases trogocytosis but also attenuates the immunosuppressive effect of CAFs against CTLs.

In some aspects, disclosed are methods of treating cancer, comprising administering to a subject in need thereof a calcium channel inhibitor, wherein delivery of exogenous cholesterol to cancer cells is disrupted in the subject. Because cancer cells can rely on lipids, in some aspects, disrupting delivery of cholesterol can help treat the cancer.

As discussed throughout, a decrease in ANO6 can result in decreased trogocytosis between CAFs and cancer cells in a subject administered a calcium channel inhibitor. Therefore, in some aspects, Disclosed are methods of treating cancer, comprising administering to a subject in need thereof a calcium channel inhibitor, further comprising administering an ANO6 inhibitor. In some aspects, an ANO6 inhibitor can be, but is not limited to, niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

In some aspects, the anti-cancer agent can be an antimetabolite, a microtubule targeting agent, an alkylating agent, a topoisomerase inhibitor, immune checkpoint blockade inhibitors, DNA targeted chemotherapeutic agent, or a combination thereof. In some aspects, an antimetabolite can be, but is not limited to, methotrexate, mercaptopurine, cytarabine, hydroxyurea, thioguanine, mitomycin. In some aspects, a microtubule targeting agent can be, but is not limited to, taxol, vinblastine, vincristine, vinorelbine, paclitaxel, or docetaxel. In some aspects, an alkylating agent can be, but is not limited to, cyclophosphamide, nitrosoureas, ifosfamide, cisplatin, carboplatin, dacarbazine, or procarbizine. In some aspects, a topoisomerase inhibitor can be, but is not limited to, etoposides, camptothecins, doxorubicin, idarubicin, or daunorubicin. In some aspects, a DNA targeted chemotherapeutic agent can be, but is not limited to, bleomycin, dactinomycin, plicamycin, mitoxantrone, asparaginase. In some aspects, an immune checkpoint blockade inhibitor can be, but is not limited to, antibodies blocking PD1 and PD-IL, TIM3, LAG3.

In some aspects, the cancer being treated can be, but is not limited to, lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma. Thus, in some aspects, the disclosed cancer cells herein can be a cell from any of the disclosed cancers. In some aspects, the disclosed cancer cells can be a cell from any solid cancer, leukemia, or lymphoma.

In some aspects, the disclosed methods comprise administering a pharmaceutical composition comprising an Orai channel inhibitor; an anti-cancer agent; and a pharmaceutically acceptable carrier.

### 2. Method of Decreasing ANO6 Activity in Cancer-Associated Fibroblasts (CAFs)

Disclosed are methods of decreasing ANO6 activity in cancer-associated fibroblasts (CAFs) comprising contacting a CAF with a calcium channel inhibitor. In some aspects, ANO6 expression is not altered, just activity.

In some aspects, the methods can occur in vitro or in vivo. When performed in vivo, the methods comprise administering a calcium channel inhibitor to a subject.

In some aspects, ANO6 is highly expressed in cancer cells, such as PDAC cells, and CAFs and plays a role in Trogocytosis between cancer cells and CAFs. Thus, in some aspects, ANO6 inhibition can starve PDAC tumor cells of lipids by decreasing trogocytosis between PDAC cells and CAFs.

In some aspects, the calcium channel inhibitor targets cancer-associated fibroblasts (CAFs). Thus, in some aspects, the calcium channel inhibitor does not target a cancer cell.

In some aspects, the calcium channel inhibitor can be an Orai channel inhibitor. In some aspects, the Orai channel inhibitor can be an Orai1, Orai2, or Orai3 channel inhibitor. In some aspects, the Orai channel inhibitor is CM4620, BTP2, Synta-66, RO2959,GSK-7975A, JPIII, N-MeDCPA, or any combination thereof.

In some aspects, the calcium channel inhibitor reduces Ca+ which is necessary for ANO6 activity, thereby decreasing ANO6 activity in the CAF. Thus, in some aspects, Ca+ levels in the subject are decreased.

In some aspects, administering a calcium channel inhibitor to a subject results in a decrease in trogocytosis between CAFs and cancer cells in the subject. In some aspects, the decrease in trogocytosis prevents transfer of lipids from CAFs to cancer cells which can lead to cell death of the cancer cells.

In some aspects, inhibition of ANO6 (e.g. decreasing ANO6 activity) not only decreases trogocytosis but also attenuates the immunosuppressive effect of CAFs against CTLs.

In some aspects, the disclosed methods further comprise contacting a CAF with an ANO6 inhibitor. In some aspects, an ANO6 inhibitor can be, but is not limited to, niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

In some aspects, the disclosed methods further comprise administering an anti-cancer treatment. In some aspects, the anti-cancer agent can be an antimetabolite, a microtubule targeting agent, an alkylating agent, a topoisomerase inhibitor, immune checkpoint blockade inhibitors, DNA targeted chemotherapeutic agent, or a combination thereof. In some aspects, an antimetabolite can be, but is not limited to, methotrexate, mercaptopurine, cytarabine, hydroxyurea, thioguanine, mitomycin. In some aspects, a microtubule targeting agent can be, but is not limited to, taxol, vinblastine, vincristine, vinorelbine, paclitaxel, or docetaxel. In some aspects, an alkylating agent can be, but is not limited to, cyclophosphamide, nitrosoureas, ifosfamide, cisplatin, carboplatin, dacarbazine, or procarbizine. In some aspects, a topoisomerase inhibitor can be, but is not limited to, etoposides, camptothecins, doxorubicin, idarubicin, or daunorubicin. In some aspects, a DNA targeted chemotherapeutic agent can be, but is not limited to, bleomycin, dactinomycin, plicamycin, mitoxantrone, asparaginase. In some aspects, an immune checkpoint blockade inhibitor can be, but is not limited to, antibodies blocking PD1 and PD-IL, TIM3, LAG3.

In some aspects, the disclosed methods comprise administering a pharmaceutical composition comprising an Orai channel inhibitor; an anti-cancer agent; and a pharmaceutically acceptable carrier.

### 3. Method of Decreasing ANO6 Activity in a Cancer-Associated Fibroblast (CAF) in a Xubject

Disclosed are methods of decreasing Ano6 activity in a cancer-associated fibroblast (CAF) in a subject, comprising administering to the subject a calcium channel inhibitor, wherein the calcium channel inhibitor reduces Ca+ which is necessary for Ano6 activity, thereby decreasing Ano6 activity in a CAF in the subject. In some aspects, ANO6 expression is not altered, just activity.

In some aspects, the calcium channel inhibitor is an Orai channel inhibitor. In some aspects, the Orai channel inhibitor is CM4620, BTP2, Synta-66, RO2959, GSK-7975A, JPIII, N-MeDCPA or any combination thereof.

In some aspects, the method further comprises administering to the subject an Ano6 inhibitor. In some aspects, the Ano6 inhibitor is niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

In some aspects, disclosed are methods of decreasing Ano6 activity in a cancer-associated fibroblast (CAF) in a subject, comprising administering to the subject a calcium channel inhibitor, wherein the calcium channel inhibitor reduces Ca+ which is necessary for Ano6 activity, thereby decreasing Ano6 activity in a CAF in the subject, wherein trogocytosis between cancer activated fibroblasts (CAFs) and cancer cells in the subject is decreased.

In some aspects, ANO6 is highly expressed in cancer cells, such as PDAC cells, and CAFs and plays a role in Trogocytosis between cancer cells and CAFs. Thus, in some aspects, ANO6 inhibition can starve PDAC tumor cells of lipids by decreasing trogocytosis between PDAC cells and CAFs.

In some aspects, the calcium channel inhibitor targets cancer-associated fibroblasts (CAFs). Thus, in some aspects, the calcium channel inhibitor does not target a cancer cell.

In some aspects, the calcium channel inhibitor can be an Orai channel inhibitor. In some aspects, the Orai channel inhibitor can be an Orai1, Orai2, or Orai3 channel inhibitor. In some aspects, the Orai channel inhibitor is CM4620, BTP2, Synta-66, RO2959, GSK-7975A, JPIII, N-MeDCPA, or any combination thereof.

In some aspects, the calcium channel inhibitor reduces Ca+ which is necessary for ANO6 activity. Thus, in some aspects, Ca+ levels in the subject are decreased.

In some aspects, administering a calcium channel inhibitor to a subject results in a decrease in trogocytosis between CAFs and cancer cells in the subject. In some aspects, the decrease in trogocytosis prevents transfer of lipids from CAFs to cancer cells which can lead to cell death of the cancer cells.

In some aspects, inhibition of ANO6 (e.g. decreasing ANO6 activity) not only decreases trogocytosis but also attenuates the immunosuppressive effect of CAFs against CTLs.

In some aspects, the disclosed methods further comprise administering an ANO6inhibitor. In some aspects, an ANO6 inhibitor can be, but is not limited to, niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

In some aspects, the disclosed methods further comprise administering an anti-cancer treatment. In some aspects, the anti-cancer agent can be an antimetabolite, a microtubule targeting agent, an alkylating agent, a topoisomerase inhibitor, immune checkpoint blockade inhibitors, DNA targeted chemotherapeutic agent, or a combination thereof. In some aspects, an antimetabolite can be, but is not limited to, methotrexate, mercaptopurine, cytarabine, hydroxyurea, thioguanine, mitomycin. In some aspects, a microtubule targeting agent can be, but is not limited to, taxol, vinblastine, vincristine, vinorelbine, paclitaxel, or docetaxel. In some aspects, an alkylating agent can be, but is not limited to, cyclophosphamide, nitrosoureas, ifosfamide, cisplatin, carboplatin, dacarbazine, or procarbizine. In some aspects, a topoisomerase inhibitor can be, but is not limited to, etoposides, camptothecins, doxorubicin, idarubicin, or daunorubicin. In some aspects, a DNA targeted chemotherapeutic agent can be, but is not limited to, bleomycin, dactinomycin, plicamycin, mitoxantrone, asparaginase. In some aspects, an immune checkpoint blockade inhibitor can be, but is not limited to, antibodies blocking PD1 and PD-1L, TIM3, LAG3.

In some aspects, the disclosed methods comprise administering a pharmaceutical composition comprising an Orai channel inhibitor; an anti-cancer agent; and a pharmaceutically acceptable carrier.

### D. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising compositions comprising an Orai channel inhibitor, an anti-cancer agent, and a pharmaceutically acceptable carrier. The kits also can contain each individual component of the disclosed compositions so that they can be delivered alone or as a single pharmaceutical composition.

### Examples

### A. Example 1—Trogocytosis of Cancer-Associated Fibroblasts Promotes Pancreatic Cancer Growth and Immune Suppression Via Phospholipid Scramblase Anoctamin 6 (ANO6)

### 1. Introduction

Cancer cells are characterized by an increased requirement for lipids to sustain their growth and to promote membrane-based receptor signaling. As de novo lipid synthesis consumes energy and oxygen, most aggressive human cancers resort to metabolic parasitism, i.e., uptake of cholesterol and other lipids from exogenous sources. While tumors normally acquire lipids and other nutrients from the bloodstream, the PDAC tumor mass is remarkable in its lack of functional intratumoral blood vessels, consisting predominantly of dense connective tissue populated by cancer associated fibroblasts (CAFs). How the pancreatic cancer cells obtain their essential nutrients, especially membranous lipids such as cholesterol, has thus been mysterious but of high interest, as determining how PDAC cells acquire exogenous lipids could facilitate a potential therapeutic strategy. To this end, some studies have proposed a direct uptake of lipids from the interstitial fluid, given high levels of larger lipid particles and soluble nutrients can be found in this PDAC compartment. Hence, a the continuous stream of exogenous lipids including cholesterol, from blood to the interstitial fluid, is a major impediment for the intended anti-PDAC activity of dietary lipid restriction as well as inhibitors of de novo cholesterol biosynthesis such as statins. Yet, the lack of tangible targets and incomplete understanding of the delivery mechanism in tumor microenvironment has been the major obstacle for efficacious restriction of cancer cell access to the lipids borne in blood or interstitial fluid.

This study extends the prior analyses of human pancreatic adenocarcinoma and into a mouse genetic model of PDAC induced by pathogenic mutations in Kras and Trp53 in which we conditionally inactivated a distal cholesterol pathway gene Nsdhl (NAD (P) dependent 3-beta-hydroxysteroid dehydrogenase). Strikingly, cell lines derived from Nsdhl-deficient tumors rapidly died when cultured in lipid poor media, even though Nsdhl-deficient tumors grew very well in vivo, implying that these cancer cells were highly capable of extracting the exogenous cholesterol from the in vivo milieu. This led to investigating the mechanism of intratumoral delivery of lipids and the role of CAFs in the process.

In this study, it was discovered that CAFs transfer exogenous cholesterol and other nutrients via a cell contact-dependent transfer of plasma membrane fragments in a process known as trogocytosis (trogo=‘to gnaw’ or ‘to nibble’) whereby PDAC cells ‘nibble’ CAF membranes. Trogocytosis is the dominant means by which PDAC cells derive lipids from the tumor microenvironment and have outlined the mechanism by which such trogocytosis is regulated by a phospholipid scramblase anoctamin 6 (encoded by ANO6 gene). Since ANO6 is highly expressed in CAFs, ANO6 inhibition starves PDAC tumor cells of lipids.

Another typical feature of the pancreatic cancer is the high degree of immunosuppression, that has confounded the application of immunotherapies in this cancer type. This immunosuppressive feature of PDAC microenvironment has been linked to CAFs which can inhibit the activity of T cells in pancreatic tumors. Extending these studies, it was found that CAFs transfer their membrane cholesterol to cytotoxic T cells lymphocytes (CTLs) via trogocytosis causing their exhaustion, and inhibition of ANO6 in CAFs also attenuates this CTL immunosuppressive effects. Together, these findings led to proposing that blockade of ANO6 diminishes the CAF-mediated trogocytic transfer of exogenous cholesterol and may constitute a new effective therapeutic target against PDAC.

### 2. Results

### i. Pancreatic Cancer Stroma as a Transit Station for Lipid Delivery

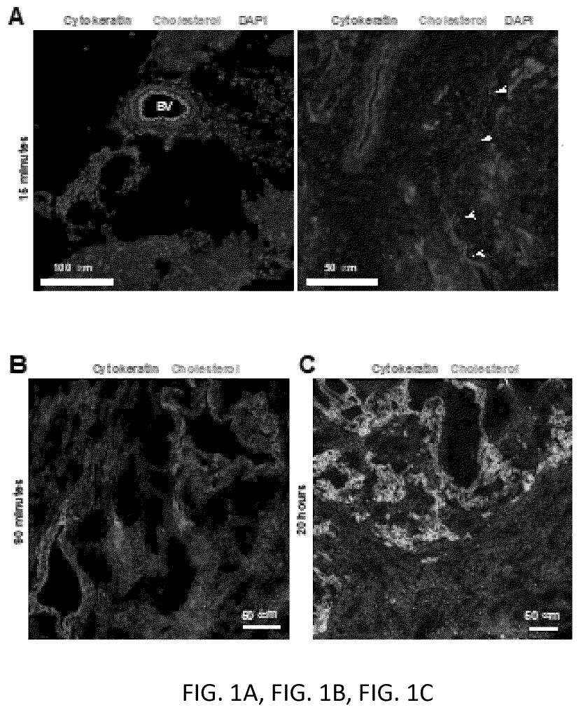

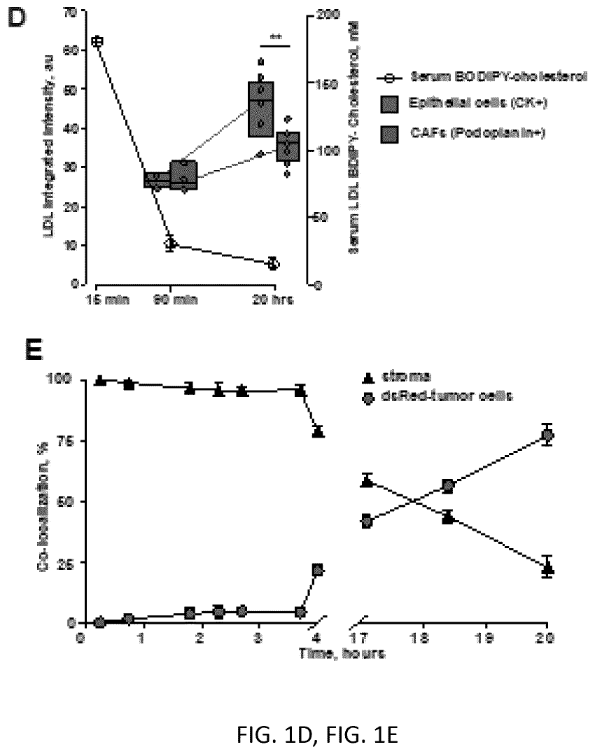

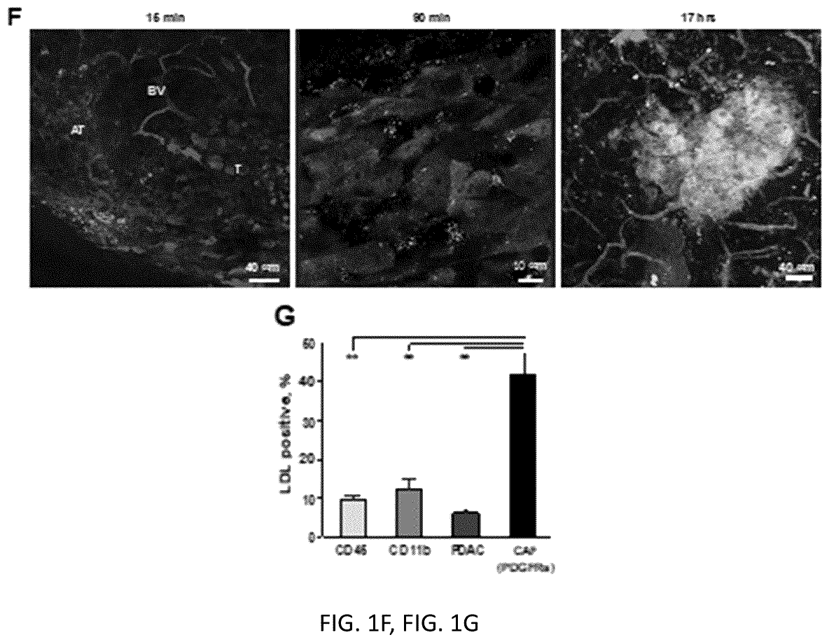

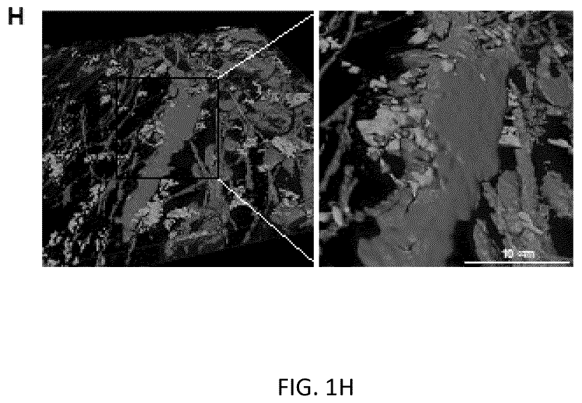

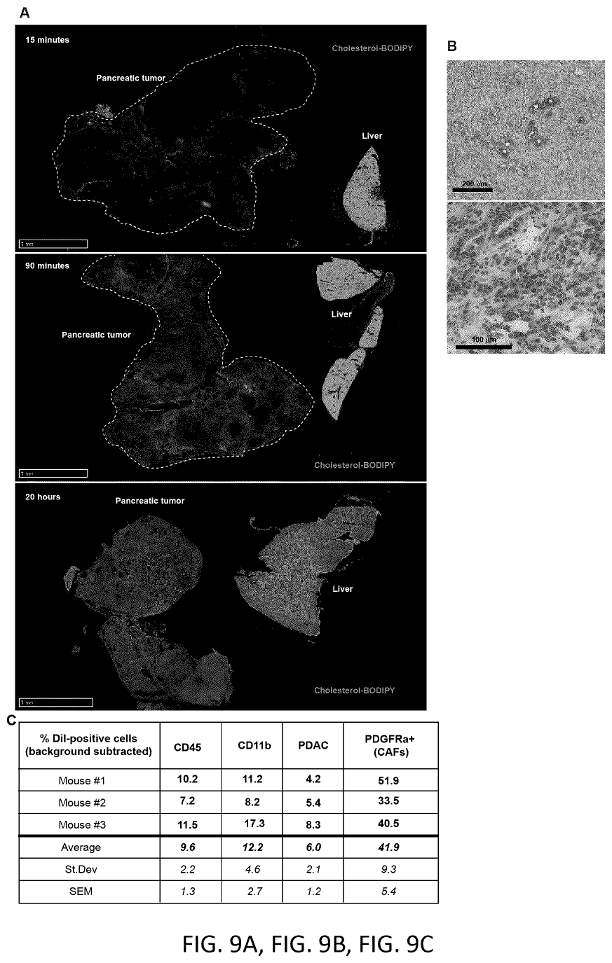

To gain insight into the mechanisms of uptake of exogenous lipids by PDAC cells in vivo, we determined the transit dynamics of blood-borne low-density lipoprotein (LDL) particles to cancer cells within the pancreatic tumor mass (FIG. 1). Using cytokeratins as a lineage biomarker of PDAC cells in cryosections of autochthonous KPC tumors (Pdx 1-Cre; KrasLSL-G12D; Trp53f/f), localization of intravenously injected LDL particles labeled with fluorescent BODIPY-cholesterol was traced. At fifteen minutes after injection, the fluorescent lipid signal was preferentially localized to blood vessels and intercellular spaces within the pancreatic tumors (FIG. 1A). The corresponding peak of serum BODIPY-cholesterol levels at 15 minutes rapidly declined by 90 minutes after injection due to rapid uptake of LDL particles by the tissues (FIG. 9A). Over time, from 90 minutes to 20 hours post injection, the fluorescent lipid signal predominantly localized to the cytokeratin-positive carcinoma cells (FIG. 1B-D, 9A,B). With the blood levels of BODIPY-cholesterol remaining low at later time points (FIG. 1D), it was concluded that such change in labeled cholesterol localization was caused by either intratumoral lipid redistribution, likely from CAFs to cancer cells, or by slower rate of LDL uptake by carcinoma cells compared to fibroblasts despite established upregulation of LDL receptors in PDAC carcinomas. To investigate further, syngeneic DsRed-labeled murine PDAC cells were implanted into the pancreas and the uptake of intravenously injected fluorescent LDL particles by cancer cells was traced (Fig. 1E, F) using multiphoton intravital microscopy. The 25 nm-sized LDL particles become visible by optical microscopy only when the fluorescent lipids enter the cells and aggregate in larger intracellular endosomes. A more extensive time course allowed for the comparison of the BODIPY-cholesterol signal between the DsRed-positive PDAC cells and the unlabeled stroma. Until four hours post-injection, the exogenously delivered lipids localized predominantly to the stroma, and not to the cancer cells (FIG. 1E, F). Following this initial delay, the fluorescently labeled LDL lipids progressively co-localized to the DsRed-positive pancreatic carcinoma cells at 17-20 hours post intravenous injection (FIG. 1E, F), whereas the amount of BODIPY-cholesterol in the stroma concurrently decreased to background levels. This observation was confirmed by flow cytometry analysis of single-cell suspensions which showed LDL retained at higher levels in PDGFRα-positive CAFs at 90 min post-injection. Contrastingly, only modest levels were gauged in fluorescently tagged PDAC cells, or in CD11b- or CD45-positive inflammatory myeloid cells (FIG. 1G). Most of the BODIPY-positive PDAC cells were in direct physical contact with BODIPY-positive stromal cells, with clearly visible cellular interdigitations (FIG. 1H). In sum, these results demonstrate that CAFs can be actively involved in the initial uptake of exogenous LDL lipids from the interstitial fluid, and that these exogenous lipids are being transferred to PDAC cells via a cell contact-dependent mechanism.

### ii. CAFs Provide Cholesterol to PDAC Cells Via Trogocytosis

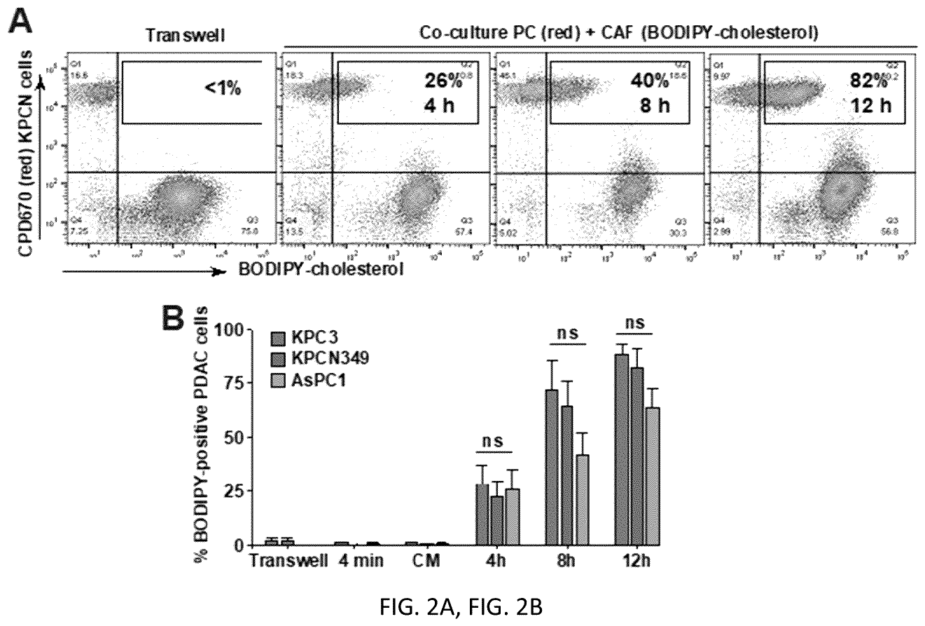

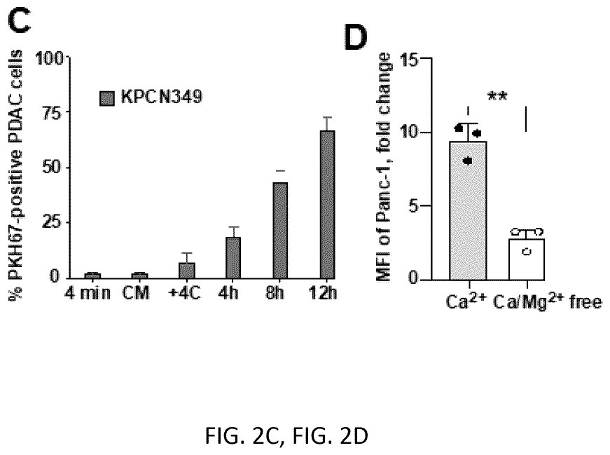

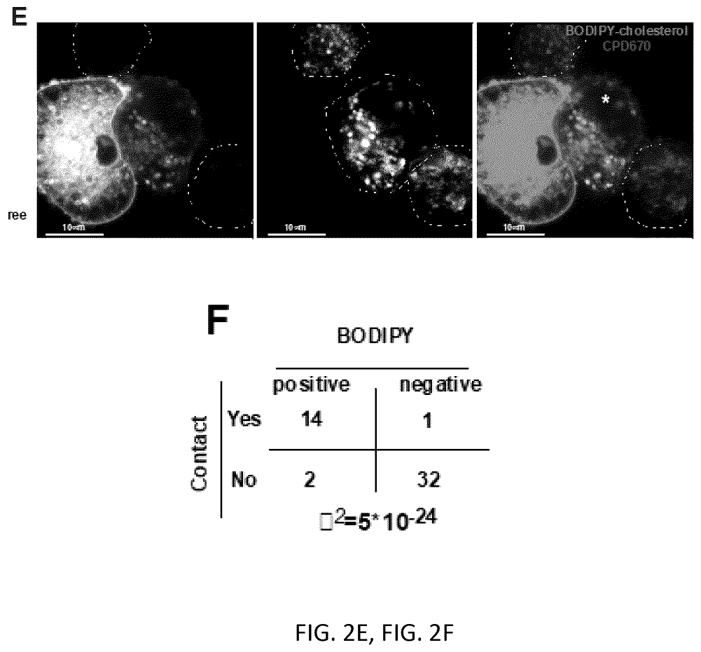

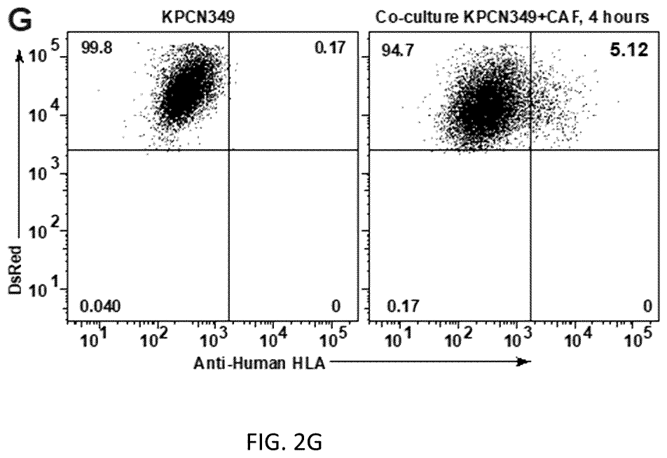

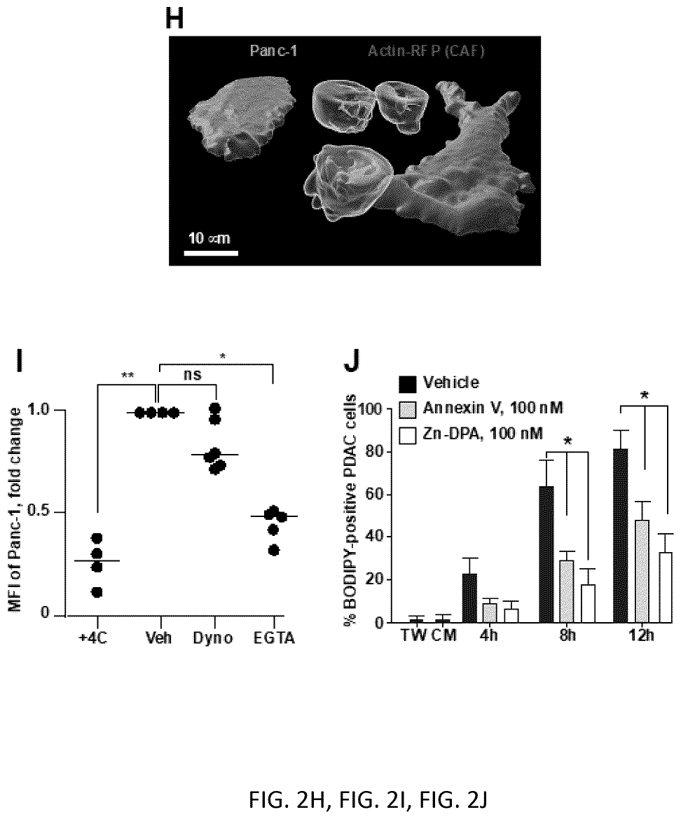

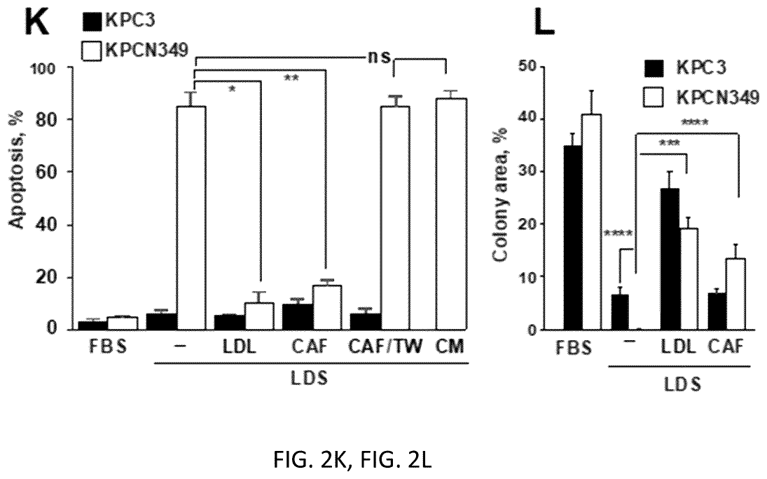

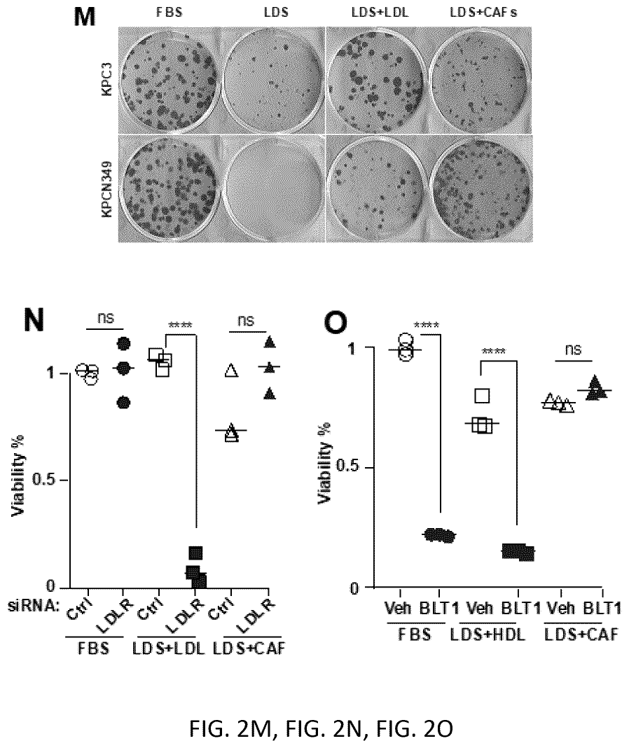

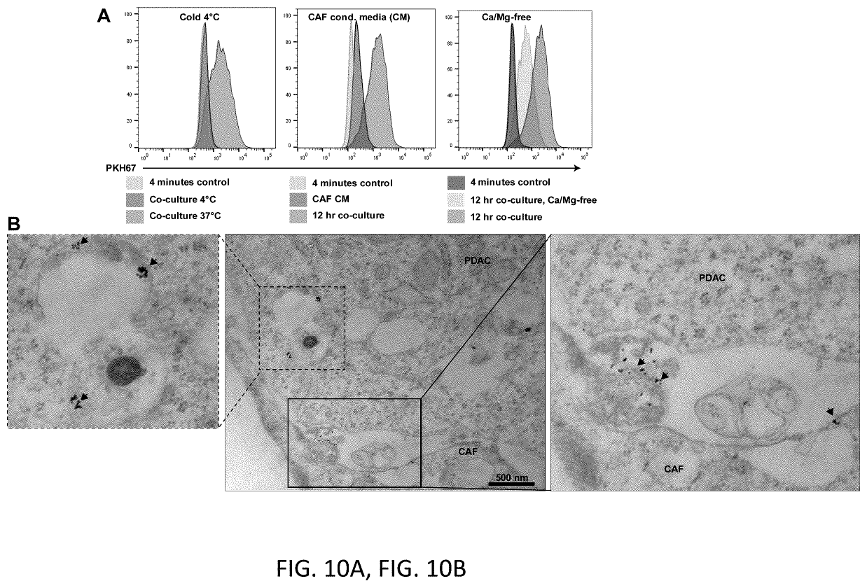

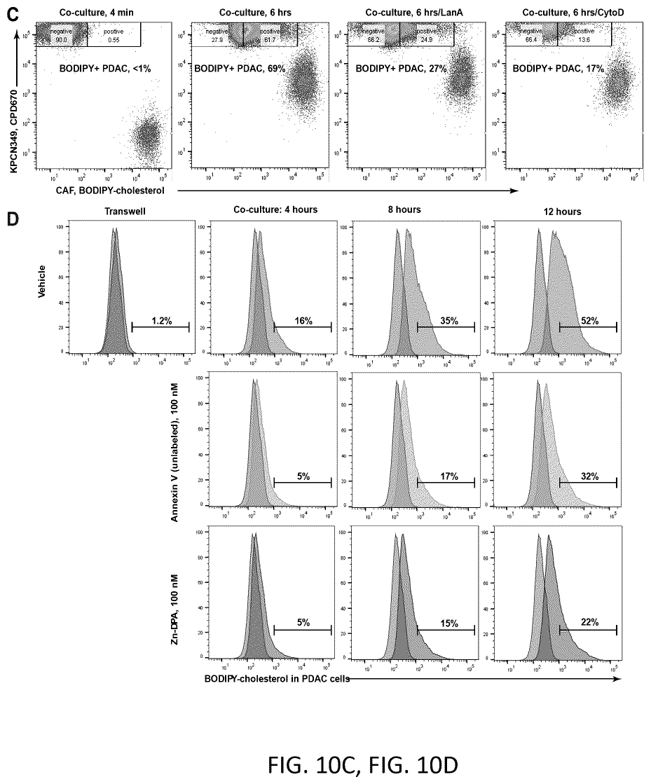

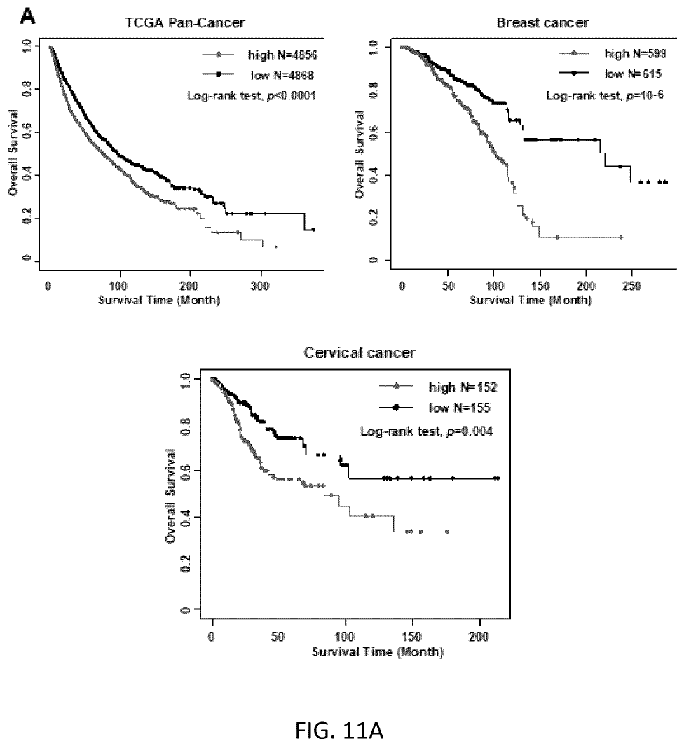

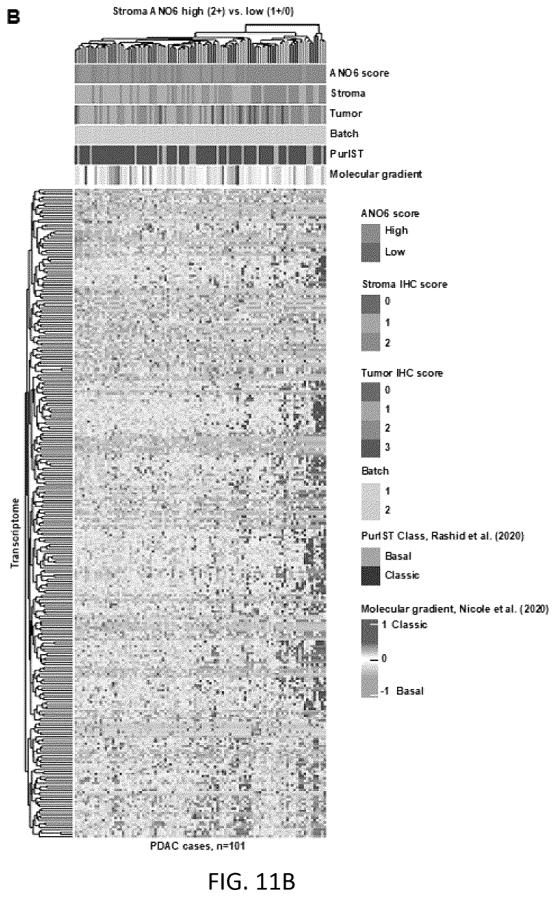

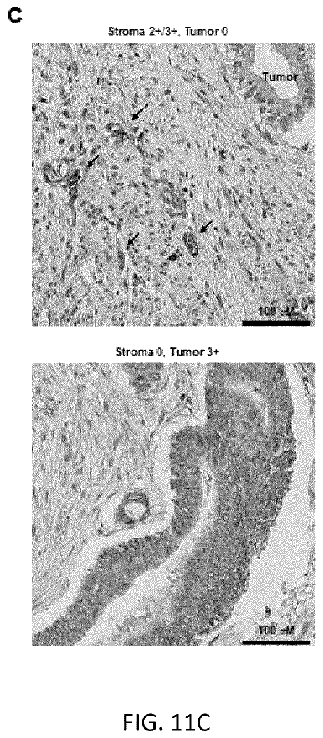

To model transfer of lipids in vitro, the membranes of pancreatic CAFs were fluorescently labeled with BODIPY-cholesterol and co-cultured CAFs with CPD670-tagged PDAC cells either in the same well, or in a transwell system where the cells were separated by a semi-permeable membrane preventing their physical contacts. Under contact-permissive conditions, PDAC cells rapidly acquired BODIPY fluorescence from CAFs as assessed by flow cytometry (FIG. 2A). All tested human (AsPC1, Panc-1) and murine (KPC3, KPCN349) PDAC cell lines rapidly (4-12 hours) readily acquired CAF membranes (FIG. 2B) labeled with BODIPY-cholesterol, or lipophilic dye PKH67 (FIG. 2C, 10A). The cell contact dependency of such transfer of membranes from CAFs to PDAC cells was confirmed by observing minimal acquisition of the CAF membrane label by PDAC cells: (i) in transwell co-cultures; (ii) from 3-day CAF conditioned media (CM), or (iii) in co-cultures at +4° C. (FIG. 2B, C, 10A). Importantly, CAF membrane transfer was markedly suppressed in calcium and magnesium-free media (FIG. 2D, 10A). Microscopic imaging of PDAC cells in co-cultures with CAF demonstrated formation of sustained, synapse-like contacts between CAF and PDAC cells in which CAF PM protrusions are acquired by contacting PDAC cells, but not by the bystander cancer cells not engaged in direct physical contact with CAFs (FIG. 2E, F). This mechanism of membrane uptake has been described as trogocytosis by which the receiver cell engages in a direct physical contact to acquire protrusions of the donor cell's PM which was evident in electron microscopy imaging of colloidal gold-labeled CAFs co-cultured with carcinoma cells (FIG. 10B). Trogocytosis has been previously demonstrated for both immune cell synapses, and in human development. In CTLs, trogocytosis causes uptake of the human leukocyte antigen (HLA) MHC-I and peptide complexes from antigen-presenting cells. To confirm the CAF PM uptake by PDAC cells represents trogocytosis, co-culture of human CAFs with mouse PDAC cells resulted in acquisition and re-expression of human HLA proteins on the surface of murine PDAC cells (FIG. 2G). Furthermore, trogocytosis can be partially blocked by inhibitors of the cytoskeleton as was the case with the actin polymerization inhibitors Latrunculin B and Cytochalasin D, which suppressed uptake of CAF PMs by PDAC cells (FIG. 10C). Introduction of red fluorescent protein (RFP)-tagged actin to CAFs revealed transfer of membrane protrusions containing actin to PDAC cells (FIG. 2H). Furthermore, the dynamin inhibitor dynasore did not affect the uptake of membrane lipids from human CAFs by Panc-1 cells in co-cultures (FIG. 21) indicating the uptake mechanism is unlikely to be regulated via a receptor-mediated endocytosis. As phosphatidylserine (PtdSer) exposure could be a potential “eat me” signal for uptake of cellular membranes, CAF and PDAC cells were co-cultured in the presence of PtdSer binding recombinant annexin V, or zinc-dipicolylamine (Zn-DPA), revealing significantly reduced uptake of BODIPY-labeled CAF membranes (FIG. 2J, 10D). In sum, the CAF-to-PDAC membrane lipids transfer is trogocytosis-dependent and is regulated in a manner dependent on extracellular calcium and PtdSer exposure.

To further investigate the functional significance of CAF membrane trogocytosis, we examined the ability of CAFs to support the viability of cholesterol auxotrophic PDAC cells in vitro. In the absence of lipids and cholesterol in culture media (lipid-depleted serum, or LDS, 72 hours), murine KrasmutTrp53−/− (KPC) cell lines with an intact Nsdhl gene did not undergo apoptosis (closed columns, FIG. 2K). In contrast, Nsdhl-null KPCN349 cells underwent apoptosis (open columns, FIG. 2K) which could be rescued by either direct addition of 50 μM cholesterol in LDL particles, or by co-culture with human CAFs derived from PDAC patients. This rescue from apoptosis in LDS was not conferred by CAF-conditioned medium (CM), or in CAF-PDAC co-cultures in transwell plates (TW, FIG. 2K). Further, CAFs maintained the growth of PDAC cells in long-term clonogenic cultures in the absence of exogenous lipids (LDS) to the extent comparable to LDS supplemented with 50 μM cholesterol in LDL, or FBS (FIG. 2L, M). CAF co-cultures fully rescued the viability of PDAC cells regardless of LDL receptor inactivation with siRNA (FIG. 2N), or blockade of HDL receptors with BLTI (FIG. 20) indicating CAFs are necessary and sufficient for the viability and growth of cancer cells via trogocytosis.

### iii. ANO6 Scramblase Confers Poor Survival in Pancreatic Cancer

To gain mechanistic insight into regulation of trogocytosis in PDAC, candidate enzymes responsible for PtdSer externalization in CAFs were sought out. Exposure of PtdSer on the outer leaflet of PM is the consequence of lipid redistribution, or “scrambling”, between the inner and outer leaflets of the PM to be regulated by lipid scramblases. In contrast to Xk-related 8 (XKR8) scramblase promoting PtdSer externalization on the PM during apoptosis, phospholipid scramblase anoctamin 6 (ANO6) induces PtdSer externalization in response to elevated intracellular calcium under physiologic stimuli in non-apoptotic cells. Notably, trogocytosis of CAF membranes by Panc-1 cells was markedly reduced in calcium-free media, or at +4° C. (FIG. 2D, I, 10A). Taken together, these results suggest that CAF trogocytosis may involve a calcium-dependent PtdSer scramblase of the anoctamin family.

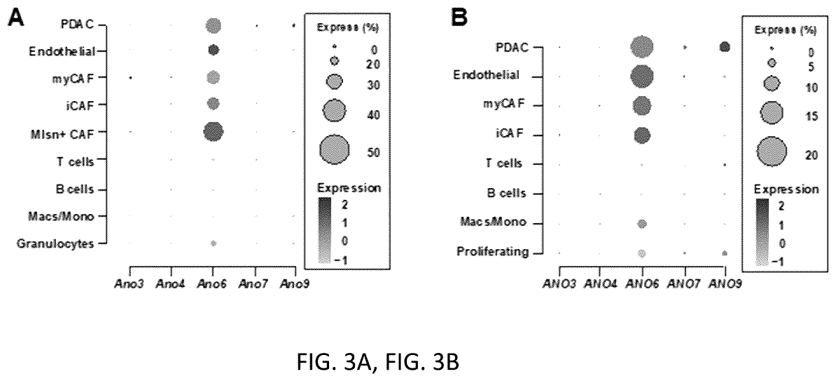

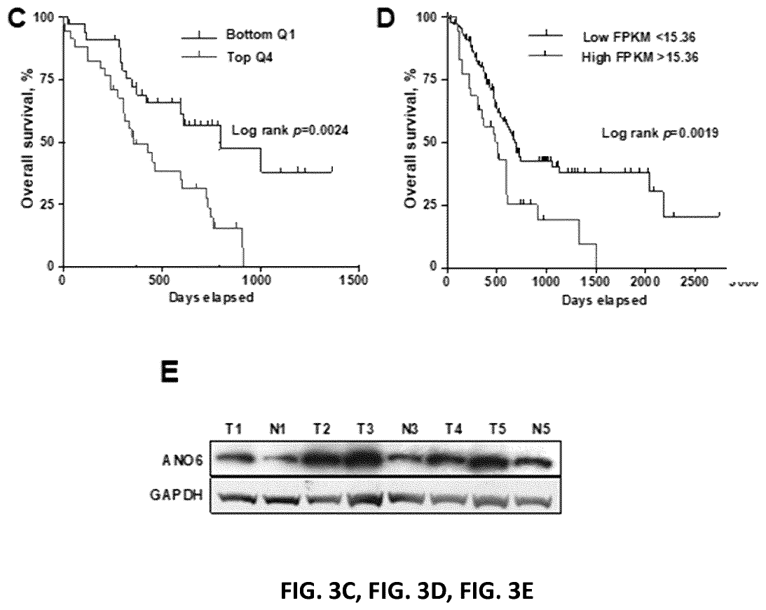

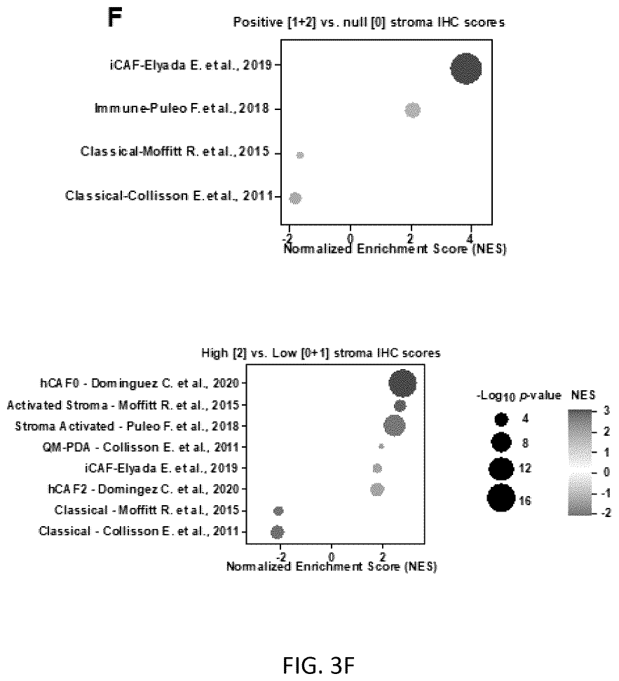

Since ANO-family scramblases have not been previously implicated in cancer, their expression in mouse and in human pancreatic cancer were examined using single-cell RNA sequencing datasets (FIG. 3). Of the five ANO-family paralogs in mice (Ano3, 4, 6, 7 and 9) with experimentally confirmed PtdSer scramblase activity, only Ano6 was expressed in CAFs, endothelial cells and adenocarcinoma cells in defined pancreatic tumor cell populations (FIG. 3A, B). Further, high expression of ANO6 protein (FIG. 3C) or transcript (FIG. 3D) are associated with worse overall survival of patients with stages 1-3 of PDAC as well as in TCGA breast and cervical cancers (FIG. S3A). ANO6 was highly expressed in a panel of fibroblastic cell lines obtained from surgical pancreatic adenocarcinoma tissues and in fibroblastic cells harvested from adjacent, non-malignant, human pancreatic tissues (FIG. 3E). The effects of ANO6 expression were next investigated in the specific cellular compartments of PDAC tumors using immunohistochemistry labeling of ANO6 on a panel of 101 surgical pancreatic adenocarcinoma samples for which bulk RNA sequencing data were available (FIG. 3F, 11B, 11C). Suggestively, higher ANO6 stromal staining was associated with carcinoma poor carcinoma differentiation status (p=0.01, Kruskal-Wallis rank sum test), and signatures of inflammatory CAFs and activated stroma (FIG. 3F). Conversely, low or negative expression of ANO6 in stromal fibroblasts was associated with classical subtype of PDAC. Together, these results support a tumor-promoting role for CAF-expressed ANO6 in pancreatic adenocarcinoma, and potentially other human cancers.

### iv. ANO6 Regulates Tumor-Promoting Function of CAFs

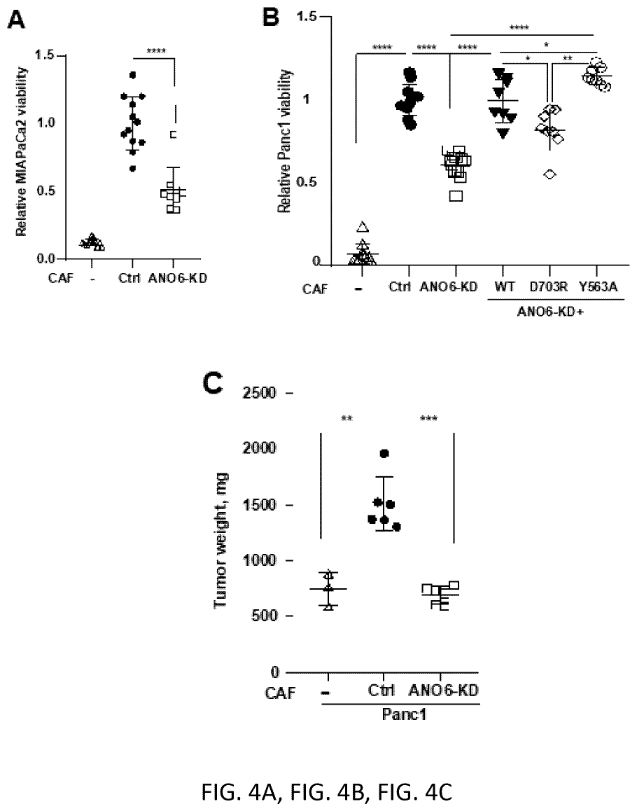

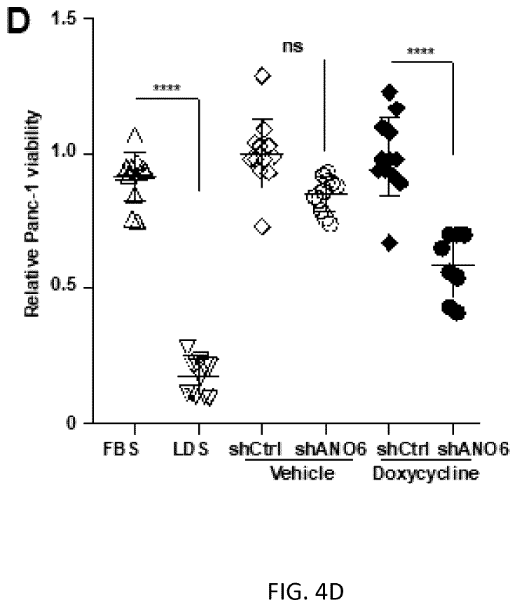

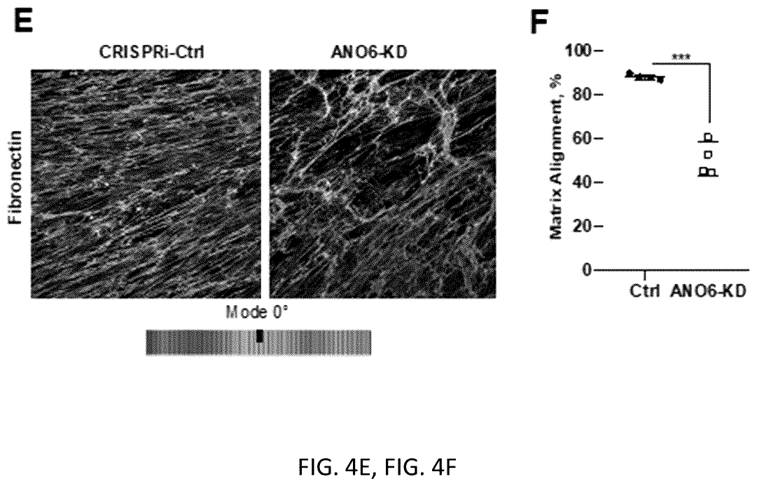

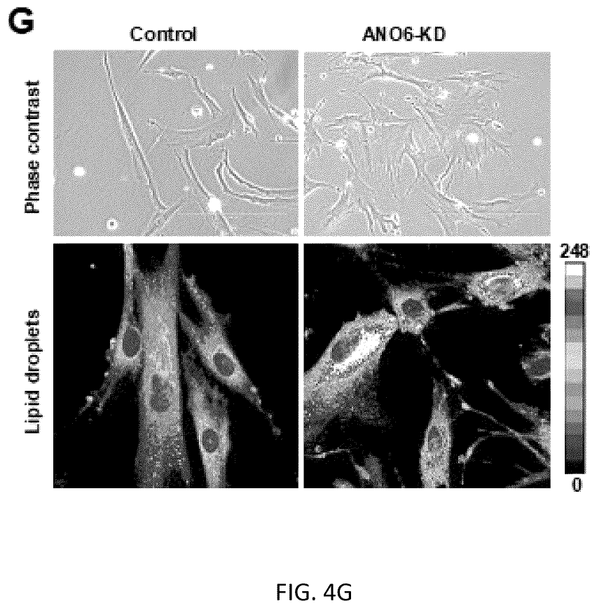

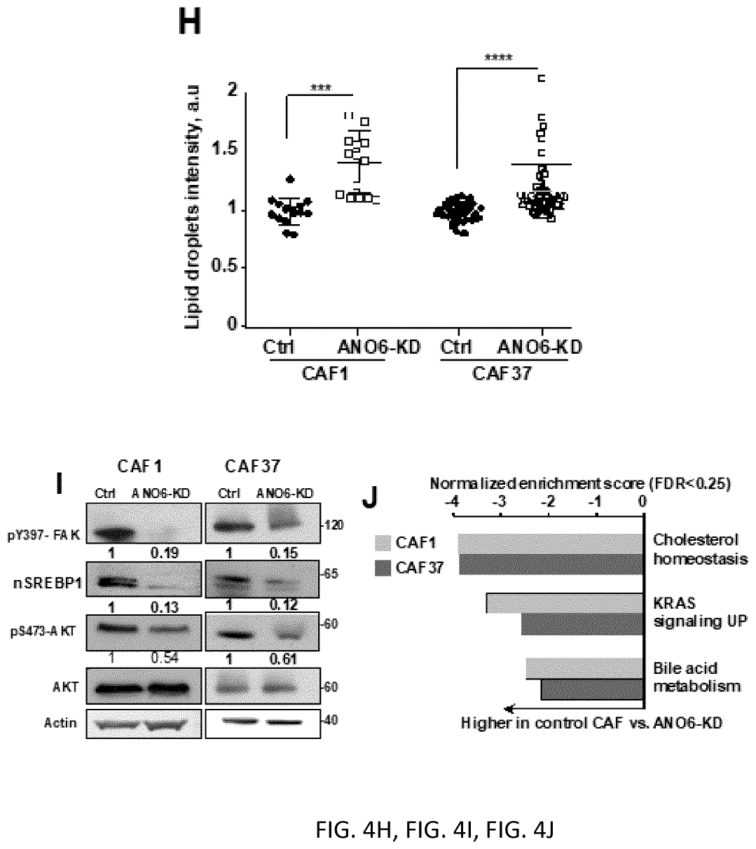

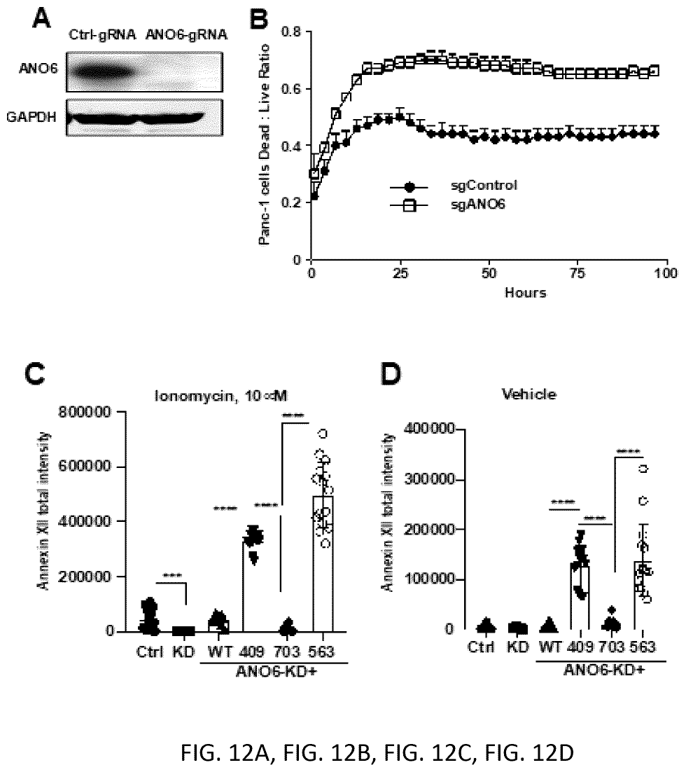

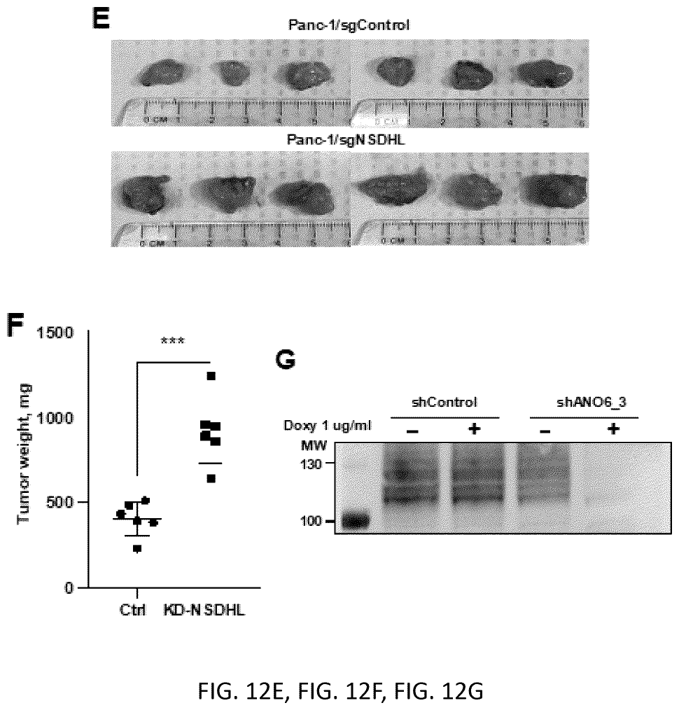

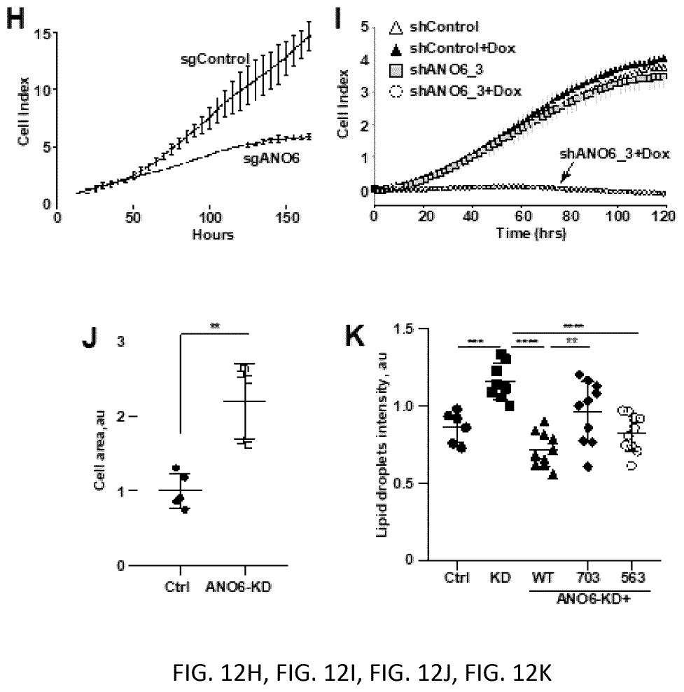

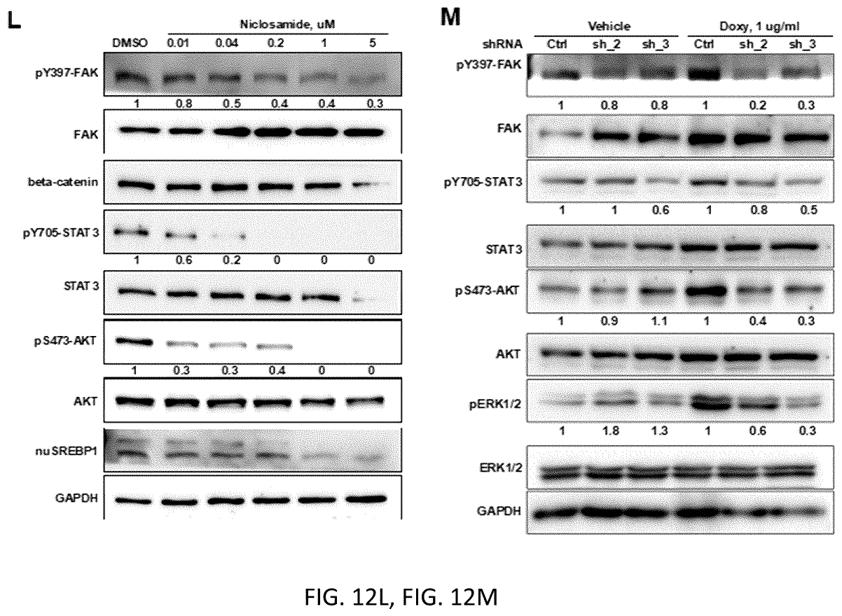

To examine the role of ANO6 in the tumor-promoting function of CAFs, we depleted ANO6 in CAFs using CRISPRi (FIG. 12A). We determined that ANO6 inactivation in CAFs is critical for the ability of these cells to provide cholesterol and thus support the viability of the cholesterol auxotroph NSDHL-depleted MIAPaCa-2ΔNSDHL cancer cells in co-cultures under lipid poor media (FIG. 4A) as compared to control CAFs modified with non-targeting gRNAs (i.e., ANO6-positive CAFs). Nearly identical results were obtained upon ANO6 inactivation in CAFs in co-cultures with Panc-1ΔNSDHL PDAC cells (FIG. 4B, 12B). Conversely, cancer cells viability was rescued by expression in CAFs of the wild-type ANO6, or of a constitutively active Y563A ANO6 mutant (FIG. 4B) whereas co-cultures of Panc-1ΔNSDHL cells with CAFs expressing the scramblase-inactive ANO6 D703R variant which interferes with the calcium-binding pocket of the protein (FIG. 12C, D) resulted in lower viability compared to the wild type ANO6 (FIG. 4B). ANO6-proficient CAFs doubled the growth of Panc-1ΔNSDHL orthotopic cholesterol-auxotroph tumors (FIG. 4C, 12E, F) whereas ANO6-null CAFs failed to promote the growth of the xenografts (FIG. 4C). In an orthogonal approach, transient depletion of ANO6 in human CAF lines using doxycycline-inducible shRNA (FIG. 12G) resulted in reduced Panc-1ΔNSDHL viability in co-cultures supplemented with lipid-poor scrum (LDS, FIG. 4D) as compared to CAFs modified with a non-targeting shControl. Deficiency of ANO6 resulted in conversion of CAFs to quiescent naïve fibroblast-like phenotype as assessed by the reduced growth in vitro (FIG. 12H,I), and loss of anisotropic extracellular matrix characterized by parallel alignment of fibronectin fibers (FIG. 4E,F). Furthermore, ANO6-KD fibroblasts acquired stellate morphology (FIG. 4G, 12J), and accumulated lipid droplets (FIG. 4G, H) in an ANO6-dependent manner (FIG. 12K). Exogenous re-expression of ANO6, but not of the inactive D703R ANO6 mutant, reduced lipid droplets in ANO6-KD CAFs (FIG. 12K).

In contrast to naturally tumor-suppressive naïve fibroblastic cells, pro-tumoral CAF are activated in a reciprocal manner by their self-generated extracellular matrix and typically express matrix-activated integrin signaling as demonstrated by phosphorylated focal adhesion kinase (FAK) and AKT. Of note, ANO6-KD CAFs exhibited markedly reduced levels of autophosphorylated focal adhesion kinase (pY397) FAK, and (pS473) AKT (FIG. 4I, 12L, M), suggesting that ANO6 inhibition indeed triggers a functional CAF “normalization” and proliferative quiescence. Accordingly, transcriptomes of ANO6-depleted fibroblasts showed reduced expression of proliferative Hallmark “KRAS signaling UP” and “cholesterol metabolism” signature genes (FIG. 4J) consistent with reduced nuclear SREBP1 (FIG. 4I) as compared to control ANO6-proficient CAFs. Taken together, these results support the idea that ANO6 plays a critical role in tumor-promoting function of CAFs.

### v. PDAC-CAF Synapses Activate Orail-STIM Calcium-Dependent Signaling

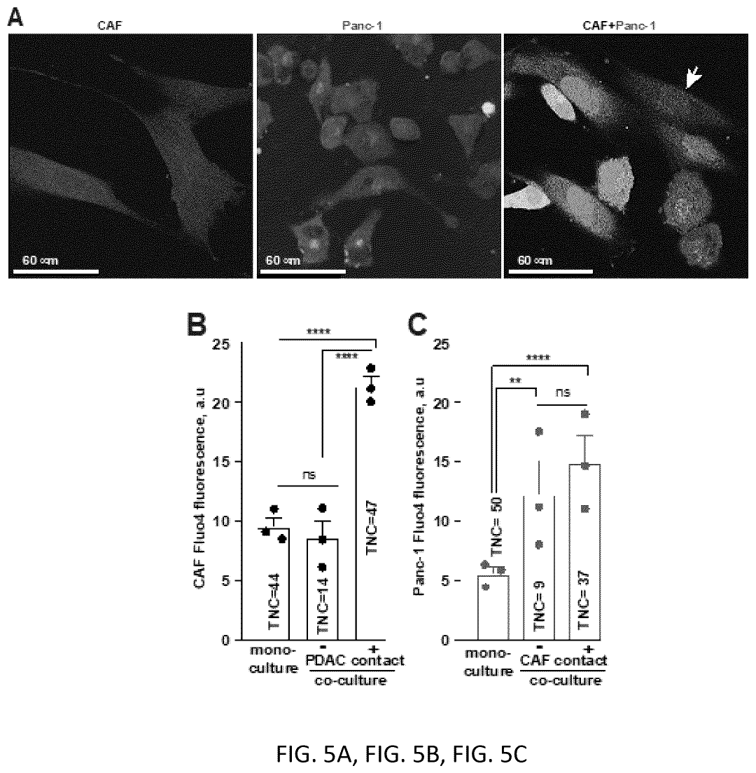

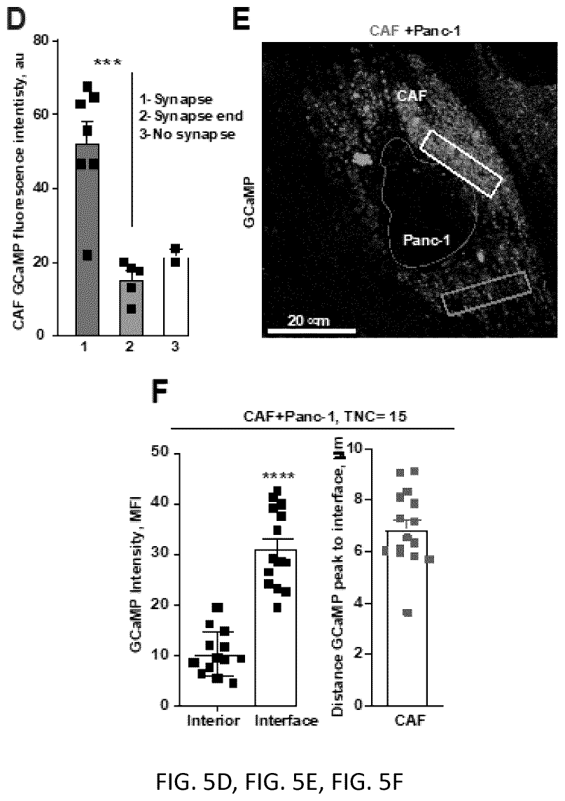

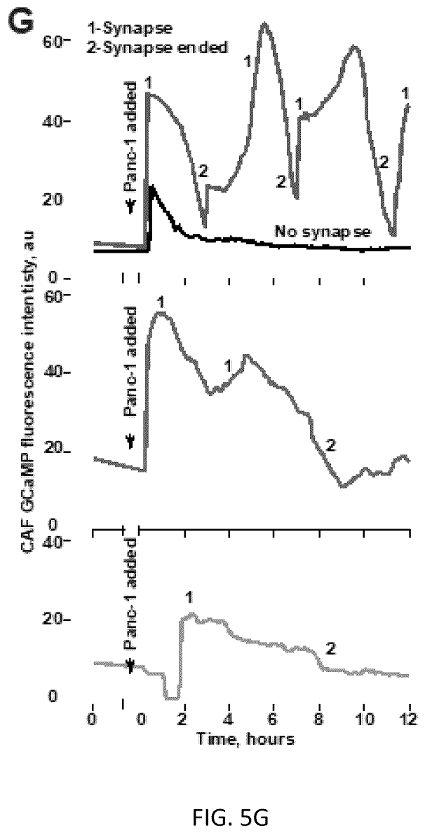

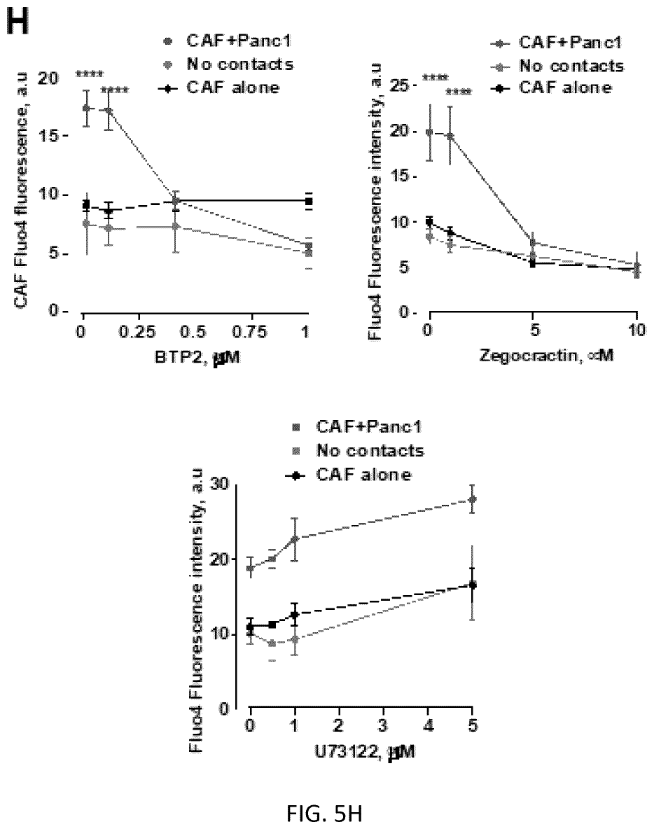

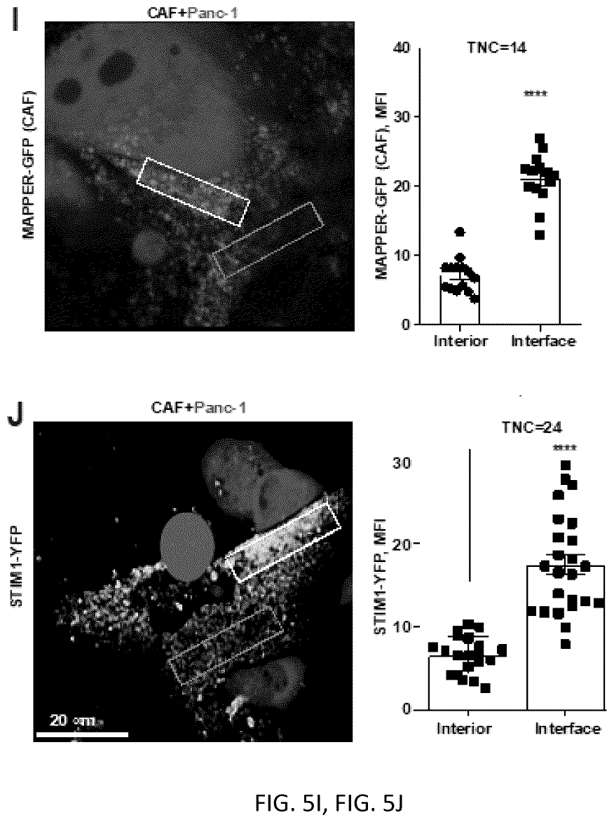

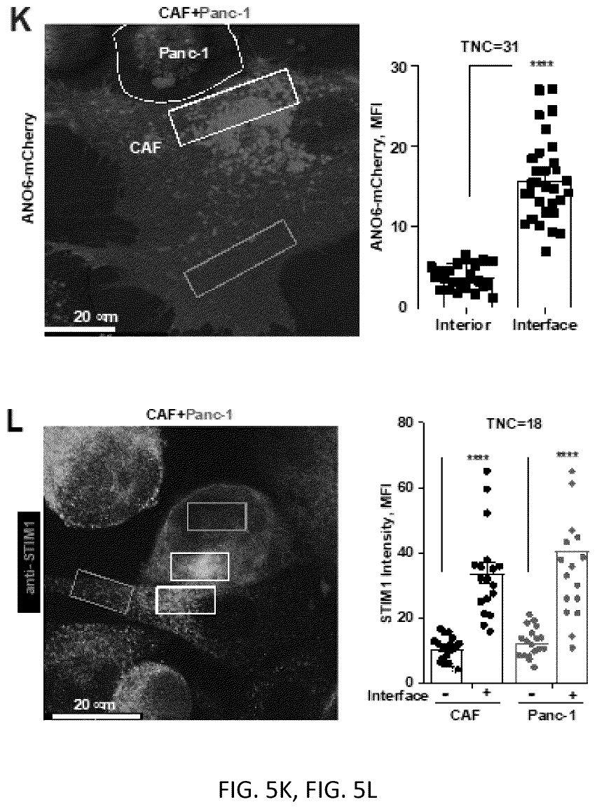

Based on the results that CAF membrane trogocytosis is induced at CAF-cancer cell membrane contacts in a manner dependent on calcium entry in the cell (FIG. 2D-I), it was next sought to determine the regulation of extracellular calcium entry in CAFs. Using CAFs labeled with the fluorescent intracellular calcium sensor Fluo4, it was determined that CAFs engaged in membrane contacts with cancer cells exhibited elevated calcium-dependent fluorescence relative to either CAFs not in contact with cancer cells was or to CAF monocultures (FIG. 5A, B). interestingly, cytosolic calcium contentin Panc1 cells was also elevated in co-cultures with CAFs, suggesting that their may be reciprocal engagement of calcium signaling during CAF-PDAC engagement (FIG. 5A, C). To track Ca2+ signaling in CAFs over longer time intervals in co-cultures with Panc-1 cells, we transfected CAFs with a well established genetically-encoded cytoplasmic calcium reporter construct GCaMP. The GCaMP signal was observed exclusively in CAFs engaged in sustained contact with cancer cells in a contact-polarized manner (FIG. 5D-F). The GCaMP fluorescence rapidly terminated when the CAF-PDAC synapse-like contacts ended (FIG. 5G).

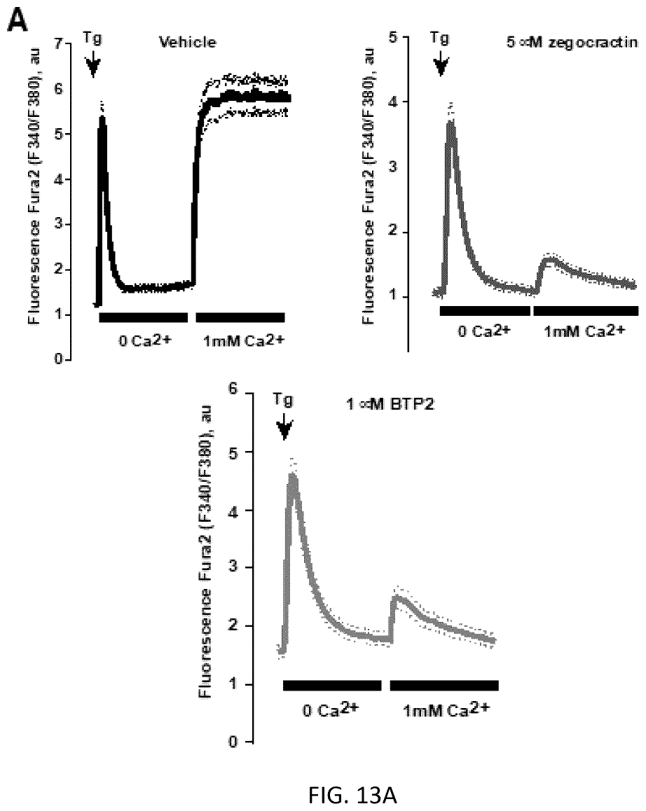

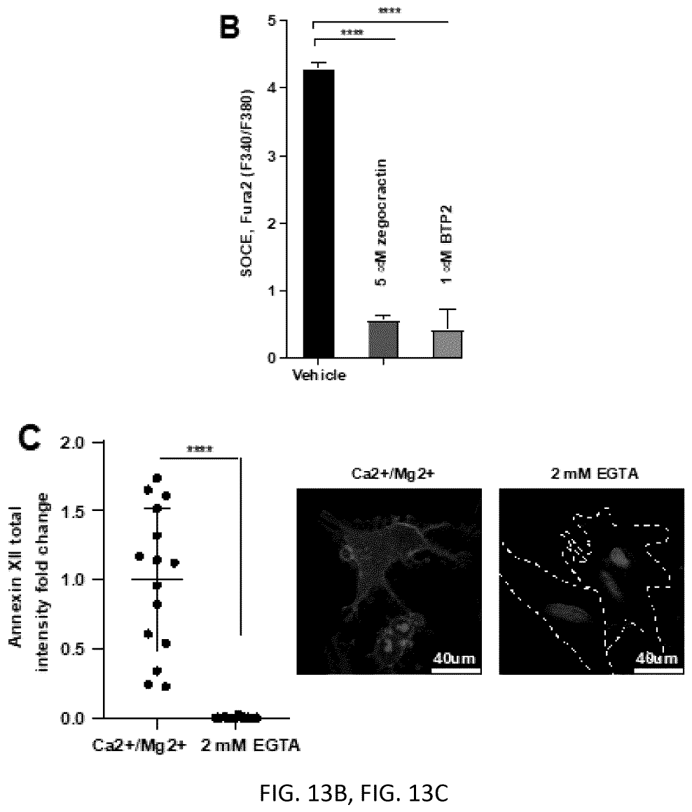

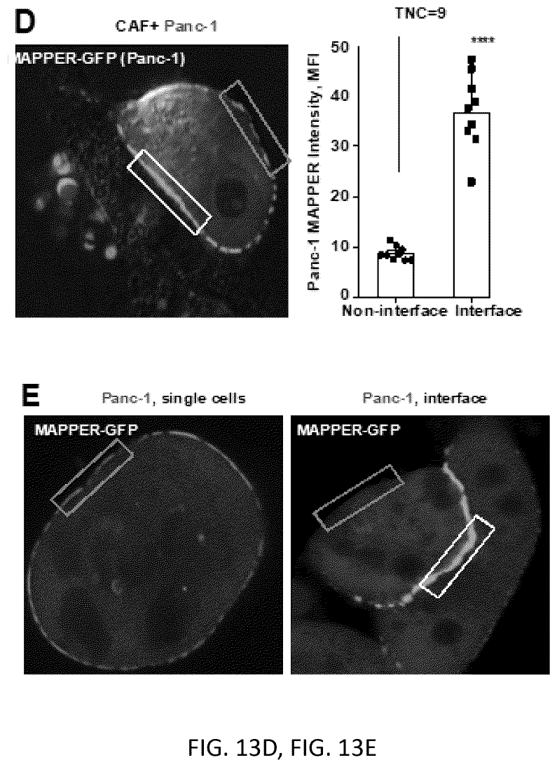

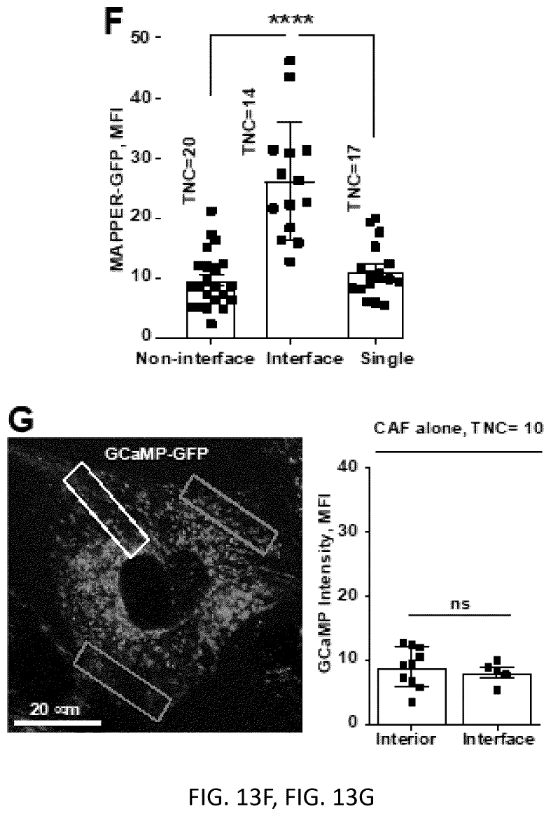

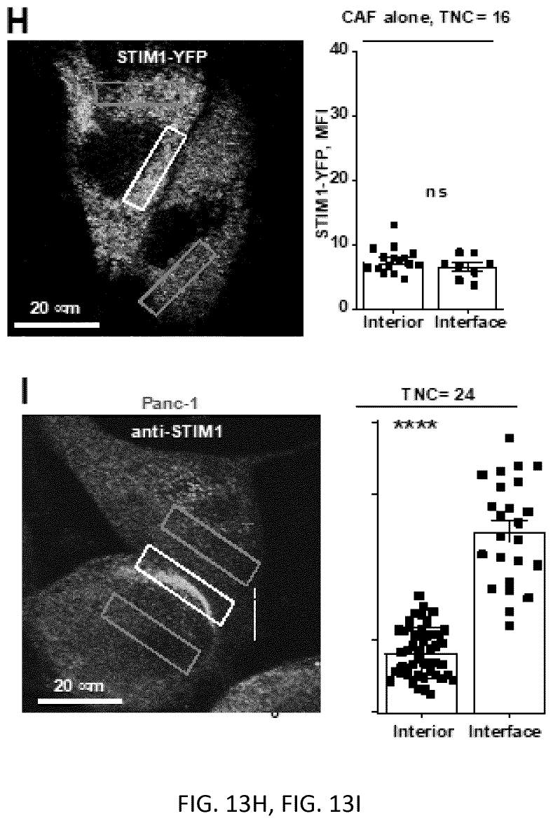

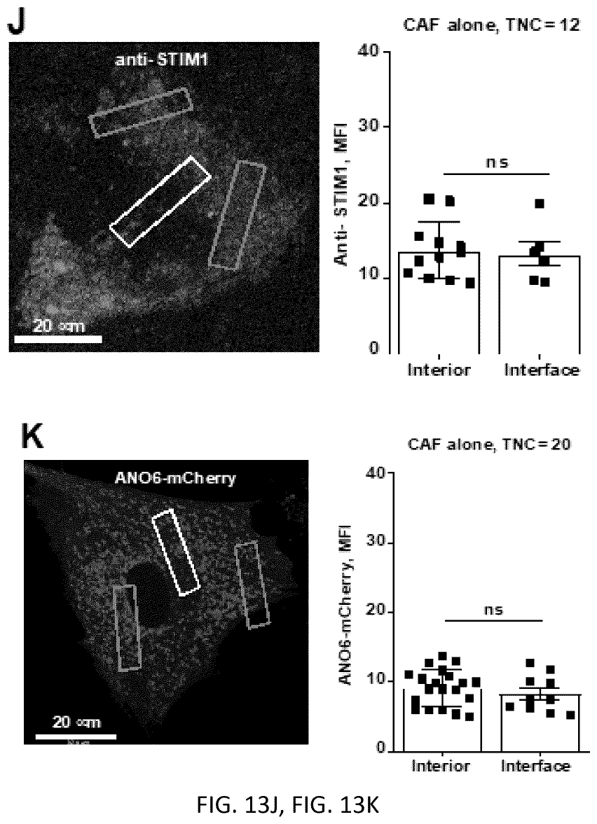

To identify the physiological calcium channels activating ANO6 in CAF cytosol following interaction with PDAC cells, we used a pharmacological strategy to probe distinct routes of Ca2+ entry into the cytosol. Two selective store-operated calcium entry (SOCE) inhibitors BTP2 and zegocratin (FIG. 5H, 13A, B) effectively blocked cytosolic Ca2+ elevation in CAFs engaged in heterotypic cell-cell contacts with PDAC cells. Notably, PtdSer externalization was blocked by extracellular calcium chelator EGTA (FIG. 13C). Orai channels are known as the pore-forming subunit mediating SOCE. To better understand how Orai might be activated, we tested the phospholipase C inhibitor U73122, finding no effect on Fluo4 signal (FIG. 5H). This observation both reinforces our conclusion that the calcium enters through a PM calcium channel and eliminates any possibility that ER calcium release could account for the observed cytosolic calcium elevation.

Based on these findings, the possibility that heterotypic CAF-cancer cell membrane contacts could functionally simulate immunological synapses (IS) was considered, in which the Ca2+ signaling machinery becomes polarized at sites of cell-cell membrane contacts. In addition to Orai, members of the Stromal Interacting Molecule (STIM) family of ER Ca2+ sensors are required components of SOCE; STIMs are the endogenous activators of Orai channels via physical association. STIM1 and STIM2 respond to ER Ca2+ depletion by translocating within the ER towards ER-PM junctions (typically in ˜1 minute). However, the IS stabilizes (˜2 h), both STIM and Orai translocate towards the IS via not fully established mechanisms. To assess the possibility of a similar occurrence at heterotypic PDAC-CAF interaction sites, ER-PM junctions of CAFs were marked using GFP-MAPPER, an artificial construct that labels sites of close ER-PM apposition. Based on prior reports, STIM1 should be detected at these ER-PM junctions. Strikingly, CAF GFP-MAPPER puncta were polarized in the direction of heterotypic CAF-PDAC cell-cell junctions in the co-cultures (FIG. 5I, 13E-G), yet these were distributed throughout the entire cell contour in CAF monocultures (FIG. 13H-K). Further, since ANO6 activity is dependent on STIM/Orai, and since STIM is known to modulate the function of numerous PM resident proteins, it was reasoned these proteins may physically interact at ER-PM contact sites. In support of this concept, transfection of fluorescently labeled STIM1 and ANO6 showed co-enrichment of these proteins at heterotypic PDAC-CAF contact sites (FIG. 5J, K), in contrast to both proteins being evenly distributed in CAF monocultures (FIG. 13H-K). A similar localization was observed in cancer cells and CAFs engaged in synapse-like contacts as assessed by a direct labeling of endogenous STIM1 (FIG. 5I). These findings indicate that the functions of STIM, Orai and ANO6 in CAFs are closely coupled, and ultimately, are required for PtdSer externalization to facilitate trogocytosis of CAF PM by recipient cells (e.g., PDAC cells).

### vi. A Hydrophobic Pocket of ANO6 is Critical for Lipids Delivery In Vivo

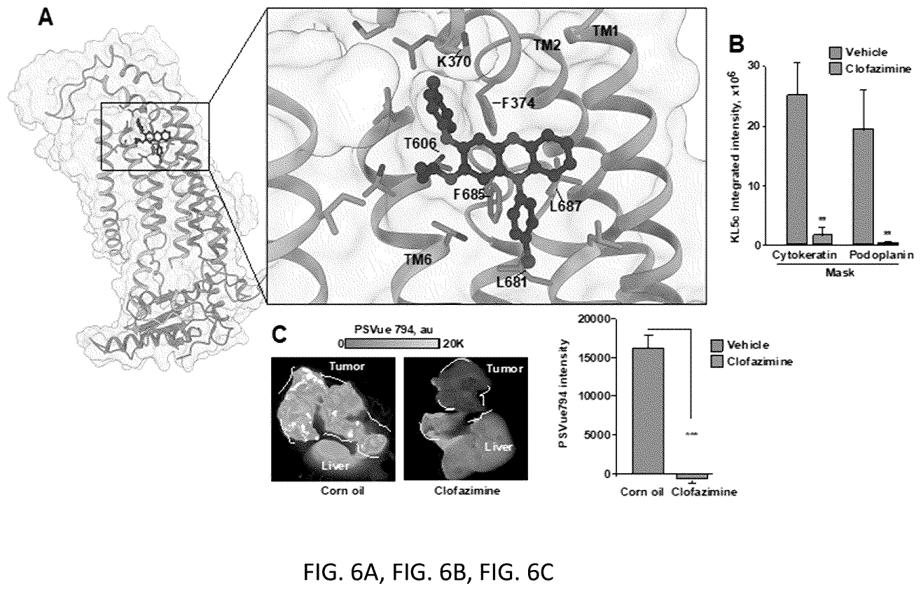

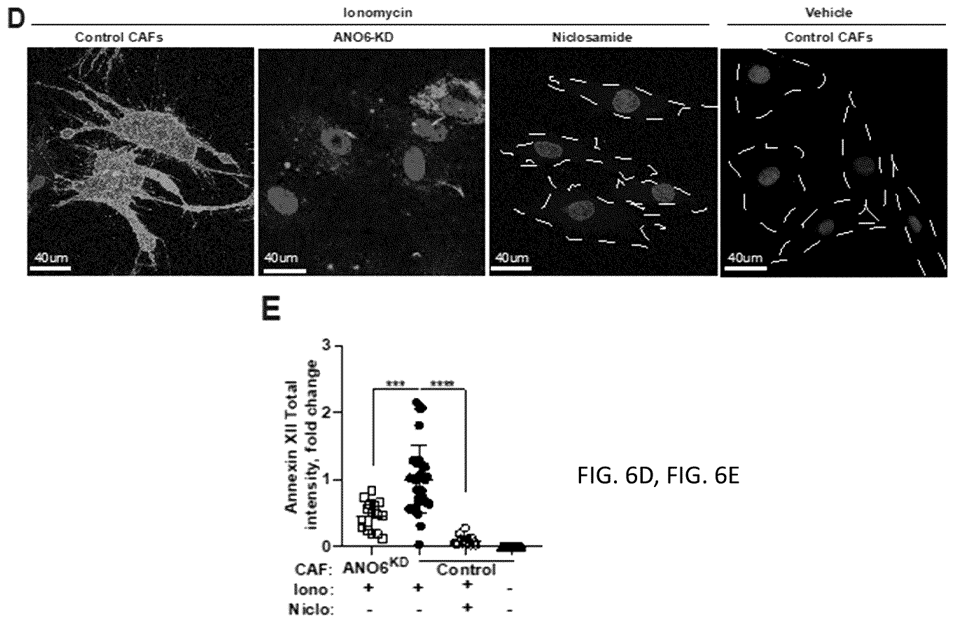

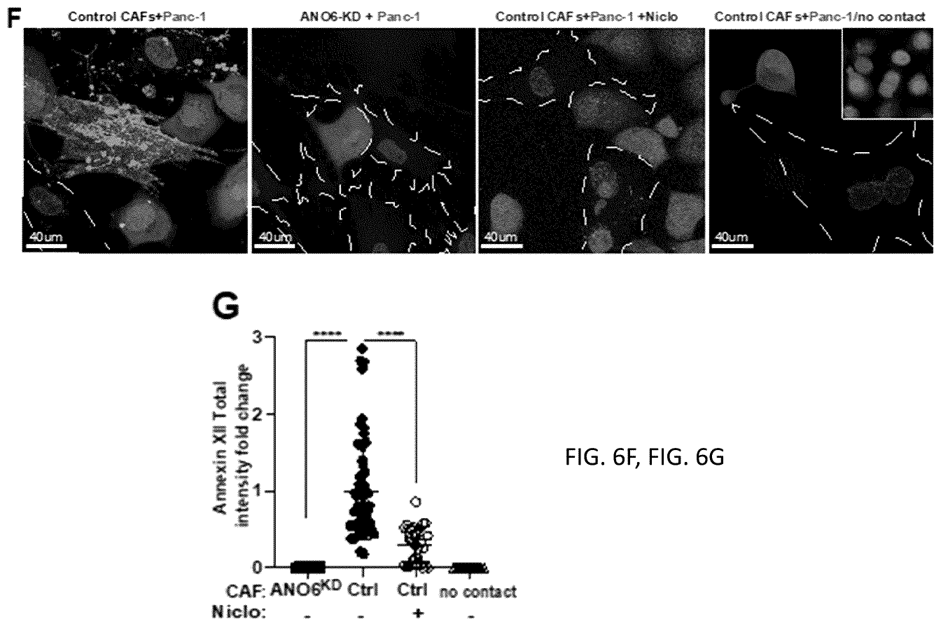

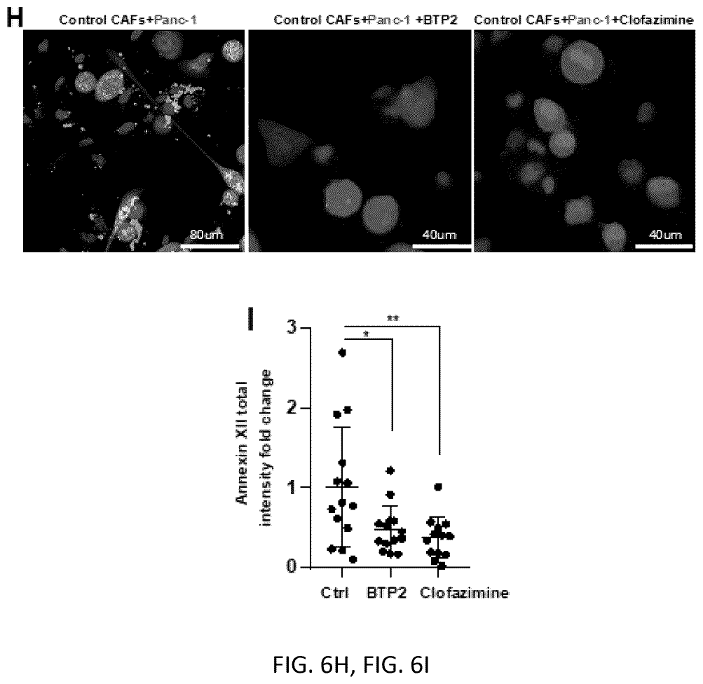

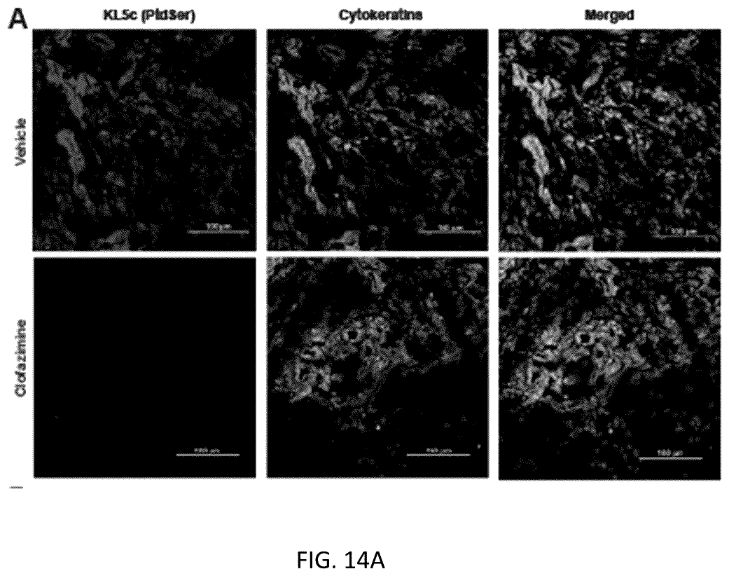

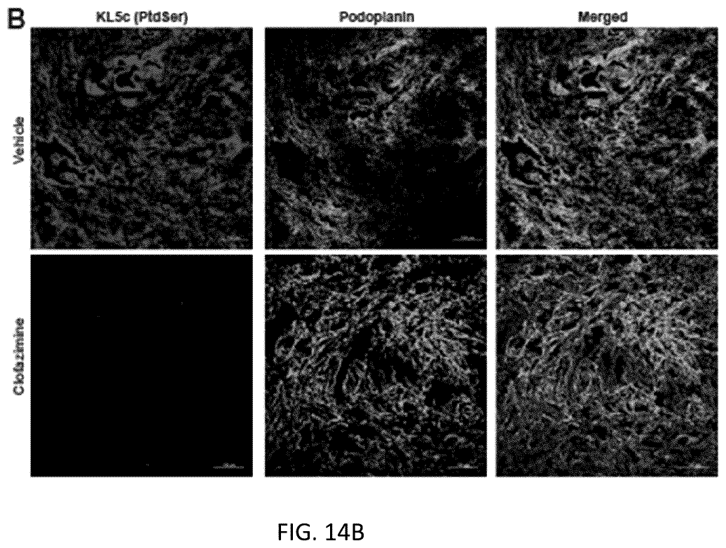

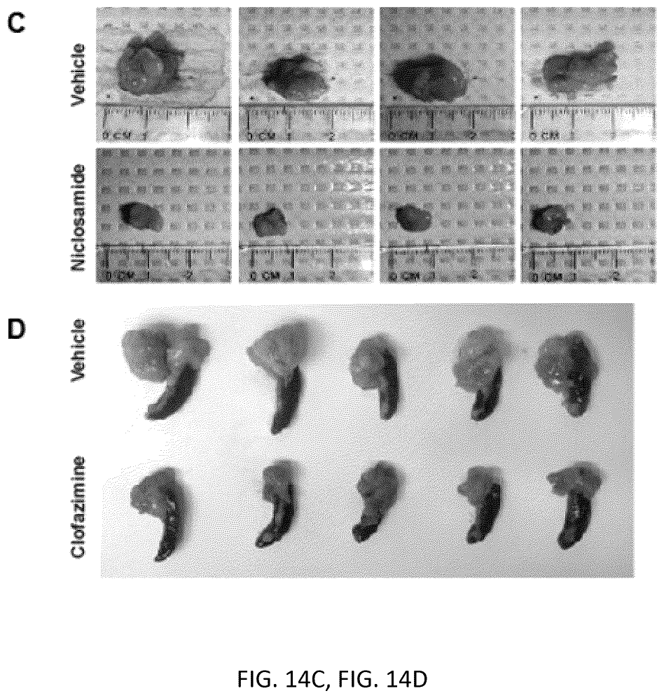

It was next examined whether pharmacological interference with ANO6 scramblase activity could be used to restrict lipids delivery in vivo and to suppress PDAC growth. A recent cryo-EM study reported a lipid scrambling groove harboring a lipid trail which is also the binding pocket for established ANO6 scramblase inhibitors niclosamide and 1PBC. Using the coordinates of Niclosamide bound to ANO6 (PDBID: 8SUR) as a template, the 3D conformers of Clofazimine were overlaid and determined the energetically minimized pose of Clofazimine in a protein-bound complex (FIG. 6A). The resultant structure highlights the similarity in binding mode between Clofazimine and Niclosamide, namely halogen insertion into a protein sub-pocket near K370. Clofazimine in this pose is making 187 hydrophobic contacts with 13 amino acid residues in the ANO6 cleft between transmembrane domains 1 and 6. Previous studies have established niclosamide and clofazimine block formation of TMEM16F-dependent syncytia induced by SARS-COV-2. To further validate the specific inhibitory effect of clofazimine on PtdSer externalization in PDAC tumors, we injected PtdSer-binding agents into tumor-bearing KPC mice (FIG. 6B, C). A newly developed PtdSer targeting fusion protein (betabody, KL5c) constructed by linking PtdSer-binding domain V of beta2 glycoprotein I to the Fc-fragment of IgG2a binds to externalized PtdSer with high affinity (˜1 nM) as determined in cryosections of murine PDAC (FIG. 6B and 14A, B) co-localizing with cytokeratin-expressing PDAC cells or podoplanin-positive CAFs. Externalized PtdSer was also readily detected in KPC tumors in vivo (FIG. 6B) using a cell membrane non-permeable fluorescent PSVuc-794-labelled probe. Treatment with clofazimine completely suppressed detection of externalized PtdSer in PDAC using these two chemically unrelated probes supporting the specificity of clofazimine as a PtdSer scramblase inhibitor.

Because ANO6 is expressed in both CAFs and PDAC cells, we next tested the activity of ANO6 scramblase inhibitors in CAF monocultures and in co-cultures with DPAC cells. As expected, treatment of control CAFs with the calcium ionophore ionomycin increased cytoplasmic calcium and led these cells to externalize PtdSer to the PM. However, ionomycin did not cause externalized PtdSer in ANO6-depleted CAFs (ANO6-KD) or in CAFs pre-treated with the ANO6 inhibitor niclosamide [74] (FIG. 6D, E). Further, co-culture of human CAFs with Panc-1 cells resulted in robust increase in scramblase activity, reflected in a large increase in PtdSer exposed on the CAF PM (FIG. 6F, G). Importantly, we noted that this increase was observed only in control CAFs (ANO6-proficient) engaged in physical contacts with DsRed-tagged Panc-1 cells, but not in ANO6-KD CAFs, CAFs not engaged in physical contacts with PDAC cells, or in CAFs treated with the ANO6 inhibitor niclosamide (FIG. 6F, G). Similarly, both the SOCE inhibitor BTP2 and clofazimine suppressed PtdSer externalization in CAFs co-cultured with Panc-1 cells (FIG. 6H, I).***

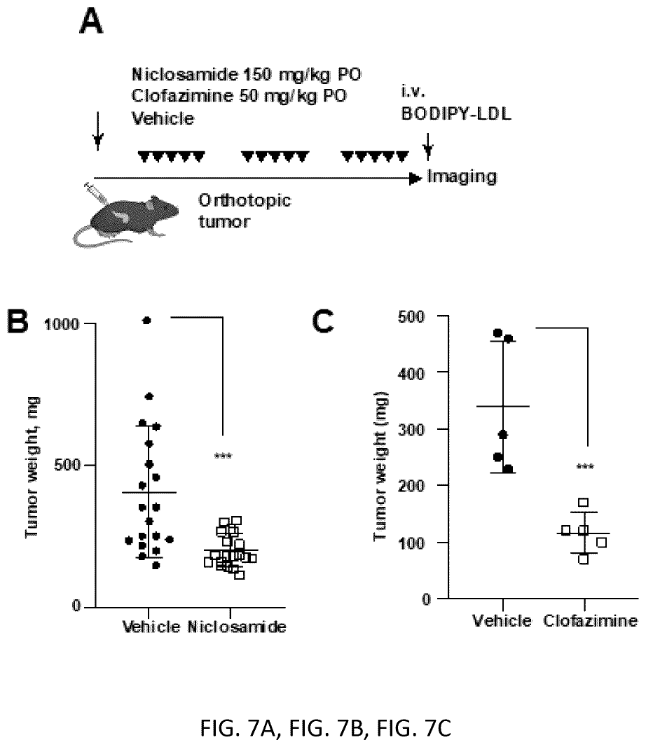

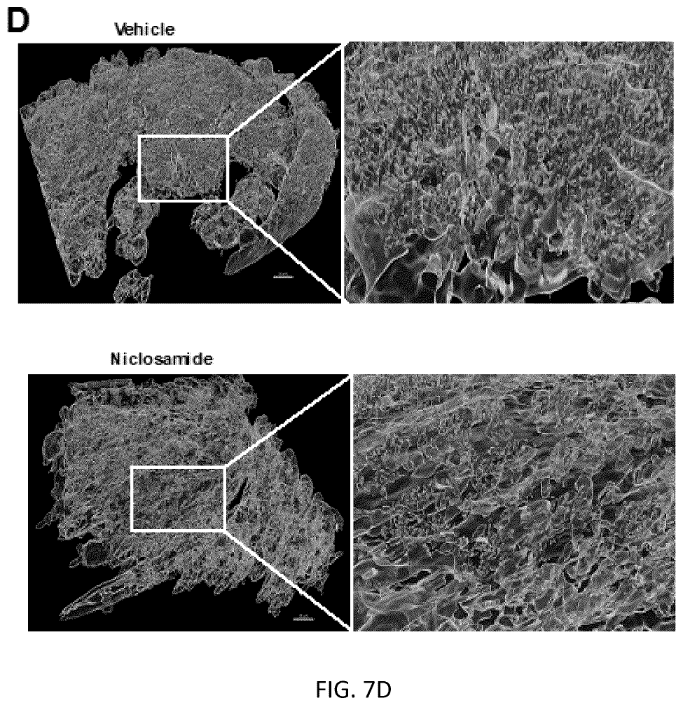

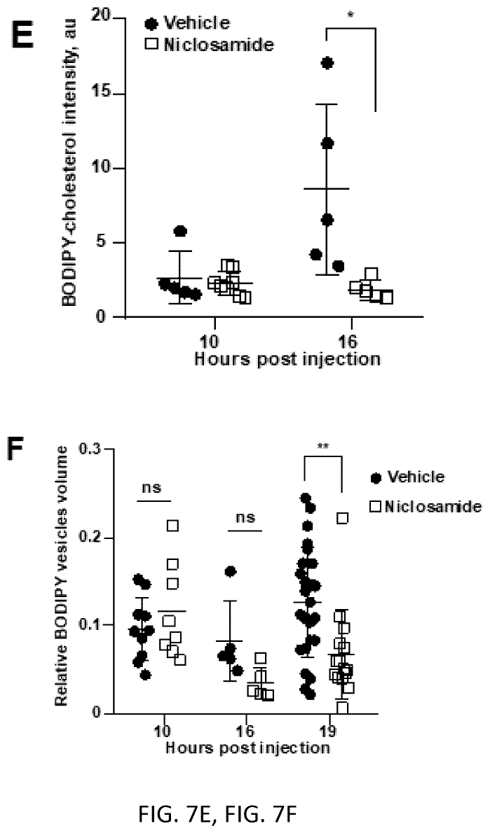

Critically, it was also determined that ANO6 blockade antagonized in vivo growth of orthotopic grafts of pancreatic cancer cells with intact NSDHL via blockade of lipid delivery. Treatment of orthotopic syngeneic KPC3 pancreatic tumors with the ANO6 inhibitors niclosamide (FIG. 7A, B and 14C) or clofazimine [78] (FIG. 7A, C; 14D) significantly reduced tumor weights. To evaluate cholesterol uptake by cancer cells, we intravenously administered LDL particles carrying BODIPY-cholesterol into niclosamide or vehicle-treated mice bearing DsRed-tagged PDAC KPC3 tumors. Treatment with niclosamide suppressed BODIPY-cholesterol accumulation in the DsRed-tagged PDAC KPC3 cells, while the control vehicle-treated tumors showed 6-fold progressive enrichment of BODIPY-cholesterol fluorescence in cancer cells at 16 hours post intravenous injection (FIG. 7D). Analyses of intratumoral vesicles containing BODIPY-cholesterol revealed progressive cholesterol accumulation in vehicle-treated orthotopic tumors at 19 hours post injection, while a significantly lower accumulation was observed in niclosamide treated mice (FIG. 7E, F).

### vii. ANO6-Regulated CAF Trogocytosis is Immunosuppressive Against Native or CAR-Bearing CTLs

The results describe how ANO6 regulates trogocytosis in CAFs. As pro-tumoral CAFs are also known to be highly immunosuppressive (e.g., against CD8-positive CTLs, and because trogocytosis acts as a mechanism by which CTLs acquire the excess of cholesterol and other lipids can trigger lipotoxic ER stress, known to result in CTL exhaustion, it was investigated if ANO6-regulated CAF membrane trogocytosis also played a role in T cell dysfunction in PDAC.

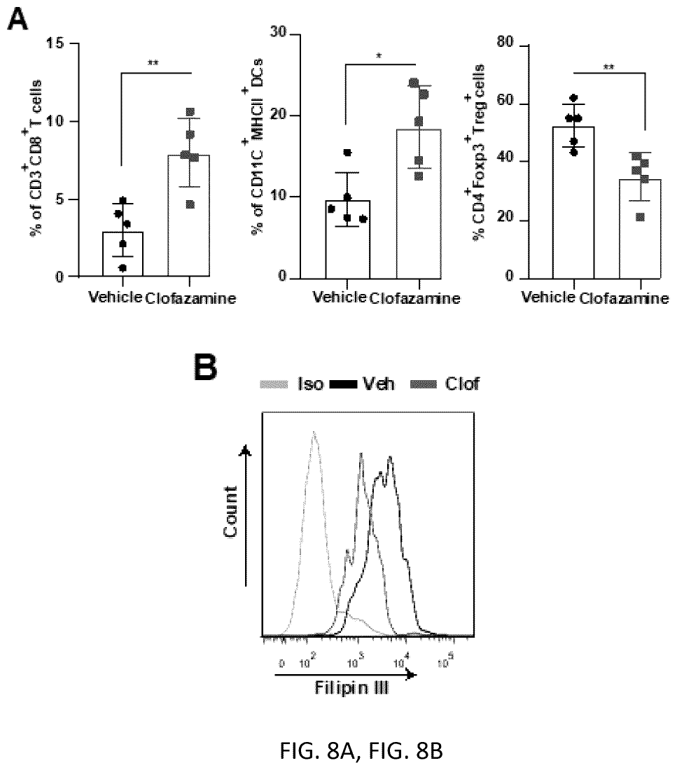

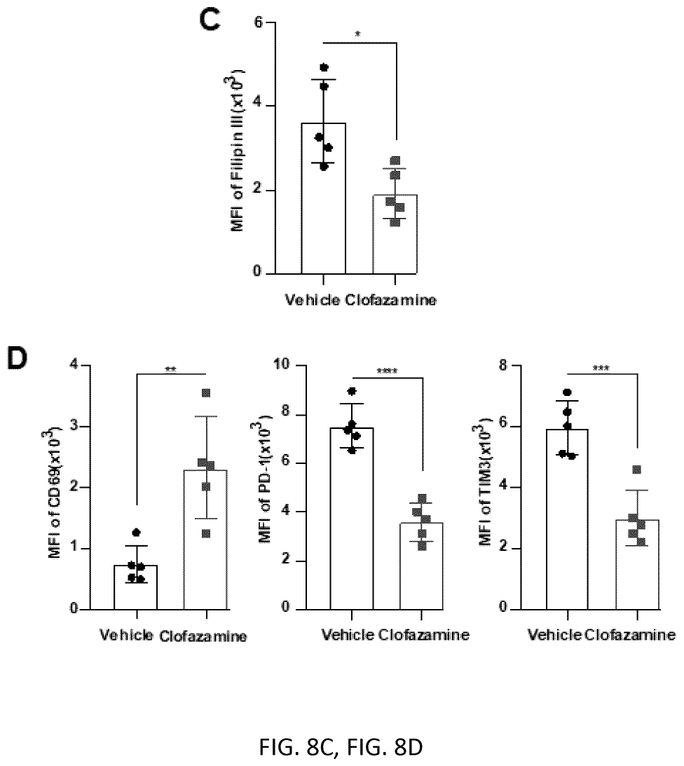

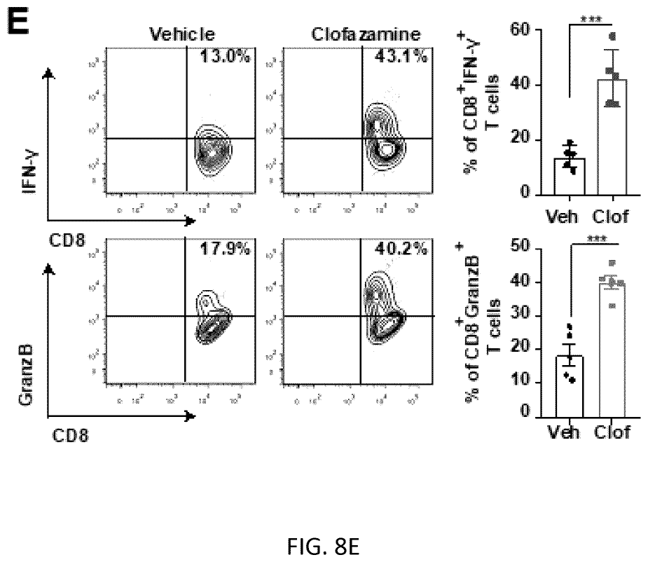

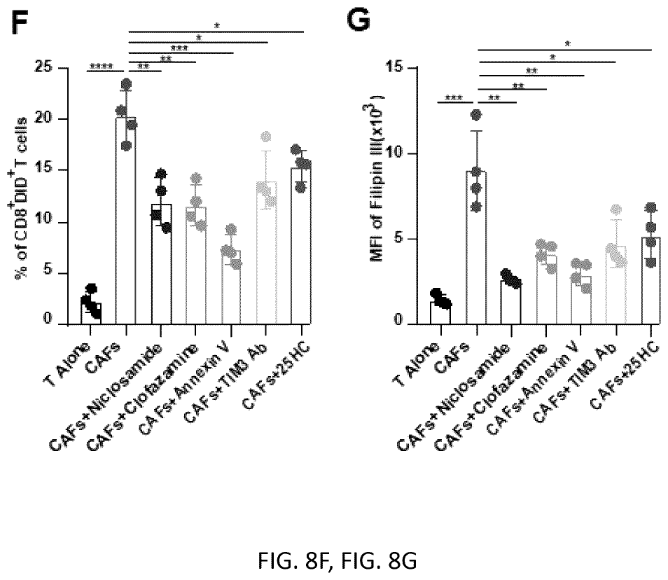

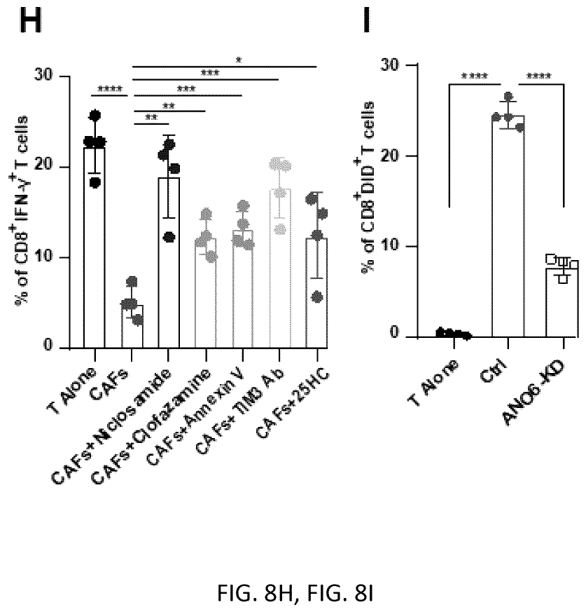

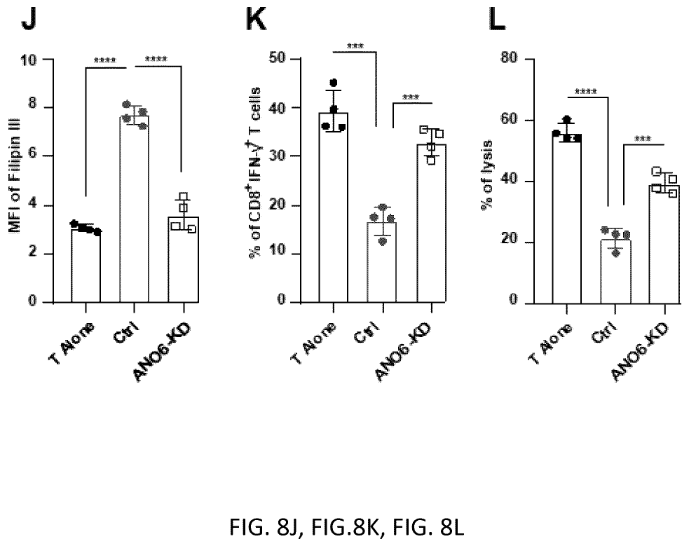

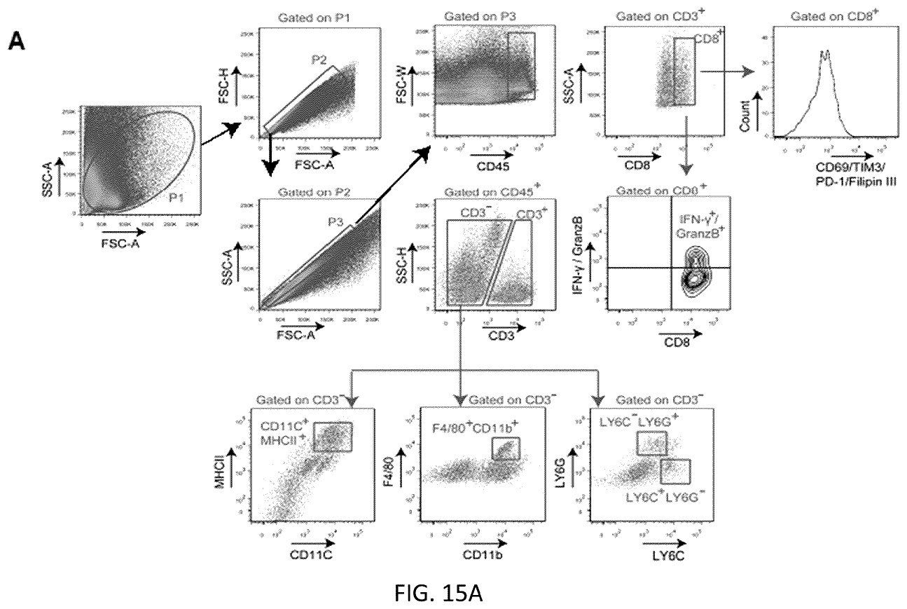

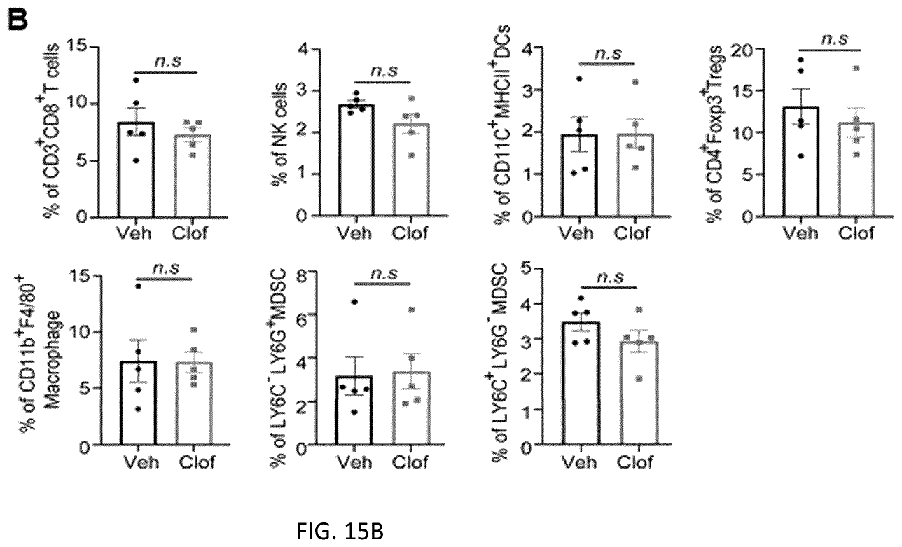

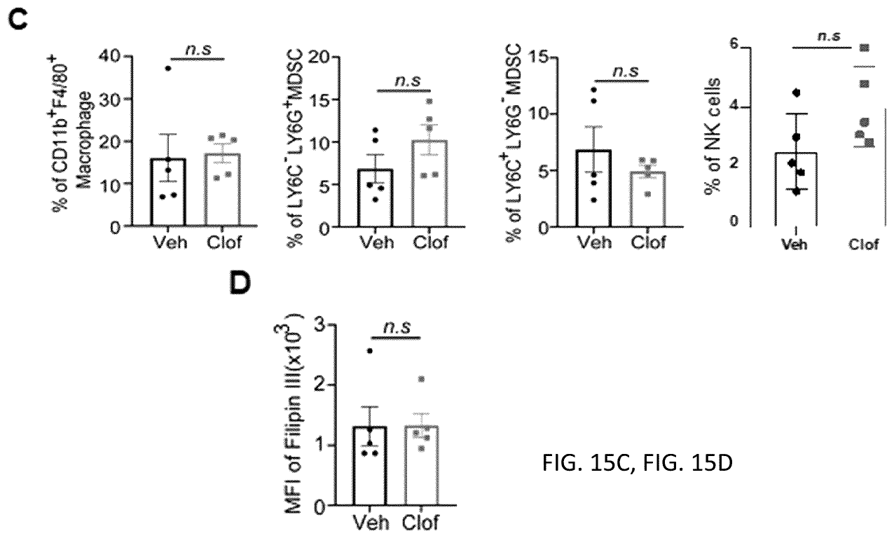

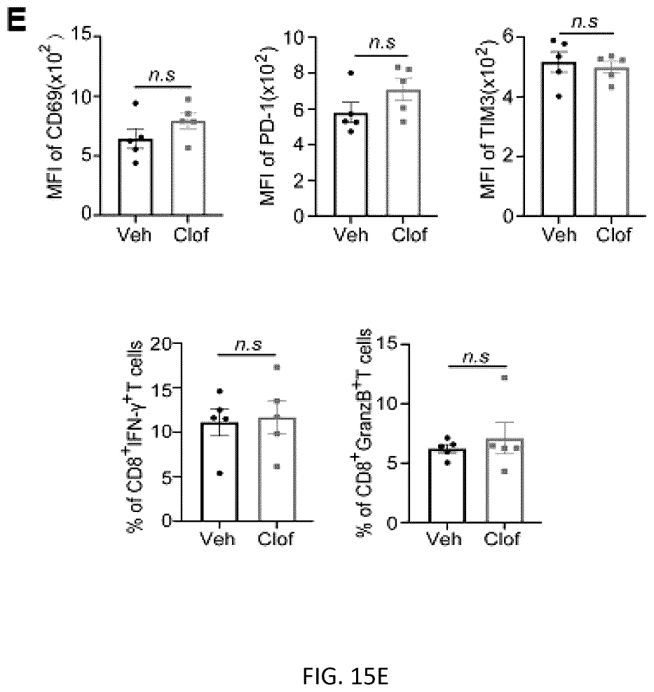

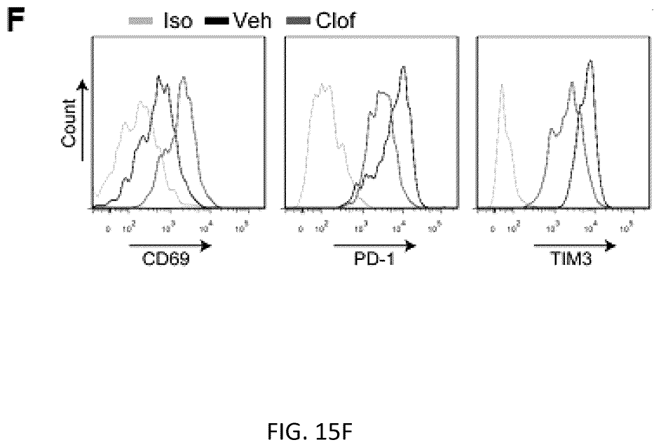

To this end, immunoprofiling analyses were conducted (FIG. S7A) of tumor and splenic tissues harvested from the PDAC tumor-bearing mice treated for 3 weeks with the ANO6 inhibitor clofazimine and compared these to vehicle-treated controls (FIG. 7C). No clofazimine-induced changes were found in the frequencies of immune cells in the spleen (FIG. 15B). Further, clofazimine also did not change the numbers of intratumoral CD11b+F4/80+ macrophages, LY6G+ granulocytic or LY6C+ monocytic myeloid-derived suppressor cells, or the percentage of detected NK cells (FIG. 15C). However, PDAC tumors from clofazimine-treated mice included significantly fewer regulatory T cells (Tregs) and increased numbers of intratumoral CD8+ T cells as well as type I conventional dendritic cells (FIG. 8A).

Next CD8+CTLs were characterized from these mice. Notably and in accord with the hypothesized notion that ANO6 drives pro-tumoral CAF function via regulation of heterotypic cell-cell lipid transfer, clofazimine treatment decreased cholesterol levels in intratumoral CTLs (FIG. 8B,C), but failed to do so in splenic CD8+ T cells (FIG. 15D). This observation suggests that clofazimine can only affect cholesterol levels in T cells that are in direct contact with malignant cells and/or CAFs. Accordingly, clofazimine-induced changes in markers of T cell effector function or exhaustion were not seen in splenic CD8+ CTLs (FIG. 15E). However, intratumoral CTLs from mice treated with this ANO6 inhibitor displayed increased levels of expression of known T cell activation markers such as CD69, interferon γ (IFN-γ), and Granzyme B (FIG. 8D, E), and decreased expression of known exhaustion markers, such as PD-1 and TIM3 (FIG. 8D and 15F).

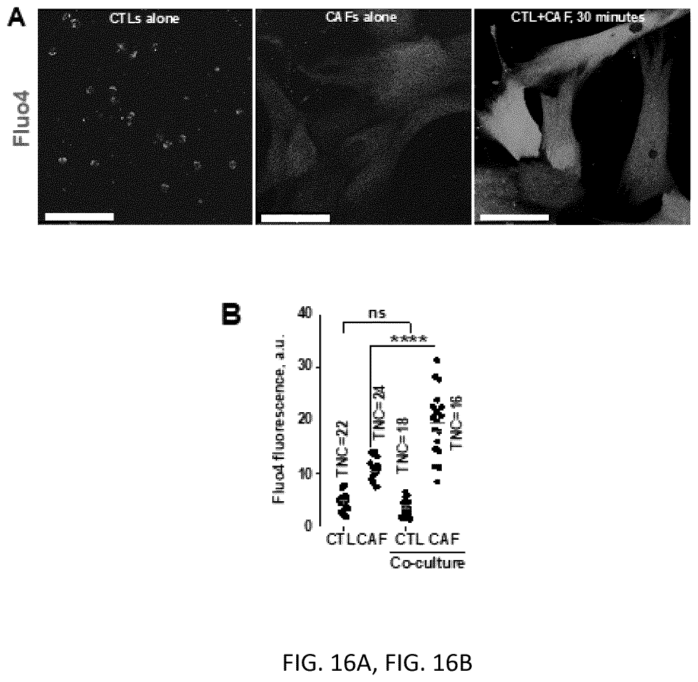

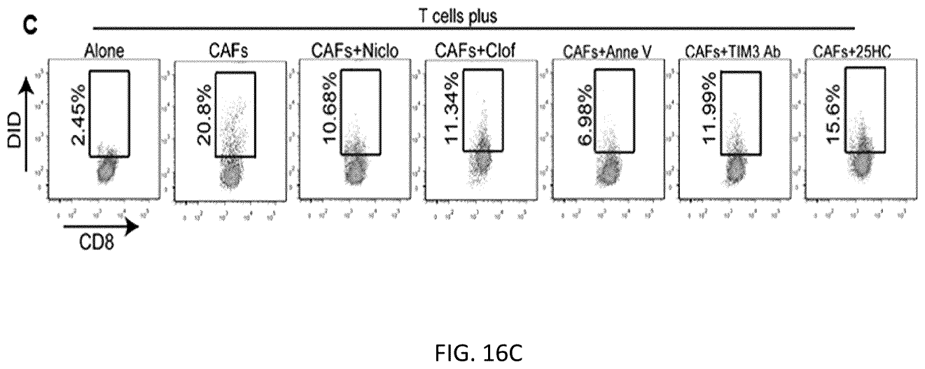

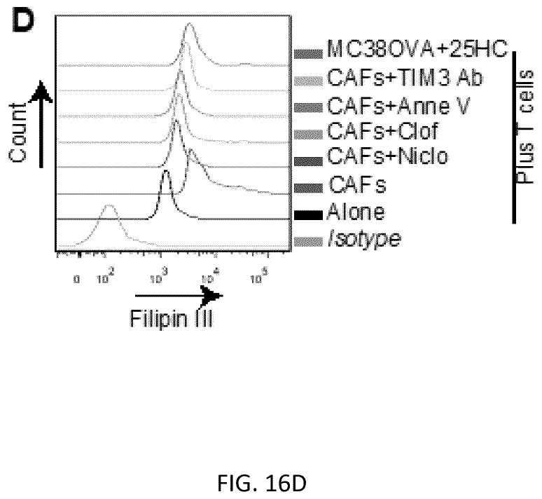

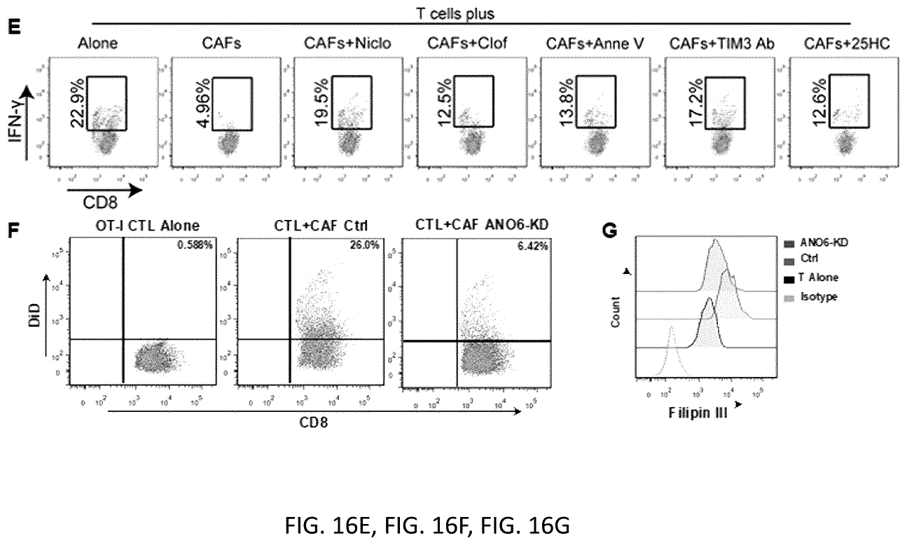

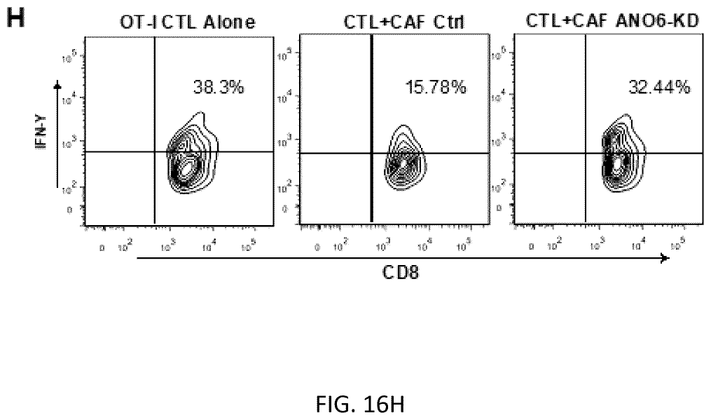

The addition of OT-1 CTLs carrying a transgenic T cell receptor known to target an ovalbumin epitope to CAF1 cells in co-cultures induced rapid activation of calcium influx (FIG. 16A, B) indicating the ability of CTLs to activate SOC channels in CAFs in a manned similar to PDAC cells (FIG. 5A, B). These results suggest that, similar to the heterotypic CAF-PDAC cell contacts, CAF-mediated cholesterol transfer into intratumoral CTLs decreases the anti-tumor function of CTLs. To test this hypothesis and investigate this mechanism in a more defined cellular model, we conducted a trogocytosis assay with CAFs labeled with the lipid-soluble DiD dye and co-cultured with OT-I CTLs. As a control, blockade of trogocytosis with 25-hydroxycholesterol acting on acceptor CTLs, effectively inhibited the transfer of DiD from CAFs to CTLs as expected (FIG. 8F and 16C). Importantly, blockade of PtdSer transfer from CAFs to CTLs, either with a neutralizing antibody against TIM3 (the major receptor for externalized PtdSer and a key regulator of trogocytosis in T cells) or with recombinant Annexin V, significantly reduced CAF PM uptake (labeled by DiD) by CTLs. In support for the critical role of ANO6 in CAF membrane trogocytosis, pre-treatment of ANO6+ CAFs with ANO6 inhibitors, clofazimine or niclosamide, notably suppressed the measured transfer of the lipophilic dye (FIG. 8F and 16C).

Similarly, this blockade of CAF trogocytosis also prevented the increase in the levels of cholesterol in the acceptor CTLs (FIG. 8G and S8D). These results indicated that CAFs transfer membrane-incorporated cholesterol onto CTLs in an ANO6-PtdSer-TIM3 dependent manner. Importantly, these inhibitors of the ANO6-PtdSer-TIM3 pathways and 25-hydroxycholesterol also reduced the ability of CAFs to inhibit IFN-γ production by the CTLs (FIG. 8H and 16E). These pharmacological studies were further corroborated in CTL/CAF co-cultures; ANO6-KD CAFs exhibit reduced DiD uptake (FIGS. 81 and 16F), reduced CTL cholesterol (FIG. 8J and 16G), increased CTL expression of IFN-γ (FIGS. 8k and 16H), and increased OT-1 CTL-dependent lysis of target MC38-OVA cancer cells (FIG. 8l) compared to control, ANO6-expressing CAFs.

Collectively, these results are consistent with our above-postulated hypothesis proposing a key pro-tumoral role for CAF-expressed ANO6, as data suggest that in addition to nurture cholesterol-auxotroph PDAC cells, CAFs drive CAF-to-CTL trogocytosis and thus uphold CTLs immunosuppressed while ensuring high cholesterol levels in both PDAC and CTLs within the pancreatic tumor milieu and resulting in smaller pancreatic tumors (FIG. 8).

### 3. Discussion

The unique PDAC microenvironment has long been recognized as nutrient-poor and immunosuppressed, linked to an unusually high proportion of pro-tumoral functioning CAFs to tumor cells (estimated as at least 5 to 1). In this study, trogocytosis was identified as a versatile mechanism by which CAFs simultaneously support PDAC cancer cell growth and suppress CTL anti-tumor responses. It was also shown that ANO6 scramblase activity is critical to initiate trogocytosis via exposure of PtdSer on the outer PM leaflet of pro-tumoral CAFs. It was further found that direct heterotypic cell-cell contact (e.g., between PDAC cells and CAFs) initiates this pathway by triggering STIM1-Orai signaling at CAF intrinsic ER-PM junctions localized at points of these cell-cell contacts in proximity to ANO6. STIM1, in turn, is known to cause activation of Orai channels. This tripartite association leads to an influx of calcium molecules that is needed to bind and activate ANO6.

Because of the oxygen-poor microenvironment, metabolic parasitism for cholesterol and other nutrients is a common feature of the most lethal basal subtype of pancreatic adenocarcinoma, making it a high priority to identify factors essential for this parasitism. For lipids, prior studies have assessed the impact of blocking lipid uptake receptors such as LDLR, or scavenge receptors such CD36 and SCARB1. The efficacy of a single-point blockade in these studies may have been limited by the apparent redundancy of lipid uptake mechanisms, which may underlic the observed cancer cell evasion. An alternative possibility, raised by this study, is that direct trogocytosis-dependent transfer from CAFs to PDAC cancer cells represents the dominant source of uptake for exogenous cholesterol and other lipids carried from blood to the tumor interstitial fluid. To this end, ANO6 is identified as a drugable and calcium-regulated target with the potential to disable this key mechanism. The results could also explain the cancer-promoting effect of CAFs in pancreatic desmoplasia, not only in nurturing the cancer cells by providing these with direct access to lipids, but also in repelling the anti-tumor activity of immune cells.

An intriguing element of the mechanistic analysis in this study is that the sustained trogocytic heterotypic cell-cell contacts, between cancer cells and CAFs, are also highly reminiscent of the ones described for immunological synapses. Specifically, these synapses have been described as structured membrane microdomains in which ion channels, pumps and mitochondria coalesce to drive efficient T cell activation. It was surprising to find that similar synapse-like contacts are formed between the cancer cells and CAFs in which polarized structures including ER-PM contacts sites enriched for STIM and Orai channels mediate contact-dependent calcium influx from the extracellular space to the cytosol. Such contact-dependent calcium signaling via Orai is prerequisite for the scramblase activity of ANO6. Of note, the Orai inhibitor zegocractin (CM4260) is being tested in clinical trials of acute pancreatitis (e.g., NCT03401190, NCT04195347; clinicaltrials.gov), a strategy which could be deployed to disrupt the PDAC-CAF heterotypic interactions and CAF trogocytosis.

From this perspective, it is perhaps not surprising that heterotypic CAF-CTL cell-cell interactions exert immunosuppressive effects upon T cells, similar to those elicited by membrane transfer from malignant cells. While CTLs require cholesterol for proliferation and activation, it has been demonstrated that excessive uptake of cholesterol in the tumor microenvironment induces CD8-positive T cell exhaustion. Whereas the role of PtdSer receptor TIM-3 has been previously suggested to play a role in T cell inactivation, the new evidence derived from pharmacological and genetic experiments (FIG. 7, 17) indicates that ANO6 and externalized PtdSer directly regulate the immunosuppressive function of CAFs, which is the focus of this study. Since ANO6 is also expressed in cancer cells, it is likely that a similar trogocytosis-mediated mechanism could also antagonize CTLs that are directly engaged in heterotypic cell-cell contacts with PDAC cells.

Finally, antimicrobials such as niclosamide and clofazimine, initially discovered as blockers of COVID19-induced syncytia, were effective, among other known targets, in photocopying ANO6 loss in CAFs and in abrogating PtdSer externalization, including controlling the growth of PDAC xenografts in vivo. Due to their fibroblastic normalizing effect, evidence by inhibition of CAF-mediated delivery of lipids to both nurture cancer cells and uphold immunosuppression, we propose that these ANO6 inhibitors, albeit indirect yet also well tolerated and inexpensive may constitute future highly valued anti-cancer therapies, especially for CAF-relevant solid tumors such as pancreatic cancer.

### 4. Materials and Methods

### i. Animal Studies

All animal protocols were reviewed and approved by the Institutional Animal Care and Use Committee of Fox Chase Cancer Center. C57BL/6J mice were purchased from Jackson Laboratory and maintained at the FCCC Animal Facility. C.B-17.icr SCID mice were purchased from Taconic, NY.

For the xenograft study, anesthetized 6-8-week-old C.B-17.icr SCID mice (Taconic, NY) were injected into the tail of pancreas with 5×10⁵ of NSDHL-depleted Panc-1 cells (CRISPRi-mediated knockdown) in 30 μL of serum-free DMEM. For co-implantation experiments, 5×10⁵ Panc-1 cells were mixed with 1.5×10⁶ of CAFs in 30 μL of serum-free DMEM.

Syngeneic 5×10⁵ KPC3 cells were injected into the tail of pancreas of anesthetized C57B1/6J mice. Treatment with ANO6 inhibitors niclosamide or clofazimine commenced on 2 days post-implantation: niclosamide (150 mg/kg of mouse body weight, Cayman #10649), clofazimine (50 mg/kg, MedChem Express), or corn oil (50 mg/kg, Sigma #C8267) as vehicle were given by oral gavages 5 days/week for 3 weeks. Mice were kept under defined-flora pathogen-free conditions at the AAALAC-approved Animal Facility of the Fox Chase Cancer Center, Philadelphia, PA. Mice of both genders, equally distributed, were used for the experiments.

For phosphatidylserine imaging in autochthonous pancreatic tumors, tumor bearing 6-7 week old KPC mice (genotype Pdx1-Cre; KrasLSL-G12D; Trp53f/f) were given 2 daily doses of clofazimine or corn oil vehicle and injected with 50 mcg of biotinylated betabody KL5c [75] in 0.1 ml of saline followed in 5 hours by necropsy and snap freezing of tumor tissues. For PSVue-794 imaging, animals were treated with clofazimine or vehicle as above and injected intravenously with 0.075 ml of 1 mM solution of PSVuc-794 (cat #P-1001, Molecular Targeting Technologies, West Chester, PA). Animals were euthanized 24 hours post PSVue-794 injection, whole livers and tumors were imaged on Odyssey infrared scanner (Li-COR).

### ii. In Vivo Fluorescent Low Density Lipoprotein Imaging by Multiphoton Microscopy

Mice with established orthotopic tumors roughly 21 days post implantation were anesthetized with 1.5-2% isoflurane/oxygen mixture maintained for the duration of imaging. Pancreatic tumors were exposed through a 1 cm incision on the left flank of the mouse placed on a 35° C. heating pad (Kent Scientific). Intensity thresholds were set up to distinguish signal from background fluorescence, and areas positive for DsRed, indicative of tumor cells, were selected for 3D confocal imaging using a 25× objective in the Leica SP8 Dive multiphoton imaging system (Leica Microsystems), prior to the intravenous administration of 150 μL of LDL carrying BODIPY-cholesterol and at 0.5, 1, 5, 16, and 19-hour interval post injection with XY resolution 0.867 um/px and Z distance 2 μm. For each time interval, 2-3 mice were imaged for 0.5-2 hours under continuous anesthesia. At the end of each imaging session, the animals were euthanized, and tissues were collected. The acquired 3D stacks were analyzed using Imaris 10.0.0 software (Oxford Instruments, UK).

For measurements of BODIPY-cholesterol uptake by tumors in FIG. 6D, tumor volumes were defined using “surface option” in Imaris package with surface grain size 0.867 μm, intensity threshold and size exclusion 300 voxel to exclude non-specific speckles. Volume outside of the tumor within 20 um of its boundary was defined using “distance transform” option in Imaris software. Cholesterol volume was identified using “surface option” in Imaris with surface grain size 0.867 um, automatic intensity threshold and size exclusion set at 10 voxel. Mean intensity of cholesterol volume co-localized with tumor volume or volume outside of tumor was measured and ratio inside intensity/outside intensity was calculated.

For measurements of BODIPY-cholesterol vesicles in tumors in FIG. 6E, tumor volumes were defined as above. The volumes of BODIPY-cholesterol-positive vesicles were defined using a surface grain size of 0.1 μm and background subtraction with 3 μm sphere, automatic intensity threshold and exclusion of objects below 26 voxels. The volume of vesicles inside the tumor were expressed as a percentage relative to tumor volume.

### iii. Cell Lines

PDAC cells were obtained from ATCC and maintained in DMEM supplemented with 10% v/v FBS, 2 mM L-glutamine and 100 mg/ml Penicillin/Streptomycin. Murine pancreatic carcinoma cell lines were isolated. All cell lines were regularly tested for Mycoplasma, as determined by PCR detection methods, and all lines tested negative. Human CAF1 cell line was derived from the residuals of PDAC surgical samples in accord with the Fox Chase Cancer Center IRB-approved informed consent from patients who donated samples for research purposes. An additional similarly harvested human pancreatic CAF line (referred to as CAF37) was obtained.

### iv. Functional Assessment of Fibroblastic/ECM Functional Units; Fibroblast-Derived 3D Matrix Cultures

Functional fibroblastic cell 3D culture units are needed to ensure the pathophysiological signal reciprocity between fibroblastic cells and self-generated ECM is sustained ex vivo. These units were generated using known methods. Briefly, confluent cultures of human pancreatic fibroblastic cells, harvested from surgical samples and immortalized via hTERT overexpression, were supplemented daily with freshly-prepared ascorbic acid (50 ug/mL) for a period lasting 5 days. The resulting fibroblastic/ECM 3D functional units were used for the phenotypic characterization of the units and to report on the pro-tumor vs. tumor-suppressive (normalized) statuses of the experimental fibroblastic/ECM units. For this, lysates obtained from the assorted units were resolved using SDS-PAGE and traits were gauged via western blot; testing for constitutive integrin and PI3K activities depicted by phosphorylated FAK and AKT respectively.

Of note, the quality of ex vivo 3D functional units was measured via ECM thinness (indicative of intact fibrillogenesis), while loss of ECM anisotropy (e.g., low parallel ECM fiber alignment) indicated effective unit normalization; the latter was measured via indirect immunofluorescence of fibronectin fibers analyzed using monochromatic 2D image projections. Images were uploaded to ImageJ (SCR_003070) and ECM fibers were analyzed with the OrientationJ plugin SCR_014796//bigwww.cpfl.ch/demo/orientation/. Numerical fiber orientation/angle outputs were normalized by setting each image's mode angle to 0°, thus correcting angle spreads/distributions to fluctuate between −90 and 90. Angle distributions, for each experimental condition, corresponding to a minimum of three experimental repetitions and five image acquisitions per condition, were plotted and their standard deviations calculated. The percentage of fibers oriented at 15 degrees from the mode (ranging from −15° to 15°) was determined for each image-obtained data to inform the presented graphs.

### v. Plasmid Transfections and CRISPRi-Mediated Gene Silencing

Transfection of GFP-MAPPER (117721, Addgene, Watertown, MA), mCherry-ANO6 (62554, Addgenc, Watertown, MA) or STIM1-YFP (19754, Addgene, Watertown, MA) were done by mixing 2.5 μg of plasmid DNA with 7.5 μl of TransIT-X2 reagent (Mirus, Madison, WI). The mixture was added to 10⁵ CAF or PDAC cells cultured in serum-free and antibiotic-free media overnight.

For CRISPRi, we used an all-in-one lentiviral CRISPRi plasmid containing a nuclease-dead Cas9 (dCas9) fused to the transcriptional repressor domain KRAB and a gene specific gRNA [42]. The top 3 best predicted gRNA were chosen and individually cloned into the all-in-one lentiviral vector, CRISPRi-Puro modified from the Addgene plasmid #71236. Lentiviruses were generated by transfecting the CRISPRi-Puro plasmid, along with the packaging plasmids psPAX2 (Addgene plasmid #12260) and pMD2.G (VSV-G envelope; Addgene plasmid #12259) with X-tremeGene9 transfection reagent (#6365787001, Sigma-Aldrich) into 293T cells in serum free/antibiotic free media overnight. The following day, media was replaced with complete media containing FBS and media containing lentiviruses were collected at days 2 and 4 post-transfection. The lentiviral media was then filtered through a 0.45 uM filter (#HAWP14250, Millipore) and used to transduce target cells, by culturing cells in lentiviral media plus 10 ug/mL Polybrene (#sc-134220, Santa Cruz Biotechnology). After 24 hours, lentiviral media was replaced with complete media and cells were selected 48 hours later with puromycin (specific for each cell lines) for 14 days. Surviving cells were subsequently expanded and knockdown of target genes was confirmed at the protein and transcriptomic levels.

### vi. Generation of ANO6-mCherry Mutant CAFs

Mutations in the ANO6 sequence of plasmid ANO6-Plvx-mCherry-cl (Addgene #62554) were created using the Q5® Site-Directed Mutagenesis Kit (NEB E0554S) based on the established functional scramblase activity data. Primers were designed with the NEBaseChanger tool (//nebasechangerv1.neb.com/). The intended substitution for each mutation was confirmed by Sanger sequencing (Azenta) and the mutated plasmids were transfected into HEK293T cells. The resulting viruses were filtered and transduced into CAF ANO6 CRISPRi-depleted cells. After growth and expansion, cell lines were selected by flow cytometry for mCherry expression.

### vii. Analyses of Protein Expression by Western Blotting

Cultured cells were lysed in RIPA buffer (#24928, Santa Cruz) with phosphatase inhibitor (#1862495, Thermo Fisher Scientific) and protease inhibitors (#1861278, Thermo Fisher Scientific) on ice and cleared by centrifugation (15 min at 17,000 g). The protein concentration was measured with a Pierce BCA Protein Assay Kit (#23225, Thermo Fisher Scientific). Proteins were separated on 4-12% Bis-Tris Protein gels (Invitrogen) and then horizontally transferred to the Immobilon-FL PVDF membrane (#IPFL00010, Millipore). Primary and secondary antibodies were used at the concentrations indicated below according to manufacturer's instructions. The density of obtained bands was quantified with Image J software (FIJI).

### viii. Lipid Droplets Fluorescence Measurements; Normalized CAF Assessment

Quantification of lipids droplets accumulated in PDAC or CAFs cells (5000 cells per well in a 96-well plate) was conducted using the Lipid Droplets Fluorescence Assay Kit (Cayman #No. 500001 or AAT Bioquest #22730) according to the manufacturer's instructions. The fluorescence intensity, indicative of lipid droplet accumulation, was measured using a plate reader, also following the manufacturer's recommended settings.

### ix. Cholesterol Measurement

Cholesterol level in PDAC or CAFs cells were measured by Amplex Red Kit (#A12216, Life Technologies). Briefly, 500,000 cells were seeded in a 6-well plate and cholesterol was measured in cell lysate or supernatant according to the manufacturer's instructions. BCA test was performed in parallel for normalization.

### x. Phosphatidylserine Externalization Assay

CAFs were seeded in 24-well glass bottom plates (cat. #81156, Ibidi, Fitchburg, WI) at a density of 20,000 cells per well. Cells were then treated with niclosamide (1 μM) for 2 h. After one wash with FBS-free media at RT, cells were incubated with 250 μl of 1:100 annexin XII and 1:200 propidium iodide (pSIVA Abcam #ab129817), with or without 10 μM ionomycin. Cells were then incubated (5 min 37C) and imaged by fluorescence microscopy using a Nikon A1 camera (Nikon) linked to a Nikon Eclipse Ti2-E Inverted Microscope Imaging System (Nikon) for each channel. Fifteen images per well were acquired.

### xi. Colony Formation Assay

10³ PDAC cells were plated in 2.5 ml of media on 6-well plates with or without 105 CAFs in DMEM supplemented with 10% v/v FBS and 2 mM L-glutamine with 100 μg/ml Penicillin/Streptomycin. After 24 hours, the old media was replaced with new media according to experimental conditions. After 10 days, the cultures were washed with PBS twice and fixed in 10% methanol 10%/acetic acid solution for 15 minutes at RT and stained for 20 minutes with 0.4% Crystal Violet in 20% ethanol. Plates with colonies were scanned and images were analyzed with ImageJ software using the Colony Area plugin.

### xii. Annexin V Apoptosis and Viability Assays

PDAC cells were pre-labeled with eFluor670 (CPD670) according to the manufacturer's instructions (#65-0840-85, eBioscience) and plated either alone or in co-cultures with CAFs at 10⁵ cells in 2 ml per well in 6-well plates in DMEM supplemented with 10% FBS, 2 mM L-glutamine, and 100 uμg/ml Penicillin/Streptomycin. After 24 hours, the medium was replaced with indicated experimental media supplemented with 10% FBS, 10% lipid-depleted serum (LDS), or LDS supplemented with purified donor LDL (#360-10, LEE Biosolutions, Maryland Heights, MO, USA). After 72 hours, cells were harvested by trypsinization and stained with Annexin V-FITC (#640906, Biolegend) or Annexin V-PE (#640908, Biolegend) according to the manufacturer's instructions. Dead cells were determined by DAPI staining (#D9542, Sigma-Aldrich), fluorescence was measured using BD LSRII flow cytometer and analyzed with FlowJo software.

For direct enumeration of viable cells in co-cultures with CAFs, we seeded CAFs at 12,000 cells per well in 96-well plate (#3603, Corning) in DMEM/10% FBS. The next day, the media was replaced with 10%/LDS/DMEM and 4,000 DsRed-tagged Panc-1 cells were plated on the top of CAFs at 3:1 ratio. After 96 hours, fluorescence of viable DsRed-positive Panc-1 cells was measured using Spark Multimode Microplate Reader (Tecan) with parameters set at 540 nm for excitation and 590 nm for detection. To measure the dynamics of cell proliferation in vitro, we used xCELLigence E-Plate (ACEA BioSciences,) as per the manufacturer's recommended protocol. Impedance values were recorded every hour. Data was graphed using GraphPad Prism software.

### xiii. Trogocytosis Assays In Vitro by Flow Cytometry

To label cellular membranes, pairs of lipophilic fluorescent dyes were used according to the manufacturer's instructions in the following combinations: 1) adherent CAFs were stained with 1 μM PKH67 (Sigma-Aldrich), Panc-1 were stained in suspension with 10 μM CellTracker™ orange CMCMR (Thermo Fisher Scientific); 2) CAFs labeled with 0.5 μM BODIPY-cholesterol (Topfluor Cholesterol, #DO-016545, Avanti Polar Lipids) dissolved in 0.1 mM methyl-beta-cyclodextrin (#C4555, Sigma) with 2 μM cholesterol (#C8667, Sigma-Aldrich) in MEM media at 37° C. for 30 min in suspension; murine or human (Panc-1) PDAC cells were stained in suspension with CPD670 (#65-0840-85, Thermo Fisher Scientific). After labeling, CAFs were co-incubated for the indicated time intervals with PDAC cells in 12-well culture plates at ratio of 2:1 in 0.5% DMEM media. Co-cultures of CAFs and PDAC cells for 3 minutes on ice minutes were used as negative controls. In experiments assessing the effect of extracellular calcium on trogocytosis, cells were co-cultured in Phosphate Buffered Saline (PBS) containing calcium and magnesium (D8662, Sigma-Aldrich) or in PBS without calcium and magnesium (D8537, Sigma-Aldrich). After co-cultures, cells were collected and pelleted by centrifugation (5 min, 1500 rpm), resuspended in 10% formalin (Sigma) for 10 min at RT, washed and re-suspended in PBS/1% BSA. Trogocytosis was measured as the acquisition of CAF-specific fluorescence in PDAC cells by flow cytometry on MACSQuant® VYB Flow Cytometer (Miltenyi Biotec) or FACScan analyzer (BD Biosciences). Analysis of dual staining was done using FlowJo software (10.8.1, BD Biosciences). Live cells were gated based on FSC/SSC parameters, then single cells were gated based on SSC-A/SSC-H.

For interference with trogocytosis, CAFs were pre-treated prior to co-cultures for 2 h with recombinant label-free 100 nM Annexin-V (#ab157342, Abcam) or 100 nM Zn-DPA (Molecular Targeting Technologies) and subsequently maintained in co-cultures at the same concentrations.

For trogocytosis between OT-1 and CAF cells, T cells were isolated from splenocytes of OTI mice (strain 021880, The Jackson Laboratory) and stimulated with OVA257-264 (1 μg/ml) for 24 hours as described [24]. CTLs were labeled with DID (DiIC18(5); 1,1′-dioctadecyl-3,3,3′,3′-tetramethylindodicarbocyanine, 4-chlorobenzenesulfonate salt, D7757, Invitrogen) and then co-cultured with CAFs at 5:1 ratio in the presence of 0.05% DMSO as diluent or indicated agents: niclosamide (1 μM), clofazimine (1 μM), anti-TIM3 Ab (4 μg/ml, clone RMT-3-23, BioXCell, Lebanon, NH), or 25-hydroxycholesterol (4 μM, #H1015, Sigma-Aldrich) for 12 hours followed by flow cytometry analysis.

### xiv. Tumor Dissociation and Immune Subset Analyses

Orthotopically implanted syngeneic KPC3 tumor tissues were harvested and digested with 1 mg/mL Collagenase D (Roche, Switzerland) plus with 100 μg/mL DNase I (Roche) in RPMI medium for 45 min with continuous agitation at 37° C. The digestion mixture was passed through a 100 μm cell strainer to prepare single cell suspension and washed with PBS supplemented with 2 mM EDTA and 1% FBS. Single cells were stained with antibodies purchased from BioLegend (San Diego, CA) diluted at 1:200 for staining: CD45-BV785 (cat #103149), CD3-APC (cat #100236), CD8-APC/Cy7 (cat #100714), TIM3-BV421 (cat #134019), CD69-FITC (cat #104506), PD-1-BV605 (#135220), CD11C-PE/Cy7 (cat #117318), MHCII-APC (cat #107615), CD11b-Percp/Cy5.5 (cat #101228), F4/80-BV421 (#123132), Ly6C-BV605 (cat #128036), Ly6G-APC/Cy7 (cat #127624), NK1.1-FITC (cat #156508), CD3-APC/Cy7 (cat #100222), CD8-PE/Cy7 (cat #100722), IFN-γ-APC (cat #505810), Granzyme B-Percp/Cy5.5 (cat #372212), CD3-BV421 (cat #100228), CD4-PE/Cy7 (cat #116016), CD25-APC/Cy7 (cat #, 101908), Foxp3-AF647 in permeabilized cells (cat #,320014).

### xv. Analysis of Cholesterol Level and Cytokines in T Cells

Cholesterol abundance in cell membranes was determined with Cholesterol Detection Assay Kit (Cayman, cat #10009779). Dissociated cells were fixed with fixative solution for 10 minutes, washed and labeled with Filipin III as per the manufacturer's instructions. Labeling with Filipin III was done by incubating in the dark for 30 minutes. After two washes for 5 minutes each, labeling was analyzed by flow cytometry (excitation: 340-380, emission: 385-470). The IFN-γ, Granzyme B and Foxp3 were detected using Foxp3/Transcription Factor Staining Buffer (00-5523-00, Thermo Fisher Scientific). Cells were stained for cell surface markers at 4° C. for 30 min followed by fixation and permeabilization buffer for 30 mins and two washes with permeabilization buffer and incubation with the indicated antibodies for 30 mins, washed with PBS twice followed by flow cytometry analysis.

### xvi. Measurement of Cytosolic Calcium Using Fluo4 Fluorescence Microscopy

CAF (5×10³) and PDAC (104) cells were incubated (separately or in co-culture) on coverslips for 12 hrs before loading with the calcium indicator Fluo4-AM (5 μM; Invitrogen, Eugene, OR) in DMEM supplemented with HEPES and 0.5% FBS and incubated at 37° C., 5% CO2 for 30 min. Cells were washed in DMEM before incubating for an additional 30 minutes at 37° C., 5% CO2 to permit de-esterification of the dye. Fluorescence emissions at 506 nm in response to excitation at 494 nm were monitored for 60 cycles at a frequency of 0.33 Hz using a Leica SP8 laser scanning microscope (Buffalo Grove, IL) at 37° C. with 5% CO2. Intracellular Ca2+measurements are shown as absolute fluorescence obtained from 35-45 cells obtained using Leica LASX software. Averaged results±SEM from at least three independent experiments were analyzed by one-way ANOVA with GraphPad Prism. BTP2 (STIM/Orai inhibitor; Tocris Bio-Techne Corporation, Minneapolis, MN), CM4620 (STIM/Orai inhibitor; MedChemExpress, Monmouth Junction, NJ) and U73122 (PLC inhibitor; AdipoGen, San Diego, CA) were added to the cells at various concentrations for 10 min to determine their effect on cytosolic Ca2+ levels.

### xvii. SOCE Measurement in CAFs

CAF (5×10³) cells were incubated on coverslips for 12 hrs before loading with the calcium indicator Fura2-AM (2 μM; Invitrogen, Eugene, OR) in cation-safe solution (107 mM NaCl, 7.2 mM KCl, 1.2 mM MgCl2, 11.5 mM glucose, 20 mM Hepes-NaOH, 1 mM CaCl2, pH 7.2) for 30 min at 24° C. as previously described [48]. Cells were washed, and dye was allowed to de-esterify for a minimum of 30 min at 24° C. Cells were treated with Vehicle control, 5 M CM462, or 1 μM BTP2 along with 2 μM Thapsigargin for 10 minutes. After Ca2+ levels returned to their baseline, 1 mM Ca2+ was added. Ca2+ measurements were made using a Leica DMI 6000B fluorescence microscope controlled by Slidebook Software (Intelligent Imaging Innovations; Denver, CO). Fluorescence emission at 505 nm was monitored while alternating between 340 and 380 nm excitation wavelengths at a frequency of 0.67 Hz; intracellular Ca2+ measurements are shown as 340/380 nm ratios obtained from groups (20-25) of single cells.

### xviii. GCaMP Tracking in CAF-PDAC Interactions

CAFs were transiently transfected with GCaMP6 and plated on 12-mm circular coverslip for 2 days prior to the experiment. Baseline GCaMP-GFP fluorescence intensity was recorded in CAFs prior to the introduction of mCherry-expressing PANC-1 cells. Fluorescence emissions were monitored overnight at 510 nm in response to excitation at 488 nm was monitored for ##cycles at a frequency of ##Hz using a Leica SP8 laser scanning microscope (Buffalo Grove, IL) at 37° C. with 5% CO2. STIM1-YFP fluorescence was tracked simultaneously. Akino, I think that some comments about how you used the locations of STIM1 and of PANC-1 cells to correlate polarization to heterotypic cell-cell contact sites and calcium content.

### xix. STIM1 Immunocytochemistry

CAF and PDAC cells were cocultured onto 12-mm circular coverslips before fixing with formaldehyde (3.7%; 10 min) and permeabilization with Tween20 (0.5%; PBS). Cells were then blocked with bovine serum albumin (0.5%) before labeling with anti-STIM1monoclonal antibody (1:50 dilution; 30 min; Sigma-Aldrich, Rockford, IL). Cells were then washed before the addition of Alexa Fluor 488-conjugated goat anti-mouse IgG (1:50dilution; 30 min; Invitrogen, Eugene, OR). Fixed and labeled cells were mounted onto slides before visualization with a Leica sp8 confocal laser scanning microscope and analysis with LASX software. Data were analyzed by two-way ANOVAs.

### xx. Timelapse Image Acquisition and Analysis

Images were acquired using an ImageXpress micro confocal microscope (Molecular Devices, San Jose,CA) equipped with environmental control, at 10× magnification (10×, Plan Apo) in wide field mode. Four sites per well with 100 uM between images in both the X and Y direction were acquired, in four wavelengths (transmitted light, CY5 (DRAQ5), FITC (SYBR Green), and Texas Red (DsRed). Total timelapse duration was 96 hours, images acquired at 3-hour intervals for a total of 33 time points. Images were subsequently analyzed using the ‘mutiwavelength scoring’ module (MetaXpress) for image segmentation and quantitation. Image segmentation gated for positive cells using both size and intensity cutoffs. Scoring profiles for single positive (either FITC positive/DsRed negative or FITC negative/DsRed positive) or double positive cells (FITC positive/DsRed positive) were reported for nuclei positive cells (Total cells=nuclei count or W1). Image acquisition was controlled via MetaXpress (64 bit) version 6.7.0.211 (Molecular Devices). Data was exported to MS-Excel for further analysis.

### xxi. In Vivo Cholesterol Transfer Measurement by Flow Cytometry

Tumor dissociation was performed using a gentleMACS Tumor Dissociation Kit (Miltenyi Biotec, Order No. 130-096-730) according to the manufacturer's instructions. Briefly, each tumor was isolated from the animal in a sterile environment and washed in PBS. A piece was taken for histopathology analysis. The rest of the tumor tissue was placed in the dissociation enzyme mix and minced quickly to get pieces 2 mm3 in size. Then the enzyme-tissue mixture was transferred into the gentleMACS C tube and incubated at 37° C. with constant rotation for 40 minutes. After that the tissue was further mechanically processed by the gentleMACS Dissociator. A single-cell suspension was obtained by passing the tissue mixture through a 70 mm cell strainer. Dead cells were subsequently removed by the Dead Cell Removal Kit (Miltenyi Biotec, Order No. 130-090-101). Cell lineage was determined with the lineage markers: CD45 (1:200, #103107, BioLegend), CD11b (1:200, #101235, BioLegend), FAP (1:50, #ab28244, Abcam), EPCAM (1:200, #118212, BioLegend) and PDGFRa (1:80, #135905, BioLegend) in the presence of FcR antibody (1:50, #101301, BioLegend) prior to using other antibodies. Live cells were selected based on propidium iodide staining (#421301, BioLegend). Fluorescence detection and sorting were performed with a BD FACS Aria II flow cytometer.

### xxii. Analysis of Cellular Lipids Using LC-MS

Optima grade methanol, water, acetonitrile, methyl tert-butyl ether, and 2-propanol were from Thermo Fisher Scientific (Pittsburg, PA). Gasses were supplied by Airgas (Philadelphia, PA). Gassware and HPLC vials were from Waters Corp (Milford, MA).

Lipid extraction. Frozen cell pellets (˜1×10⁶ cells) were collected in low retention Eppendorf tube and mixed with 0.6 mL 80% methanol (MeOH) and 20 μL of lipidomics internal standard mix (1:1, SPLASH® LIPIDOMIX #330707: Ceramide/Sphingoid Internal Standard Mixture I #LM6002, both from Avanti Polar Lipids, Alablaster, AL) and kept in dry ice. Samples were pulse sonicated for 30× half-second pulse on ice and kept on ice for 20 min for metabolites extraction. Each tube was then vortexed 3× 30 seconds each. The cell homogenates were moved to a 10 mL glass Pyrex tube with screw cap. The Eppendorf tubes were rinsed with 0.5 mL methanol and added to the same glass tube. 5 mL methyl tert-butyl ether (MTBE) was added to each of the tubes and then tubes were shaken vigorously for 30 min. 1.2 mL water was added to each tube and vortexed for 30 sec each. Centrifugation for 10 min at 1000×g created two phases. The top clear phase was moved to a clean glass Pyrex tube and dried down under nitrogen. 200 μL MTBE/MeOH (1:3 v/v) per 10 mg tissue was used to re-suspend the residue. The sample was spun down at 10,000×g for 10 min at 4° C. and only 100 μL were transferred to a HPLC vial for LC-MS analysis. A pooled sample was created by mixing 20 μL of each re-suspended sample and ran as quality control (QC) every 15 samples. 2 μL injections were made in both positive mode and separately in the negative mode.

### xxiii. Analyses of ANO6 mRNA and Protein Expression in Human Cancers

Data for mRNA expression and patient outcomes for human pancreatic ductal adenocarcinoma, cervical cancer and breast cancer were extracted from the publicly available NCI TCGA program. ANO6 protein expression data were obtained from the NCI Clinical Proteomic Tumor Analysis Consortium (CPTAC) dataset. Analyses to compare the overall survival among the ANO6-high and ANO6-low were performed by comparing the Kaplan-Meyer survival curves with log-rank tests. These were calculated using the R ‘survival’ package.

### xxiv. Single-Cell RNA Sequencing Using 10× Genomics Platform

For single-cell RNA sequencing, KPPC mice with advanced tumors (n=3), were sacrificed at 7-8 weeks of age. Single-cell suspensions were isolated from minced tumors using Miltenyi Biotec gentleMacs dissociator in gentleMACS C Tubes (#130-093-23) and mouse tumor tissue dissociation kit (#130-096-730) as per the manufacturer's instructions. Dead cells were removed by use of Dead Cell removal microbeads (#130-090-101), hematopoietic cells were depleted by CD45 MicroBeads (#130-052-30). The Chromium controller was used to make single-cell droplet with GEM bead. Single-cell suspensions were converted to barcoded scRNA-seq libraries by using the Chromium Single Cell 30 Library, Gel Bead & Multiplex Kit and Chip Kit V3 (10× Genomics, #PN-1000092), loading an estimated 6-12*10³ cells per library per the manufacturer's instructions. Samples were processed using kits pertaining to the V3 barcoding chemistry of 10× Genomics. For each replicate, all tumor samples were processed in parallel in the same thermal cycler. The final libraries were profiled using the Bioanalyzer High Sensitivity DNA Kit (Agilent Technologies) and quantified using the Qubit 2.0 (Thermal Fisher, #Q32851). Each single-cell RNA-seq library was sequenced twice in two lanes of HiSeq 4000 (Illumina) to obtain single-end, 98 bp, approximately 500 million reads per library.

### xxv. Bioinformatics of Mouse Single-Cell RNA Sequencing

For analyses of human PDAC single-cell RNA sequencing, publicly available data for samples CRR034503, CRR034504, CRR034505, CRR034506, and CRR034507 were obtained from Genome Sequence Archive under project PRJCA001063. Single-cell RNA-seq data for advanced KPC mouse pancreatic adenocarcinoma were generated by this study. Sequencing data for each sample were processed using CellRanger 7.1.0 (10× Genomics) to align and quantify sequencing reads using a mouse reference genome (GRCm38). Individual count tables were merged using CellRanger aggr function. Subsequent data analysis was carried out in R 3.5.1 and the Seurat package (v 3.0.2). The following filters were applied to exclude beads without cells and dead cells by imposing a threshold of at least 500 transcripts measured per cell, and less than 5% mitochondrial reads were set as a threshold to exclude dead cells. 20 principal components were used for dimensionality reduction via UMAP with default parameters. Clusters of cells were identified based on a shared-nearest neighbor graph between cells and the smart moving (SLM) algorithm (resolution=0.1). Markers for each cluster were identified by reducing the number of candidate genes to those genes which were (i) at least log (0.25)-fold higher expressed in the cluster under consideration compared to all other clusters and (ii) expressed in at least 10% of cells in the cluster under consideration. For genes passing those criteria, significance between cells in the cluster versus all other cells was calculated using model-based analysis of single-cell transcriptomics (MAST) and adjusted with the Benjamini-Hochberg method. Differentially expressed genes were used as input for gene set expression analysis.

### xxvi. RNA Sequencing of CAFs

Human patient-derived immortalized CAFs (lines #7 and #37) modified to express either a non-targeting guide RNA (Control) or gRNA targeting ANO6 promoter (ANO6-KD) and dCas9-KRAB constructs (CRISPRi) grown at 100% confluency in 6-well plates were used. Total RNA was isolated using RNeasy Mini Kit (#74104, Qiagen). mRNA libraries were formed by poly-A capture at Novogene and sequenced in a 150 bp paired-end mode using Illumina NovaSeq platform. Reads were aligned to human genome (hg38 version) using TopHat2 and absolute gene counts were quantified using HTSeq. The resulting gene counts were used as input for differential expression analysis between the control and ANO6-KD CAF cells using DESeq2. Genes that are differentially expressed were selected for subsequent downstream analysis for identification of biological pathways and ontologies using Gene Set Enrichment Analysis (GSEA) (p-value<0.001) using MsigDB datasets. To graphically represent the significantly enriched datasets (False Discovery Rate<25%) Enrichment scores and plots were used.

### xxvii. Molecular Modeling of ANO6 and Its Inhibitors

The coordinates of niclosamide bound to TMEM16F (PDBID: 8SUR) was used as a template for docking 3D conformational isomers (“conformers”) of clofazimine in OpenEye Software suite (Cadence Molecular Sciences, Santa Fe, NM, http://www.eyesopen.com). Clofazimine and niclosamide in the context of ANO6 3D folds were overlayed and evaluated for chemical complementarity via rapid overlay of chemical structure. The most complementary clofazimine structure was then energetically minimized using Rosetta as a protein-bound complex. Clofazimine contacts with ANO6 residues were analyzed with ChimeraX software, version 1.8 (://www.rbvi.ucsf.edu/chimerax).

### xxviii. Quantification and Statistical Analysis

For analysis of continuous data, we used Wilcoxon tests, Mann-Whitney and Student t-test as indicated, and binary outcomes were compared using Fisher's exact test. Repeated measures (i.e. multiple measures within a single mouse) were analyzed using generalized linear regression models with Generalized Estimating Equations (GEE). Growth curves were modeled using linear regression with interactions between treatment and time, again using GEE to account for within-sample correlation. Survival time outcomes were assessed using Kaplan-Meier curves with log-rank tests. The statistical details of experiments can be found in the figure legends, figures and text of the Results.

Those skilled in the art will recognize, or be able to ascertain using no more than routine experimentation, many equivalents to the specific embodiments of the method and compositions described herein. Such equivalents are intended to be encompassed by the following claims.

1. A method of treating cancer, comprising administering to a subject in need thereof a calcium channel inhibitor.

2. The method of claim 1, wherein the calcium channel inhibitor targets cancer-associated fibroblasts (CAFs).

3. The method of claim 1, wherein the calcium channel inhibitor does not target a cancer cell.

4. The method of claim 1, wherein the calcium channel inhibitor is an Orai channel inhibitor.

5. The method of claim 4, wherein the Orai channel inhibitor is CM4620, BTP2, Synta-66, RO2959, GSK-7975A, JPIII, N-MeDCPA or any combination thereof.

6. The method of claim 1, wherein Ca+levels in the subject are decreased.

7. The method of claim 1, wherein phospholipid scramblase anoctamin 6 (Ano6) activity in the subject is decreased.

8. The method of claim 1, wherein phosphatidylserine (PtdSer) externalization in the subject is decreased.

9. The method of claim 1, wherein trogocytosis between CAFs and cancer cells in the subject is decreased.

10. The method of claim 1, wherein the immunosuppressive effect of cancer-associated fibroblasts against cytotoxic T cells is attenuated in the subject.

11. The method of claim 1, wherein delivery of exogenous cholesterol to cancer cells is disrupted in the subject.

12. The method of claim 1, further comprising administering an Ano6 inhibitor.

13. The method of claim 12, wherein the Ano6 inhibitor is niclosamide, clofazimine, nitazoxanide, hexachlorophene, 10bm, Monna, Ani9, Ani9 derivative 5f, tannic acid, T16A-A01, dichlorophen, idebenone, shikonin, benzbromarone, CaCC-A01, 9-phenanthrol, niflumic acid, flufenamic acid, talniflumate, A9C, dehydroandrographolide, DIDS, NPPB, or matrine, or any combination thereof.

14. The method of claim 1, further comprising administering an anti-cancer treatment.

15. The method of claim 14, wherein the anti-cancer treatment is radiation, an anti-cancer agent, or a combination thereof.

16. (canceled)

17. The method of claim 1, wherein the cancer is lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma.

18. (canceled)

19. The method of claim 1, wherein the subject has been diagnosed with cancer.

20. The method of claim 19, wherein the cancer is lung cancer, breast cancer, prostate cancer, ovarian cancer, testicular cancer, colon cancer, renal cancer, bladder cancer, pancreatic cancer, glioblastoma, neuroblastoma, retinoblastoma, neuroblastoma, leukemia, melanoma, kidney or renal cancer, or osteosarcoma.

21. A method of decreasing Ano6 activity in a cancer-associated fibroblast (CAF) comprising contacting a CAF with a calcium channel inhibitor, wherein the calcium channel inhibitor reduces Ca+ which is necessary for Ano6 activity, thereby decreasing Ano6 activity in the CAF.

22-27. (canceled)

28. A pharmaceutical composition comprising: an Orai channel inhibitor; an anti-cancer agent and/or an Ano6 inhibitor; and a pharmaceutically acceptable carrier.

29-37. (canceled)
